(19) United States
(12) Patent Application Publication
(10) Pub. No.: US 2022/0288207 A1
(43) Pub. Date: Sep. 15, 2022

(54) PHOTO INDUCED CONTROL OF PROTEIN DESTRUCTION

(71) Applicants: Beth Israel Deaconess Medical Center, Inc., Boston, MA (US); Icahn School of Medicine at Mount Sinai, New York, NY (US)

(72) Inventors: Wenyi WEI, Boston, MA (US); Jian JIN, New York, NY (US); Husnu Umit KANISKAN, New York, NY (US); He CHEN, New York, NY (US); Jing LIU, Boston, MA (US)

(73) Assignees: Beth Israel Deaconess Medical Center, Inc., Boston, MA (US); Icahn School of Medicine at Mount Sinai, New York, NY (US)

(21) Appl. No.: 17/629,707

(22) PCT Filed: Jul. 24, 2020
(86) PCT No.: PCT/US20/43404

§ 371 Date: Jan. 24, 2022

Related U.S. Application Data
(60) Provisional application No. 62/878,583, filed on Jul. 25, 2019.

(51) Int. Cl. A61K 41/00; A61K 47/55; A61K 47/54; A61P 35/00

(52) U.S. Cl. CPC A61K 41/0042; A61K 47/55; A61K 47/545; A61P 35/00

(57) ABSTRACT

By hijacking endogenous E3 ligase to degrade protein targets via the ubiquitin-proteasome system, PROTACs (PRoteolysis TArgeting Chimeras) provide a new strategy to inhibit protein targets that were previously regarded as undruggable. The compounds described herein comprise a photolabile group on PROTACs, enabling the degradation of protein targets in a spatiotemporally controlled manner By adding a photolabile caging group on ubiquitin recruiting moieties, light-inducible protein degradation was acheived. These opto-PROTACs display no activity in the dark, while restricted degradation can be induced at a specific time and rate by UVA-irradiation. Accordingly, these compounds provide light-controlled PROTACs and methods of using such compounds.

### CROSS REFERENCE TO RELATED APPLICATIONS

The present application claims priority to U.S. App. No. 62/878,583, filed Jul. 25, 2019, the entire contents of which are hereby incorporated by reference in their entirety.

### BACKGROUND OF INVENTION

PROteolysis TArgeting Chimera (PROTAC) technique emerged as the result of identifying peptides or small molecule chemical ligands that specifically bind with endogenous E3 ligases, such as β-TRCP, von the Hippel—Lindau tumor suppressor (VHL), Mdm2 and Cereblon (CRBN). Structurally, PROTAC is a bifunctional small molecule that consists of two functional parts, a “warhead” that displays high specificity in binding a protein of interest (POI), and a ligand that is recognized by E3 ligase, where the warhead and the E3 ligase ligand are connected by a linker. PROTACs dictate the POI for proteolysis allowing for the compound to target non-catalytic enzymes with concomitant reduction in drug exposure time and dosage required to suppress signaling. Additionally, PROTACs may eliminate both the catalytic activity and the scaffolding function of bifunctional proteins, such as Receptor Tyrosine Kinases (RTKs).

Pomalidomide and its derivatives, such as lenalidomide and thalidomide, are widely used as immunomodulatory drugs (IMiDs) for treating diseases like multiple myeloma (MM) by inducing the proteolysis of Ikaros family zink finger protein family (IKZF) 1/3 transcriptional factors by the Cereblon (CRBN) E3 ligase. Additionally, these compounds are often used in the ubiquitin recruiting moiety of PROTACs. Essentially, these compounds bind both CRBN and IKZF1/3 to subsequently transfer the ubiquitin chain onto the target proteins.

However, when systemically administered, PROTACs could result in the uncontrolled degradation of POIs in any cells it can access. For example, inhibition of BET bromodomains is relatively well-tolerated while complete loss of BRD2 and BRD4 is lethal. Accordingly, methods for regulating the spatial and/or temporal activation of PROTACs in select tissues and cells are urgently required.

### SUMMARY

In accordance with the foregoing objectives and others, the present disclosure provides compounds capable of recruiting ubiquitin to cells in order to cause photoinduced proteolysis at sites irradiated with certain electromagnetic radiation. The photoinduced degradation of these compounds allows the compounds to recruit ubiquitin and cause degradation of a protein of interest. For example, by installing a light-controllable caging group on the glutarimide NH of pomalidomide to block its recruitment to the CRBN E3 ligase, a general platform to control protein degradation in cells in a highly specific temporal and spatial manner can be achieved using the compounds disclosed herein. In the absence of irradiation, the compounds are inert (or inert or with significantly decreased CRBN E3 ligase recruitment activity). Compounds of the invention comprise inert photolabile groups, which blocking the proteolytic activity until light induced separation of the photolabile groups permits proteolysis of a protein of interest. Typically, this irradiation occurs after binding of the PROTAC comprising a photolabile group to a POI. The photolabile group provides for the temporal and spatial control of proteolysis. Moreover, given the dominating presence of pomalidomide in the synthesis of various PROTACs as an E3 ligase ligand, the caging-uncaging process of opto-pomalidomide may be applied to any other ubiquitin recruitment, and specifically those relying on glutarimide hydrogen bonding PROTACs. Accordingly, the compounds described herein are used for the controllable degradation of protein targets, such as cyclin dependent kinases (CDKs), certain fusion proteins, such as breakpoint cluster region Abelson murine leukemia fusion protein (BCR-ABL), Bruton's tyrosine kinase (BTK), and tau-protein kinases (Tau).

Typically, the compounds disclosed herein have the structure of formula (I):

PB-L-ULB-PLG (I)
wherein ULB is a ubiquitin ligase binding moiety;
L is a linker;
PB is a protein binding moiety; and
PLG is a nitrophenyl based photolabile group (e.g., nitrobenzyl, orthro-nitrobenzyl, nitroveratryloxycarbonyl such as 6-nitroveratryloxycarbonyl, etc.), or pharmaceutically acceptable salts thereof. In certain embodiments, the PLG is covalently bonded to ULB through a carbamate linkage. The PLG may the structure of formula (II):

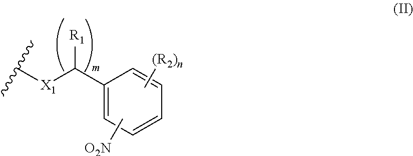

wherein

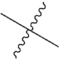

indicates the point of attachment to the ULB group;
m is 0 (i.e., a bond), 1, or 2;
n is 0 (i.e., each R2 is hydrogen), 1, 2, 3, or 4;
X₁ is —O—, —C(O)—, —OC(O)—, —C(O)O—, —NR^{a}C(O)—, or —C(O)NR^{a}—;
R₁ is independently selected at each occurrence from hydrogen, alkyl (e.g., C₁-C₇ alkyl, C₁-C₃ alkyl, etc.), alkoxy (e.g., C₁-C₇ alkoxy, C₁-C₃ alkoxy, etc.);
R₂ is independently selected at each occurrence from hydrogen, —OC(O)R^{e}, —C(O)OR^{e}, —(C(R^{a})(R^{a}))₀₋₄—OC(O)N(R^{a})₂, halogen (e.g., F, Cl, Br, etc.), alkyl (e.g., C₁-C₇ alkyl, C₁-C₃ alkyl, etc.), and alkoxy (e.g., C₁-C₇ alkoxy, C₁-C₃ alkoxy, etc;
R^{a} is independently selected at each occurrence from hydrogen, or alkyl (e.g., C₁-C₇ alkyl, C₁-C₃ alkyl, etc.); and
R^{e} is independently selected at each occurrence from hydrogen, or alkyl (e.g., C₁-C₇ alkyl, C₁-C₃ alkyl, etc.). It will be understood that when n is less than 4, the compound has hydrogen at the remaining positions to satisfy carbon valency of the phenyl group in formula (II). In some embodiments, two vicinal R₂ groups do not together form a ring. In certain implementations, R₁ is not methyl in each occurrence. In some embodiments, the PLG does not have the structure:

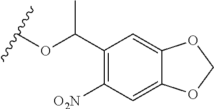 [SMILES: *OC(C)c1cc2c(cc1[N+](=O)[O-])OCO2]

For example, the opto-PROTAC may have the structure of formula (III):

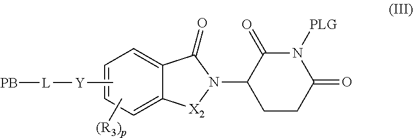

wherein p is 0 (i.e., each R3 is hydrogen), 1, 2, or, 3;
R₃ is independently selected at each occurrence from hydrogen, —N(R^{a})(R^{a}), alkyl (e.g., C₁-C₇ alkyl, C₁-C₃ alkyl, etc.), or alkoxy (e.g., C₁-C₇ alkoxy, C₁-C₃ alkoxy, etc.);
X₂ is C(O), CH, CR^{a}, or NR^{a};
Y is absent (i.e, a bond), —O—, —C(O)—, —OC(O)—, —C(O)O—, —NR^{a}C(O)—, or —C(O)NR^{a}—;
R^{a} is independently selected at each occurrence from hydrogen, or alkyl (e.g., C₁-C₇ alkyl, C₁-C₃ alkyl, etc.). It will be understood that when p is less than 3 in formula (III), the compound has hydrogen bonded at the remaining positions of the phenyl moiety to satisfy carbon valency.

Additionally, non-chimeric compounds are also disclosed. For example, the compound may have the structure formula (VI):

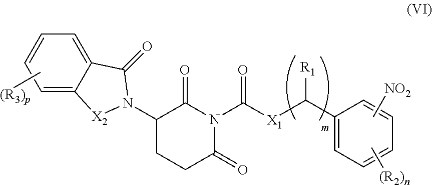

wherein m is 0, 1, or 2;
n is 0, 1, 2, 3, or 4;
p is 0, 1, 2, 3, or 4;
R₁ is independently selected at each occurrence from hydrogen, alkyl (e.g., C₁-C₇ alkyl, C₁-C₃ alkyl, etc.), alkoxy (e.g., C₁-C₇ alkoxy, C₁-C₃ alkoxy, etc.);
R₂ is independently selected at each occurrence from hydrogen, —OC(O)R^{e}, —C(O)OR^{e}, alkyl (e.g., C₁-C₇ alkyl, C₁-C₃ alkyl, etc.), and alkoxy (e.g., C₁-C₇ alkoxy, C₁-C₃ alkoxy, etc.);
R₃ is independently selected at each occurrence from hydrogen, —N(R^{a})(R^{a}), alkyl (e.g., C₁-C₇ alkyl, C₁-C₃ alkyl, etc.), or alkoxy (e.g., C₁-C₇ alkoxy, C₁-C₃ alkoxy, etc.);
X₁ is —O—, —C(O)—, —NR^{a}—, —OC(O)—, —C(O)—, —NR^{a}C(O)—, or —C(O)NR^{a}—;
X₂ is C(O), CH₂, C(R^{a})(R^{a}), or NR^{a};
R^{a} is independently selected at each occurrence from hydrogen, or alkyl (e.g., C₁-C₇ alkyl, C₁-C₃ alkyl, etc.); and
R^{e} is independently selected at each occurrence from hydrogen, or alkyl (e.g., C₁-C₇ alkyl, C₁-C₃ alkyl, etc.);
or pharmaceutically acceptable salts thereof. It will be understood that when n and/or p is less than 4 in formula (VI), the compound has hydrogen bonded at the remaining positions of the relevant moiety to satisfy carbon valency.

Pharmaceutical composition comprising these compounds are also within the present disclosure. For example, the pharmaceutical composition may comprise a compound disclosed herein (e.g., a compound having the structure of formula (I), a compound having the structure of formula (III), a compound having the structure of formula (VI), etc.) and one or more pharmaceutically acceptable carrier, diluents, and/or excipients.

Additionally, methods for the treatment of a proliferative disease are disclosed herein. The method for the treatment of a proliferative disease may comprise the administration of a compound disclosed herein (e.g., a compound having the structure of formula (I), a compound having the structure of formula (III), a compound having the structure of formula (VI), etc.) to a subject in need thereof. Typically, the method may further comprise irradiating the patient with electromagnetic radiation sufficient to induce the separation of the photolabile group from the ubiquitin ligase binding moiety of the compound. In some embodiments, the irradiation comprises photons of one or more wavelengths, a power density, and an irradiation time period sufficient to induce the separation of the photolabile group from the ubiquitin ligase binding moiety of the compound. In some embodiments, the irradiation is able to induce separation of more than 10% or more than 20% or more than 30% or more than 40% or more than 50% or more than 60% or more than 70% or more than 80% or more than 90% or more than 95% or more than 99% or 100% of the photolabile groups in the area irradiated (mol/mol).

### BRIEF DESCRIPTION OF FIGURES

FIG. 1 (panel A) is a depiction of the hydrogen bonding between pomalidomide and CRBN. The key hydrogen bond (black dashes) is formed between glutarimide NH of pomalidomide and backbone carbonyl of His380 of CRBN, based on the structure of DDB1-CRBN E3 ubiquitin ligase in complex with pomalidomide (PDB:4CI3). FIG. 1 (panel B) illustrates the uncaging mechanism of opto-pomalidomide with UVA irradiation at 365 nm. FIG. 1 (panel C) shows the Ultra performance liquid chromatography—tandem mass spectrometer (UPLC-MS) analysis of opto-pomalidomide after irradiation with UVA (365 nm) for 30 minutes in vitro. FIG. 1 (panel D) demonstrates the time-course uncaging of opto-pomalidomide as measured by UVA irradiation in vitro. In several of these figures, Opto-pomalidomide (1 mM) was irradiated with UVA for the indicated time and then subjected to the UV-VIS absorption analysis to determine the photolysis rate. UVA radiation was produced from a UVP UVL-56 handheld UV Lamp (available from fisherscientific) and which was measured toproduced 50 W/m². FIG. 1 (panel E) is a competitive binding pull down assay for opto-pomalidomide. As can be seen, opto-pomalidomide regains the ability to bind with CRBN after UVA irradiation (365 nm, 30 min).

FIG. 2 (panel A) illustrates the synthesis of opto-pomalidomide from pomalidomide. FIG. 2 (panel B). is a schematic illustration for the working model of constitutively active degradation of IKZFs by pomalidomide vs. light inducible degradation of IKZFs by opto-pomalidomide.

FIG. 3 (panel A) is a UPLC chromatogram of pomalidomide. FIG. 3 (panel B). is a UPLC chromatorgram of opto-pomalidomide.

FIG. 4 (panel A) is a mass spectrum of pomalidomide determined from UPLC-MS. FIG. 4 (panel B) is a mass spectrum of opto-pomalidomide determined by UPLC-MS.

FIG. 5 (panel A) is the UV-VIS absorption spectrum of pomalidomide illustrating a maximum in absorption at 390 nm. FIG. 5 (panel B) is the UV-VIS absorption spectrum of opto-pomalidomide illustrating a maximum in absorption at 364 nm. FIG. 5 (panel C) is the UV-VIS absorption of several pomalidomide and opto-pomalidomide mixtures mixed in the different ratios (w/w) illustrating the shift in absorption maximum. FIG. 5 (panel D) is the standard curve of pomalidomide and opto-pomalidomide mixtures comparing the absorbance value at the maximum of the absorbance spectrum for the mixtures. FIG. 5 (panel E) is the UV-VIS absorption of opto-pomalidomide after irradiation with UVA (365 nm) for the indicated time.

FIG. 6 (panel A) is a schematic diagram showing that UVA irradiation activates opto-pomalidomide in cell culture. FIG. 6 (panel B) illustrates that UVA irradiation activates opto-pomalidomide to mediate the interaction between CRBN and IKZF1 as shown by immunoblotting (IB) analysis of whole cell lysis (WCL) and Flag-IP derived from HEK293T cells transfected with the indicated plasmids in the presence of pomalidomide or opto-pomalidomide with/without UVA irradiation (365 nm) for 15 min. Cells were treated with 10 μM of the proteasome inhibitor MG132 for 12 hours before harvest. FIG. 6 (panel C) illustrates that UVA irradiation activates opto-pomalidomide to mediate the ubiquitination of IKZF1 by CRBN in cells as shown by IB analysis of WCL and Ni-NTA pull down products derived from HEK293T cells transfected with the indicated plasmids in the presence of pomalidomide or opto-pomalidomide with/without UVA irradiation (365 nm) for 15 min. Cells were treated with 10 μM MG132 for 12 hours before harvest. FIG. 6 (panel D) illustrates that opto-pomalidomide does not promote the degradation of IKZF1/3 without UVA irradiation as shown by IB analysis of WCL derived from MNI1S-CRBN^{+/+} vs. MM1S-CRBN^{-/-} cells in the presence of pomalidomide or opto-pomalidomide for 12 hours.

FIG. 6 (panel E) demonstrates that UVA irradiation activates opto-pomalidomide to promote the degradation of IKZF1/3 in cells as shown by IB analysis of WCL derived from MM1S cells in the presence of opto-pomalidomide with UVA irradiation (365 nm) for the indicated time. FIG. 6 (panel F) shows that UVA irradiation-activated opto-pomalidomide inhibits MM1S cell proliferation in a dose-dependent manner. MM1S cells were treated by pomalidomide vs. opto-pomalidomide with or without UVA irradiation (365 nm) for 15 min, and then subjected to CCK-8 cell viability assay. FIG. 6 (panel G) shows pomalidomide reduces MM1S cell proliferation in a CRBN-dependent manner. MM1S-CRBN^{+/+} and MM1S-CRBN^{-/-} cells were treated by pomalidomide vs. opto-pomalidomide for 72 hours, and then subjected to CCK-8 cell viability assay.

FIG. 7 (panel A) shows that UVA irradiation activates opto-pomalidomide to mediate IKZF1 degradation in MT2 cells through IB experimentation. FIG. 7 (panel B) shows that UVA irradiation (365nm) alone does not lead to degradation of IKZF1/3 in MM1.S through IB experimentation. FIG. 7 (panel C) shows that UVA irradiation (365nm) alone does not lead to degradation of IKZF1/3 in 293FT cells through IB experimentation. FIG. 7 (panel D) compares the cell viability as a function of compound concentration and illustrates that UVA irradiation-activated opto-pomalidomide inhibits MT2 cell proliferation in a dose-dependent manner. MT2 cells were treated by pomalidomide vs. opto-pomalidomide with or without UVA irradiation (365 nm) for 15 min, and then subjected to CCK-8 cell viability assay.

FIG. 8 (panel A) illustrates the synthesis of opto-dBET1. FIG. 8 (panel B) is a schematic illustration for the working model of dBET1 vs. opto-dBET1 vs. opto-dBET1+UVA irradiation on promoting BRDs degradation.

FIG. 9 (panel A) is a schematic illustration of the chemical structure of opto-dBET1. FIG. 9 (panel B) illustrates the time-course uncaging of opto-dBET1 by UVA irradiation in vitro. Opto-dBET1 (1 mM) was irradiated with UVA (365 nm) for indicated time and then subjected to the UV-VIS absorption analysis. Cells were treated with 10 μM MG132 for 12 hours before harvest. FIG. 9 (panels C-D) demonstrate that irradiation activates opto-dBET1 to mediate the ubiquitination of BRD2 (C) and BRD3 (D) by CRBN in cells as demonstrated by D3 analysis of WCL and Ni-NTA pull down products derived from HEK293T cells transfected with indicated plasmids in the presence of dBET1 or opto-dBET1 with/without UVA irradiation (365 nm) for 15 min. Cells were treated with 10 μM MG132 for 12 hours before harvest. FIG. 9 (panel E) illustrates that dBET1 promotes the degradation of BRDs in a CRBN dependent manner as shown by IB analysis of WCL derived from 293FT-CRBN^{+/+} vs. 293FT-CRBN^{-/-} treated with dBET1 at indicated dose for 12 hours. FIG. 9 (panel F) demonstrates that opto-dBET1 does not promote the degradation of BRDs in cells without UVA irradiation as shown by IB analysis of WCL derived from 293FT-CRBN^{+/+} vs. 293FT-CRBN^{+/+} treated with opto-dBET1 at indicated dose for 12 hours. FIG. 9 (panels G-H) illustrate that UVA irradiation activates opto-dBET1 to promote the degradation of BRDs in cells in a CRBN-dependent manner as shown by IB analysis of WCL derived from 293FT-CRBN^{+/+} (G) vs. 293FT-CRBN^{-/-} (H) in the presence of dBET1 vs. opto-dBET1 with UVA irradiation (365 nm) for 5 or 15 minutes. FIG. 9 (panel I) illustrates that UVA irradiation-activated opto-dBET1 promotes BRD3 degradation in a UPS-dependent manner as shown by IB analysis of WCL derived from 293FT-CRBN^{+/+} vs. 293FT-CRBN^{-/-} in the presence of dBET1 vs. opto-dBET1 with or without UVA irradiation (365 nm). Cell were treated with either 10 μM MG132 or 1 μM MLN4924 for 12 hours. FIG. 9 (panel J-K) demonstrate that UVA irradiation-activated opto-dBET1 inhibits HEK293FT (J) and C4-2 (K) cell proliferation in a dose-dependent manner. HEK293FT cells were treated by dBET1 vs. opto-dBET1 with or without UVA irradiation (365 nm) for 15 minutes, and then subjected to CCK-8 cell viability assay.

FIG. 10 (panel A) is UPLC chromatogram and analysis of dBET1. FIG. 10 (panel B) illustrates UPLC analysis of opto-dBET1. FIG. 10 (panel C) illustrates the UPLC analysis of opto-dBET1 after irradiation with UVA (365nm) for 30 minutes.

FIG. 11 (panel A) is a mass spectrum of dBET1 determined by UPLC-MS. FIG. 11 (panel B) is mass spectrum of opto-dBET1 determined by UPLC-MS.

FIG. 12 (panel A) is the UV-VIS absorption spectrum of dBET1. FIG. 12 (panel B) is the UV-VIS absorption spectrum of opto-dBET1. FIG. 12 (panel C) is the UV-VIS absorption spectrum of dBET1 and opto-dBET1 mixtures. FIG. 12 (panel D) is the standard correlation curve of dBET1 and opto-dBET1. FIG. 12 (panel E) illustrates the UV-VIS absorption of opto-dBET1 after irradiation with UVA (365nm) for indicated time.

FIG. 13 (panel A) shows that dBET1 inhibits cell proliferation in 293FT-CRBN^{+/+} but not 293FT-CRBN^{-/-} cells. FIG. 13 (panel B) shows that opto-dBET1 did not inhibit cell proliferation in 293FT-CRBN^{+/+} and 293FT-CRBN^{-/-} cells. 293FT-CRBN^{+/+} and 293FT-CRBN^{-/-} cells were treated by dBET1 vs. opto-dBET1 for 72 hours, and then subjected to CCK-8 cell viability assay.

FIG. 14 (panel A) illustrates the synthesis of opto-dALK. FIG. 14 (panel B) is a schematic illustration for the working model of dALK vs. opto-dALK vs. opto-dALK+UVA irradiation (365nm) on promoting the degradation of ALK fusion proteins.

FIG. 15 (panel A) is a schematic illustration of the chemical structure of the engineered opto-dALK. FIG. 15 (panel B) shows the time-course uncaging of opto-dALK by UVA irradiation in vitro. Opto-dBET1 (1 mM) was irradiated with UVA (365 nm) for indicated time and then subjected to the UV-VIS absorption analysis. FIG. 15 (panels C-D) illustrate that UVA irradiation activates opto-dALK to promote the degradation of EML-ALK fusion proteins in cells as demonstrated by D3 analysis of WCL derived from NCI-H2228 (C) or NCI-3122 (D) NSCLC cells treated with BET1 vs. opto-dBET1 at indicated dose with or without UVA irradiation (365 nm) for 5 or 15 minutes. FIG. 15 (panels E-F) illustrate that UVA irradiation-activated opto-dALK inhibits H2228 (E) or NCI-3122 (F) cell proliferation in a dose-dependent manner. NCI-H2228 cells were treated by dALK vs. opto-dALK with or without UVA irradiation (365 nm) for 15 minutes, and then subjected to CCK-8 cell viability assay.

FIG. 16 (panel A) shows the UPLC chromatogram and analysis of dALK. FIG. 16 (panel B) shows the UPLC chromatogram and analysis of opto-dALK. FIG. 16 (panel C) shows the UPLC chromatogram and analysis of opto-dALK after irradiation with UVA (365 nm) for 30 minutes.

FIG. 17 (panel A) shows the mass spectrum of dALK as determined by UPLC-MS. FIG. 17 (panel B) shows the mass spectrum of opto-dALK as determined by by UPLC-MS.

FIG. 18 (panel A) shows the UV-VIS absorption spectrum of dALK. FIG. 18 (panel B) shows the UV-VIS absorption spectrum of opto-dALK. FIG. 18 (panel C) shows the UV-VIS absorption spectrum of dALK and opto-dALK mixtures. FIG. 18 (panel D) shows the standard curve of dALK and opto-dALK. FIG. 18 (panel E) is the UV-VIS absorption of opto-dALK after irradiation with UVA (365nm) for indicated time.

FIG. 19 (panels A-B) show that dALK promotes the degradation of EML4-ALK fusion proteins in NCI-H2228 (A) and NCI-H3122 (B) NSCLC cell lines. FIG. 19 (panels C-D) show that opto-dALK is inefficient in inhibiting the proliferation of H2228 (C) or NCI-3122 (D). NCI-H2228 and NCI-H3122. NSCLC cell lines were treated by dALK vs. opto-dALK for 72 hours, and then subjected to CCK-8 cell viability assay.

FIG. 20 is a general mechanism for opto-PROTAC and opto-pomalidomide functioning wherein irradiation with light at certain frequencies results in decaging of the ubqiquitin function moiety of the compounds allowing for subsequent ubiquitin recruitment and cell degradation.

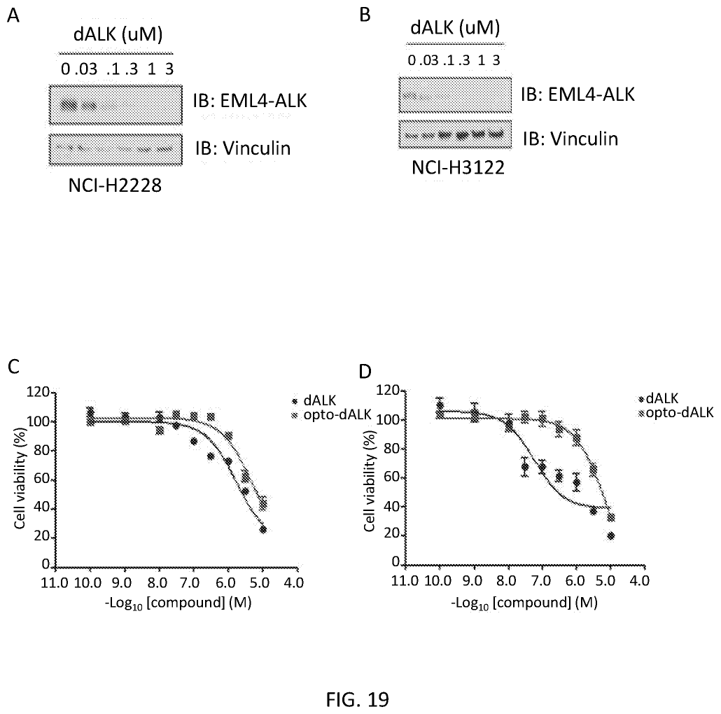

It will be understood that the figure panels may be referenced herein by the figure number and the panel number. For example, FIG. 19 (panel A) may be referenced herein as FIG. 19A.

### DETAILED DESCRIPTION

Detailed embodiments of the present invention are disclosed herein; however, it is to be understood that the disclosed embodiments are merely illustrative of the invention that may be embodied in various forms. In addition, each of the examples given in connection with the various embodiments of the invention is intended to be illustrative, and not restrictive.

All terms used herein are intended to have their ordinary meaning in the art unless otherwise provided. All concentrations are in terms of percentage by weight of the specified component relative to the entire weight of the topical composition, unless otherwise defined.

As used herein, “a” or “an” shall mean one or more. As used herein when used in conjunction with the word “comprising,” the words “a” or “an” mean one or more than one. As used herein “another” means at least a second or more.

As used herein, all ranges of numeric values include the endpoints and all possible values disclosed between the disclosed values. Moreover, all values that fall within these ranges, as well as the upper or lower limits of a range of values, are also contemplated by the present application. For example, the exact values of all half-integral numeric values are also contemplated as specifically disclosed and as limits for all subsets of the disclosed range. For example, a range of from 0.1% to 3% specifically discloses a percentage of 0.1%, 1%, 1.5%, 2.0%, 2.5%, and 3%. Additionally, a range of 0.1 to 3% includes subsets of the original range including from 0.5% to 2.5%, from 1% to 3%, from 0.1% to 2.5%, etc. It will be understood that sum of all weight percentages does not exceed 100% unless otherwise specified.

By “consist essentially” it is meant that the ingredients include only the listed components along with the normal impurities present in commercial materials and with any other additives present at levels which do not affect the operation of the invention, for instance at levels less than 5% by weight or less than 1% or even 0.5% by weight.

As termed herein as “opto-” compounds such as opto-pomalidomide, opto-PROTACs, etc. are compounds comprising a photolabile group.

Typically, alkyl groups described herein refer to a branched or straight-chain monovalent saturated aliphatic hydrocarbon radical of 1-30 carbon atoms (e.g., 1-16 carbon atoms, 6-20 carbon atoms, 8-16 carbon atoms, or 4-18 carbon atoms, 4-12 carbon atoms, etc.). In some embodiments, the alkyl group may be substituted with 1, 2, 3, or 4 substituent groups as defined herein. Alkyl groups may have from 1-26 carbon atoms. In other embodiments, alkyl groups will have from 6-18 or from 1-8 or from 1-6 or from 1-4 or from 1-3 carbon atoms, including for example, embodiments having one, two, three, four, five, six, seven, eight, nine, or ten carbon atoms. Any alkyl group may be substituted or unsubstituted. Examples of alkyl groups include methyl, ethyl, propyl, butyl, pentyl, hexyl, heptyl, octyl, nonyl, decyl, undecyl, and dodecyl groups. Heteroalkyl groups may refer to branched or straight-chain monovalent saturated aliphatic hydrocarbon radicals with one or more heteroatoms (e.g., N, O, S, etc.) in the carbon chain. Heteroalkyl groups may have 1-30 carbon atoms (e.g., 1-16 carbon atoms, 6-20 carbon atoms, 8-16 carbon atoms, or 4-18 carbon atoms, 4-12 carbon atoms, etc.). In some embodiments, the heteroalkyl group may be substituted with 1, 2, 3, or 4 substituent groups as defined herein. Heteroalkyl groups may have from 1-26 carbon atoms. In other embodiments, heteroalkyl groups will have from 6-18 or from 1-8 or from 1-6 or from 1-4 or from 1-3 carbon atoms, including for example, embodiments having one, two, three, four, five, six, seven, eight, nine, or ten carbon atoms. In some embodiments, the heteroalkyl group can be further substituted with 1, 2, 3, or 4 substituent groups as described herein for alkyl groups. Examples of heteroalkyl groups are an “alkoxy” which, as used herein, refers alkyl-O—; and “alkoyl” which, as used herein, refers to alkyl-CO—. Alkoxy substituent groups or alkoxy-containing substituent groups may be substituted by, for example, one or more alkyl groups.

Aryl groups may be aromatic mono- or polycyclic radicals of 6 to 12 carbon atoms having at least one aromatic ring. Examples of such groups include, but are not limited to, phenyl, naphthyl, 1,2,3,4-tetrahydronaphthalyl, 1,2-dihydronaphthalyl, indanyl, and 1H-indenyl. Typically, heteroaryls include mono- or polycyclic radical of 5 to 12 atoms having at least one aromatic ring containing one, two, or three ring heteroatoms selected from N, O, and S, with the remaining ring atoms being C. One or two ring carbon atoms of the heteroaryl group may be replaced with a carbonyl group. Examples of heteroaryl groups are pyridyl, benzooxazolyl, benzoimidazolyl, and benzothiazolyl.

Similarly, alkylene groups refer to a straight or branched chain divalent hydrocarbon radical having from one to ten carbon atoms, optionally substituted with substituents selected from the group which includes lower alkyl (e.g., C₁-C₄, etc.), lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen and lower perfluoroalkyl, multiple degrees of substitution being allowed. Examples of alkylene as used herein include, but are not limited to, methylene, ethylene, n-propylene, n-butylene, and the like. Alkylene groups may be saturated or unsaturated. Heteroalkylene groups may be alkylene groups comprising one or more heteroatoms (e.g., N, S, O, etc.) in the carbon chain. Cycloalkylene groups may be divalent hydrocarbons comprising one or more saturated or unsaturated cycloalkyl groups. The two points of attachment on cycloalkylene groups may be at two points in the ring, for example, at vicinal positions, germinal positions. Cycloalkylene groups may be divalent saturated mono- or multicyclic ring system, in certain embodiments of 3 to 10 carbon atoms, in other embodiments 3 to 6 carbon atoms; cycloalkenylene and cycloalkynylene refer to divalent mono- or multicyclic unsaturated ring systems that respectively include at least one double bond and at least one triple bond. Cycloalkylene, Cycloalkenylene and cycloalkynylene groups may, in certain embodiments, contain 3 to 10 carbon atoms, with cycloalkenylene groups in certain embodiments containing 4 to 7 carbon atoms and cycloalkynylene groups in certain embodiments containing 8 to 10 carbon atoms. The ring systems of the cycloalkylene, cycloalkenylene and cycloalkynylene groups may be composed of one ring or two or more rings which may be joined together in a fused, bridged or spiro-connected fashion. Heterocyclene groups may be divalent monocyclic or multicyclic non-aromatic ring system, in certain embodiments of 3 to 10 members, in one embodiment 4 to 7 members, in another embodiment 5 to 6 members, where one or more, including 1 to 3, of the atoms in the ring system is a heteroatom, that is, an element other than carbon, including but not limited to, nitrogen, oxygen or sulfur.

Arylene groups may be monocyclic or polycyclic, in certain embodiments monocyclic, divalent aromatic group, in one embodiment having from 5 to about 20 carbon atoms and at least one aromatic ring, in another embodiment 5 to 12 carbons. In further embodiments, arylene includes lower arylene. Arylene groups include, but are not limited to, 1,2-, 1,3- and 1,4-phenylene. Heteroarylene groups are typically divalent monocyclic or multicyclic aromatic ring systems, in one embodiment of about 5 to about 15 atoms in the ring(s), where one or more, in certain embodiments 1 to 3, of the atoms in the ring system is a heteroatom, that is, an element other than carbon, including but not limited to, nitrogen, oxygen or sulfur.

The term “substituent” refers to a group “substituted” on, e.g., an alkyl, at any atom of that group, replacing one or more hydrogen atoms therein. In some aspects, the substituent(s) on a group are independently any one single, or any combination of two or more of the permissible atoms or groups of atoms delineated for that substituent. In another aspect, a substituent may itself be substituted with any one of the substituents described herein.

A substituted hydrocarbon group may have as a substituent one or more hydrocarbon radicals, substituted hydrocarbon radicals, or may comprise one or more heteroatoms. Examples of substituted hydrocarbon radicals include, without limitation, heterocycles, such as heteroaryls. Unless otherwise specified, a hydrocarbon substituted with one or more heteroatoms will comprise from 1-20 heteroatoms. In other embodiments, a hydrocarbon substituted with one or more heteroatoms will comprise from 1-12 or from 1-8 or from 1-6 or from 1-4 or from 1-3 or from 1-2 heteroatoms. Examples of heteroatoms include, but are not limited to, oxygen, nitrogen, sulfur, phosphorous, halogen (e.g., F, Cl, Br, I, etc.), boron, silicon, etc. In some embodiments, heteroatoms will be selected from the group consisting of oxygen, nitrogen, sulfur, phosphorous, and halogen (e.g., F, Cl, Br, I, etc.). In some embodiments, a heteroatom or group may substitute a carbon. In some embodiments, a heteroatom or group may substitute a hydrogen. In some embodiments, a substituted hydrocarbon may comprise one or more heteroatoms in the backbone or chain of the molecule (e.g., interposed between two carbon atoms, as in “oxa”). In some embodiments, a substituted hydrocarbon may comprise one or more heteroatoms pendant from the backbone or chain of the molecule (e.g., covalently bound to a carbon atom in the chain or backbone, as in “oxo”).

In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls. Moreover, where a moiety is substituted with an R substituent, the group may be referred to as “R-substituted.” Where a moiety is R-substituted, the moiety is substituted with at least one R substituent and each R substituent is optionally different.

Unless otherwise noted, all groups described herein (e.g., alkyl, cycloalkyl, heteroalkyl, heterocycloalkyl, aryl, heteroaryl, alkylene, heteroalkylene, cylcoalkylene, heterocycloalkylene, etc.) may optionally contain one or more common substituents, to the extent permitted by valency. Common substituents include halogen (e.g., F, Cl, etc.), C₁₋₁₂ straight chain or branched chain alkyl, C₂₋₁₂ alkenyl, C₂₋₁₂ alkynyl, C₃₋₁₂ cycloalkyl, C₆₋₁₂ aryl, C₃₋₁₂ heteroaryl, C₃₋₁₂ heterocyclyl, C₁₋₁₂ alkylsulfonyl, nitro, cyano, —COOR, —C(O)NRR′, —OR, —SR, —NRR′, and oxo, such as mono- or di- or tri-substitutions with moieties such as halogen, fluoroalkyl, perfluoroalkyl, perfluroalkoxy, trifluoromethoxy, chlorine, bromine, fluorine, methyl, methoxy, pyridyl, furyl, triazyl, piperazinyl, pyrazoyl, imidazoyl, and the like, each optionally containing one or more heteroatoms such as halo, N, O, S, and P. R and R′ are independently hydrogen, C₁₋₁₂ alkyl, C₁₋₁₂ haloalkyl, C₂₋₁₂ alkenyl, C₂₋₁₂ alkynyl, C₃₋₁₂ cycloalkyl, C₄₋₂₄ cycloalkylalkyl, C₆₋₁₂ aryl, C₇₋₂₄ aralkyl, C₃₋₁₂ heterocyclyl, C₃₋₂₄ heterocyclylalkyl, C₃₋₁₂ heteroaryl, or C₄₋₂₄ heteroarylalkyl. Further, as used herein, the phrase optionally substituted indicates the designated hydrocarbon group may be unsubstituted (e.g., substituted with H) or substituted. Typically, substituted hydrocarbons are hydrocarbons with a hydrogen atom removed and replaced by a substituent (e.g., a common substituent). It is understood by one of ordinary skill in the chemistry art that substitution at a given atom is limited by valency. The use of a substituent (radical) prefix names such as alkyl without the modifier optionally substituted or substituted is understood to mean that the particular substituent is unsubstituted. However, the use of haloalkyl without the modifier optionally substituted or substituted is still understood to mean an alkyl group, in which at least one hydrogen atom is replaced by halo.

The term “pharmaceutical composition,” as used herein, represents a composition containing a compound described herein formulated with a pharmaceutically acceptable excipient. In some embodiments, the pharmaceutical composition is manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gel cap, or syrup); for topical administration (e.g., as a cream, gel, lotion, or ointment); for intravenous administration (e.g., as a sterile solution free of particulate emboli and in a solvent system suitable for intravenous use); or in any other formulation described herein (see below).

Useful pharmaceutical carriers for the preparation of the compositions hereof, can be solids, liquids, or gases. Thus, the compositions can take the form of tablets, pills, capsules, suppositories, powders, enterically coated or other protected formulations (e.g., binding on ion-exchange resins or packaging in lipid-protein vesicles), sustained release formulations, solutions, suspensions, elixirs, and aerosols. The carrier can be selected from the various oils including those of petroleum, animal, vegetable or synthetic origin, e.g., peanut oil, soybean oil, mineral oil, and sesame oil. Water, saline, aqueous dextrose, and glycols are preferred liquid carriers, particularly (when isotonic with the blood) for injectable solutions. For example, formulations for intravenous administration comprise sterile aqueous solutions of the active ingredient(s) which are prepared by dissolving solid active ingredient(s) in water to produce an aqueous solution and rendering the solution sterile. Suitable pharmaceutical excipients include starch, cellulose, chitosan, talc, glucose, lactose, gelatin, malt, rice, flour, chalk, silica, magnesium stearate, sodium stearate, glycerol monostearate, sodium chloride, dried skim milk, glycerol, propylene glycol, water, and ethanol. The compositions may be subjected to conventional pharmaceutical additives such as preservatives, stabilizing agents, wetting or emulsifying agents, salts for adjusting osmotic pressure, and buffers. Suitable pharmaceutical carriers and their formulation are described in Remington's Pharmaceutical Sciences by E. W. Martin. Such compositions will, in any event, contain an effective amount of the active compound together with a suitable carrier so as to prepare the proper dosage form for administration to the recipient.

As used herein, the term “pharmaceutically acceptable salt” refers to salts of any of the compounds described herein that within the scope of sound medical judgment, are suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. Salts may be prepared from pharmaceutically acceptable non-toxic acids and bases including inorganic and organic acids and bases. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, dichloroacetate, digluconate, dodecyl sulfate, ethanesulfonate, formate, fumarate, glucoheptonate, glutamate, glycerophosphate, hemi sulfate, heptonate, hexanoate, hippurate, hydrobromide, hydrochloride, hydroiodide, 2-hydroxy-ethanesulfonate, isethionate, lactobionate, lactate, laurate, lauryl sulfate, malate, maleate, malonate, mandelate, methanesulfonate, mucate, 2-naphthalenesulfonate, nicotinate, nitrate, oleate, oxalate, palmitate, pamoate, pantothenate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative basic salts include alkali or alkaline earth metal salts include sodium, lithium, potassium, calcium, and magnesium, aluminum salts, as well as nontoxic ammonium, quaternary ammonium, and amine cations, including, but not limited to ammonium, tetramethylammonium, tetraethylammonium, methylamine, dimethylamine, trimethylamine, triethylamine, caffeine, and ethylamine. As used herein, the term “subject” refers to any organism to which a composition in accordance with the disclosure may be administered, e.g., for experimental, diagnostic, prophylactic, and/or therapeutic purposes. In most embodiments, the subject is a human. Other subjects may include mammals such as mice, rats, rabbits, cats, dogs, non-human primates. The subject may be domesticated animals (e.g., cows, calves, sheep, goat, lambs, horses, poultry, foals, pigs, piglets, etc.), or animals in the family Muridae (e.g., rats, mice, etc.), or animals in the family Felidae. A subject may seek or be in need of treatment, require treatment, be receiving treatment, may be receiving treatment in the future, or a human or animal that is under care by a trained professional for a particular disease or condition (e.g., cancer, etc.).

It will be understood that any divalent linking moiety with multiple substituent parts (each typically indicated with “—”) may be attached to the specified moieties in either direction to the extent permitted by valency, unless otherwise indicated. For example, a linking moiety having the structure -L₁-L₂- may be used to link two portions of a compound in the -L₁-L₂- orientation or in the -L₂-L₁- orientation.

Typically, a proliferative disease refers to the physiological condition in a subject characterized by unregulated cell growth such as cancer. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small cell lung cancer, non-small cell lung cancer (“NSCLC”), vulval cancer, thyroid cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, hepatic carcinoma, anal carcinoma, penile carcinoma, as well as head and neck cancer. In yet other embodiments, the cancer is at least one selected from the group consisting of ALL, T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitts Lymphoma, B-cell ALL, Philadelphia chromosome positive ALL, Philadelphia chromosome positive CML, lymphoma, leukemia, multiple myeloma, myeloproliferative diseases, large B cell lymphoma, and B cell Lymphoma.

The term “unit dosage form” refers to a physically discrete unit suitable as a unitary dosage for human subjects and other mammals, each unit containing a predetermined quantity of active material calculated to produce the desired therapeutic effect, in association with any suitable pharmaceutical excipient or excipients. Exemplary, non-limiting unit dosage forms include a tablet (e.g., a chewable tablet), caplet, capsule (e.g., a hard capsule or a soft capsule), lozenge, film, strip, gel cap, and syrup.

Compounds provided herein can have one or more asymmetric carbon atoms and can exist in the form of optically pure enantiomers, mixtures of enantiomers such as racemates, optically pure diastereoisomers, mixtures of diastereoisomers, diastereoisomeric racemates or mixtures of diastereoisomeric racemates. The optically active forms can be obtained for example by resolution of the racemates, by asymmetric synthesis or asymmetric chromatography (chromatography with a chiral adsorbent or eluant). That is, certain of the disclosed compounds may exist in various stereoisomeric forms. Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms and represent the configuration of substituents around one or more chiral carbon atoms. Enantiomers of a compound can be prepared, for example, by separating an enantiomer from a racemate using one or more well-known techniques and methods, such as chiral chromatography and separation methods based thereon. The appropriate technique and/or method for separating an enantiomer of a compound described herein from a racemic mixture can be readily determined by those of skill in the art. “Racemate” or “racemic mixture” means a mixture containing two enantiomers, wherein such mixtures exhibit no optical activity; i.e., they do not rotate the plane of polarized light. “Geometric isomer” means isomers that differ in the orientation of substituent atoms (e.g., to a carbon-carbon double bond, to a cycloalkyl ring, to a bridged bicyclic system, etc.). Atoms (other than H) on each side of a carbon-carbon double bond may be in an E (substituents are on opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in atropisomeric forms. Atropisomers are stereoisomers resulting from hindered rotation about single bonds where the steric strain barrier to rotation is high enough to allow for the isolation of the conformers. The compounds disclosed herein may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods.

When the stereochemistry of a disclosed compound is named or depicted by structure, the named or depicted stereoisomer is at least 60%, 70%, 80%, 90%, 99%, or 99.9%) by weight relative to the other stereoisomers. When a single enantiomer is named or depicted by structure, the depicted or named enantiomer is at least 60%, 70%, 80%, 90%, 99%, or 99.9% by weight optically pure. When a single diastereomer is named or depicted by structure, the depicted or named diastereomer is at least 60%, 70%, 80%, 90%, 99%, or 99.9% by weight pure. Percent optical purity is the ratio of the weight of the enantiomer or over the weight of the enantiomer plus the weight of its optical isomer. Diastereomeric purity by weight is the ratio of the weight of one diastereomer or over the weight of all the diastereomers. When the stereochemistry of a disclosed compound is named or depicted by structure, the named or depicted stereoisomer is at least 60%, 70%, 80%, 90%, 99%, or 99.9% by mole fraction pure relative to the other stereoisomers. When a single enantiomer is named or depicted by structure, the depicted or named enantiomer is at least 60%, 70%, 80%, 90%, 99%, or 99.9% by mole fraction pure. When a single diastereomer is named or depicted by structure, the depicted or named diastereomer is at least 60%, 70%, 80%, 90%, 99%, or 99.9% by mole fraction pure. Percent purity by mole fraction is the ratio of the moles of the enantiomer or over the moles of the enantiomer plus the moles of its optical isomer. Similarly, percent purity by moles fraction is the ratio of the moles of the diastereomer or over the moles of the diastereomer plus the moles of its isomer. When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s) or mixtures of diastereomers in which one or more diastereomer is enriched relative to the other diastereomers. The disclosure embraces all of these forms.

It will be understood that the description of compounds herein is limited by principles of chemical bonding known to those skilled in the art. Accordingly, where a group may be substituted by one or more of a number of substituents, such substitutions are selected so as to comply with principles of chemical bonding with regard to valencies, etc., and to give compounds which are not inherently unstable. For example, any carbon atom will be bonded to two, three, or four other atoms, consistent with the four valence electrons of carbon. Additionally, when a structure has less than the required number of functional groups indicated, those carbon atoms without an indicated functional group are bonded to the requisite number of hydrogen atoms to satisfy the valency of that carbon.

The term “effective amount” or “therapeutically effective amount” of an agent, as used herein, is that amount sufficient to effect beneficial or desired results, such as clinical results, and, as such, an “effective amount” depends upon the context in which it is being applied. In one embodiment, an effective amount is the amount of an irradiated compound described herein sufficient to effect the degradation of a protein of interest. In another embodiment, in the context of administering an agent that is an anticancer agent, an effective amount of an agent is, for example, an amount sufficient to achieve alleviation or amelioration or prevention or prophylaxis of one or more symptoms or conditions; diminishment of extent of disease, disorder, or condition; stabilized (i.e., not worsening) state of disease, disorder, or condition; preventing spread of disease, disorder, or condition; delay or slowing the progress of the disease, disorder, or condition; amelioration or palliation of the disease, disorder, or condition (e.g., cancer, etc.); and remission (whether partial or total), whether detectable or undetectable, as compared to the response obtained without administration of the agent. In some embodiments, the effective amount assumes that more than 50% of the compounds administered release the photolabile group under irradiation conditions (e.g., more than 60%, more than 70%, more than 80%, more than 90%, more than 95%, more than 99%, 100%, etc.) to achieve the active compound capable of degrading a protein of interest.

Typically, the compound may have the structure of formula (I):

PB-L-ULB—PLG (I)
wherein ULB is a ubiquitin ligase binding moiety;
L is a linker;
PB is a protein binding moiety; and
PLG is a nitrophenyl based photolabile group (e.g., nitrobenzyl, orthro-nitrobenzyl, nitroveratryloxycarbonyl such as 6-nitroveratryloxycarbonyl, etc.),
wherein PLG is covalently bonded to ULB through a carbamate linkage;
or pharmaceutically acceptable salts thereof. In various embodiments, the nitrogen of the carbamate linkage is a hydrogen binding moiety in ULB when the photolabile group is not present. In certain implementations the nitrogen of the carbamate linkage is the nitrogen in a glutarimide moiety. The PLG may have the structure of formula (II):

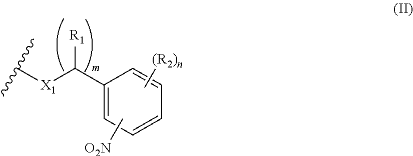

wherein

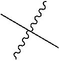

indicates the point of attachment to the ULB group;
m is 0 (i.e., a bond), 1, or 2;
n is 0, 1, 2, 3, or 4;
X₁ is —O—, —C(O)—, —OC(O)—, —C(O)O—, —NR^{a}C(O)—, or —C(O)NR^{a}—;
R₁ is independently selected at each occurrence from hydrogen, alkyl (e.g., C₁-C₇ alkyl, C₁-C₃ alkyl, etc.), alkoxy (e.g., C₁-C₇ alkoxy, C₁-C₃ alkoxy, etc.);
R₂ is independently selected at each occurrence from hydrogen, —OC(O)R^{e}, —C(O)R^{e}, —(C(R^{a})(R^{a}))₀₋₄—OC(O)N(R^{a})₂, halogen (e.g., F, Cl, Br, etc.), alkyl (e.g., C₁-C₇ alkyl, C₁-C₃ alkyl, etc.), and alkoxy (e.g., C₁-C₇ alkoxy, C₁-C₃ alkoxy, etc.), wherein two vicinal R₂ groups do not together form a ring;
R^{a} is independently selected at each occurrence from hydrogen, or alkyl (e.g., C₁-C₇ alkyl, C₁-C₃ alkyl, etc.); and
R^{e} is independently selected at each occurrence from hydrogen, or alkyl (e.g., C₁-C₇ alkyl, C₁-C₃ alkyl, etc.). For example, the PLG group may have the structure of formula (IIa):

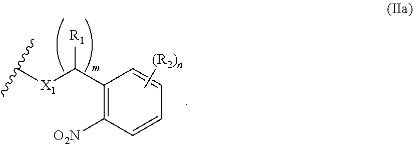

In certain implementations, n is 2 and at least one R₂ is alkoxy (e.g., C₁-C₃ alkoxy such as methoxy, etc.). In various aspects, m is 1 and R₁ is hydrogen.

The compound may comprise a PLG group having the structure of formula (IIb) or (IIc):

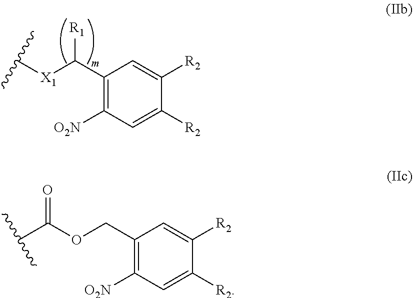

In specific embodiments, each R2 (e.g., in formula (I%) or in formula (IIc)) is methoxy.

Various compounds capable of recruiting ubiquitin may be used in the compounds disclosed herein. Typically, the compound comprises a moiety derived from one of these structures wherein the indicated required groups (e.g., L, PLG, etc.) are linked to the ULB structure through what is a hydrogen in the underivatized structure. The derivitized moiety may also be substituted one or more times in any other hydrogen locations. For example, in some embodiments, the ULB moiety binds to an E3 ubiquitin ligase. The E3 ubiquitin ligase may comprise von Hippel Lindau (VHL) E3 ubiquitin ligase, β-Transducin Repeat Containing (β-TRCP) E3 Ubiquitin Protein Ligase, Mouse Double Minute 2 (Mdm2) E3 Ubiquitin Protein Ligase, or a Cereblon (CRBN) E3 Ubiquitin ligase. In certain implementations, the compound has the structure of formula (III):

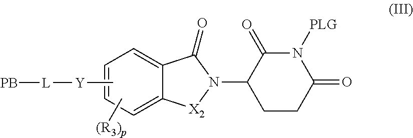

wherein p is 0, 1, 2, or, 3;
R₃ is independently selected at each occurrence from hydrogen, —N(R^{a})(R^{a}), alkyl (e.g., C₁-C₇ alkyl, C₁-C₃ alkyl, etc.), or alkoxy (e.g., C₁-C₇ alkoxy, C₁-C₃ alkoxy, etc.);
X₂ is C(O), CH, CR^{a}, or NR^{a};
Y is absent (i.e, a bond), —O—, —C(O)—, —OC(O)—, —C(O)O—, —NR^{a}C(O)—, or —C(O)NR^{a}—;
R^{a} is independently selected at each occurrence from hydrogen, or alkyl (e.g., C₁-C₇ alkyl, C₁-C₃ alkyl, etc.). Compounds having the structure of formula (III) have the PLG group linked to the ULB through the glutarimide nitrogen. For example, the ULB is lenalidomide derived, pomalidomide derived, or thalidomide derived. In various aspects, the ULB is lenalidomide derived and the compound has the structure of formula (IIIa) or (IIIb):

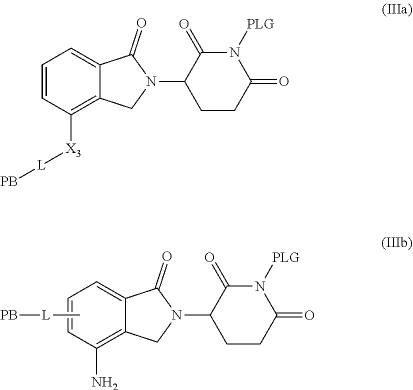

wherein X₃ is —NH— or —O—.

In some embodiments, the ULB moiety is thalidomide derived and the compound has the structure of formula (IIIc):

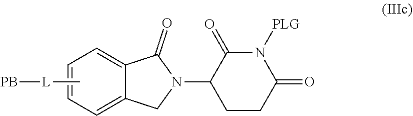

In specific embodiments, ULB moiety is pomalidomide derived and the compound has the structure of formula (IIId) or (IIIe):

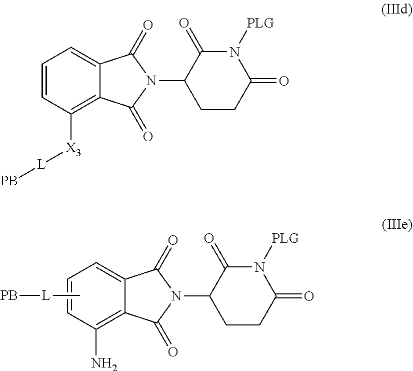

wherein X₃ is —NH— or —O—.

The compound may have the structure of formula (IV):

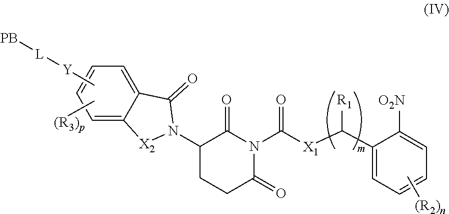

wherein m is 0, 1, or 2;
n is 0, 1, 2, 3, or 4;
p is 0, 1, 2, or 3;
R₁ is independently selected at each occurrence from hydrogen, alkyl (e.g., C₁-C₇ alkyl, C₁-C₃ alkyl, etc.), alkoxy (e.g., C₁-C₇ alkoxy, C₁-C₃ alkoxy, etc.);
R₂ is independently selected at each occurrence from hydrogen, —OC(O)R^{e}, —C(O)OR^{e}, alkyl (e.g., C₁-C₇ alkyl, C₁-C₃ alkyl, etc.), and alkoxy (e.g., C₁-C₇ alkoxy, C₁-C₃ alkoxy, etc.), wherein two vicinal R₂ groups do not together form a ring;
R₃ is independently selected at each occurrence from hydrogen, —N(R^{a})(R^{a}), alkyl (e.g., C₁-C₇ alkyl, C₁-C₃ alkyl, etc.), or alkoxy (e.g., C₁-C₇ alkoxy, C₁-C₃ alkoxy, etc.);
X₁ is —O—, —C(O)—, —NR^{a}—, —OC(O)—, —C(O)O—, —NR^{a}C(O)—, or —C(O)NR^{a}—;
X₂ is C(O), CH₂, C(R^{a})(R^{a}), or NR^{a};
Y is absent (i.e., a bond), —O—, —C(O)—, —OC(O)—, —C(O)O—, —NR^{a}C(O)—, or —C(O)NR^{a}—;
R^{a} is independently selected at each occurrence from hydrogen, or alkyl (e.g., C₁-C₇ alkyl, C₁-C₃ alkyl, etc.); and
R^{e} is independently selected at each occurrence from hydrogen, or alkyl (e.g., C₁-C₇ alkyl, C₁-C₃ alkyl, etc.).

The protein binding moiety may be chosen for a specific protein of interest (POI). Due to the chimeric structure of PROTACs, any compound which binds the POI may be used, such that the protein binding moiety of the compound may be linked (e.g., through a hydrogen position, etc.) to the other portions of the compound (e.g., L, ULB, PLG, etc.) and retain some affinity for the protein of interest. Typically, the POI is degraded following irradiation of the photolabile group and activation of the ULB moiety. In certain embodiments, the protein binding moiety is a protein inhibitor, a protein modulator, or a protein activator. The protein binding moiety may be, for example, a tyrosine kinase inhibitor, a BRAF-mutant inhibitor, or a MEK inhibitor. In some embodiments, the protein binding moiety binds to one or more of Abelson Murine Leukemia (ABL) Proteins (e.g., c-ABL, etc.), Breakpoint Cluster Region Protein (BCR), BCR-ABL fusion proteins, Bromodomain and Extra Terminal Domain (BRD) Family proteins (e.g., BRD2, BRD3, BRDT, BRD4, etc.), anaplastic lymphoma kinase (ALK) protein, echinoderm microtubule-associated protein like, epidermal growth factor (EGFR) family of proteins, and echinoderm microtubule-associated protein like (EML)-ALK fusion proteins.

In certain embodiments, the protein binding moiety may be derived from crizotinib, certinib, or JQ1. For example, the protein binding moiety may be any of the following exemplary derivatives:

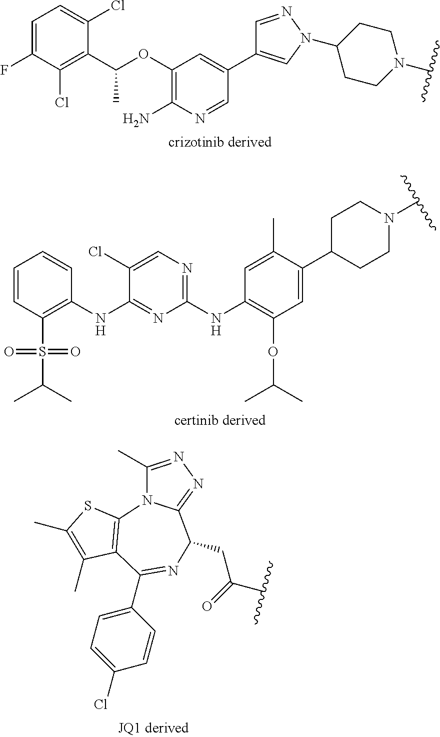 [SMILES: *N1CCC(n2cc(-c3cnc(N)c(O[C@H](C)c4c(Cl)ccc(F)c4Cl)c3)cn2)CC1 ; *N1CCC(c2cc(OC(C)C)c(Nc3ncc(Cl)c(Nc4ccccc4S(=O)(=O)C(C)C)n3)cc2C)CC1 ; *C(=O)C[C@@H]1N=C(c2ccc(Cl)cc2)c2c(sc(C)c2C)-n2c(C)nnc21]

wherein

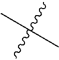

indicates the point of attachment to the L group. In certain embodiments, the protein binding moiety may be derived from (e.g., linked through a hydrogen position of, etc.) Dasatinib, Imatinib, Saracatinib, Ponatinib, Nilotinib, Danusertib, AT9283, Degrasyn, Bafetinib, KW-2449, NVP-BHG712, DCC-2036, GZD824, GNF-2, PD173955, GNF-5, Bosutinib, Gefitinib, Erlotinib, Nivolumab, Sunitinib, Ruxolitinib, Tofacitinib, Lapatinib, Vandetanib, Sorafenib, Sunitinib, Axitinib, Nintedanib, Regorafenib, Pazopanib, Lenvatinib, Crizotinib, Ceritinib, Cabozantinib, DWF, Afatinib, Ibrutinib, B43, KU004, Foretinib, KRCA-0008, PF-06439015, PF-06463922, Canertinib, GSA-10, GW2974, GW583340, WZ4002, CP-380736, D2667, Mubritinib, PD153035, PD168393, Pelitinib, PF-06459988, PF-06672131, PF-6422899, PKI-166, Reveromycin A, Tyrphostin 1, Tyrphostin 23, Tyrphostin 51, Tyrphostin AG 528, Tyrphostin AG 658, Tyrphostin AG 825, Tyrphostin AG 835, Tyrphostin AG 1478, Tyrphostin RG 13022, Tyrphostin RG 14620, B178, GSK1838705A, PD-161570, PD 173074, SU-5402, Roslin 2, Picropodophyllotoxin, PQ401, I-OMe-Tyrphostin AG 538, GNF 5837, GW441756, Tyrphostin AG 879, DMPQ, JNJ-10198409, PLX647, Trapidil, Tyrphostin A9, Tyrphostin AG 370, Lestaurtinib, Geldanamycin, Genistein, GW2580, Herbimycin A, Lavendustin C, Midostaurin, NVP-BHG712, PD158780, PD-166866, PF-06273340, PP2, RPI, SU 11274, SU5614, Symadex, Tyrphostin AG 34, Tyrphostin AG 974, Tyrphostin AG 1007, UNC2881, Honokiol, SU1498, SKLB1002, CP-547632, JK-P3, KRN633, SC-1, ST638, SU 5416, Sulochrin, Tyrphostin SU 1498, 58567, rociletinib, Dacomitinib, Tivantinib, Neratinib, Ramucirumab, Masitinib, Vatalanib, Icotinib, XL-184, OSI-930, AB1010, Quizartinib, AZD9291, Tandutinib, HM61713, Brigantinib, Vemurafenib (PLX-4032), Semaxanib, AZD2171, Crenolanib, Damnacanthal, Fostamatinib, Motesanib, Radotinib, OSI-027, Linsitinib, BIX02189, PF-431396, PND-1186, PF-03814735, PF-431396, sirolimus, temsirolimus, everolimus, deforolimus, zotarolimus, BEZ235, INK128, Omipalisib, AZD8055, MHY1485, PI-103, KU-0063794, ETP-46464, GDC-0349, XL388, WYE-354, WYE-132, GSK1059615, WAY-600, PF-04691502, WYE-687, PP121, BGT226, AZD2014, PP242, CH5132799, P529, GDC-0980, GDC-0994, XMD8-92, Ulixertinib, FR180204, SCH772984, Trametinib, PD184352, PD98059, Selumetinib, PD325901, U0126, Pimasertinib, TAK-733, AZD8330, Binimetinib, PD318088, SL-327, Refametinib, GDC-0623, Cobimetinib, BI-847325, Adaphostin, GNF 2, PPY A, AIM-100, ASP 3026, LFM A13, PF 06465469, (−)-Terreic acid, AG-490, BIBU 1361, BIBX 1382, BMS 599626, CGP 52411, GW 583340, HDS 029, HKI 357, JNJ 28871063, WHI-P 154, PF 431396, PF 573228, FIIN 1, PD 166285, SUN 11602, SR 140333, TCS 359, BMS 536924, NVP ADW 742, PQ 401, BMS 509744, CP 690550, NSC 33994, WHI-P 154, KB SRC 4, DDR1-IN-1, PF 04217903, PHA 665752, SU 16f, A 419259, AZM 475271, PP 1, PP 2, 1-Naphthyl PP1, Src I1, ANA 12, PD 90780, Ki 8751, Ki 20227, ZM 306416, ZM 323881, AEE 788, GTP 14564, PD 180970, R 1530, SU 6668, Toceranib, CEP-32496 (1-(3-((6,7-dimethoxyquinazolin-4-yl)oxy)phenyl)-3-(5-(1,1,1-trifluoro-2-methylpropan-2-yl)isoxazol-3-yl)urea), AZ 628 (4-(2-cyanopropan-2-yl)-N-(4-methyl-3-((3-methyl-4-oxo-3,4-dihydroquinazo- lin-6-yl)amino)phenyl) benzamide), Vemurafenib (PLX-4032), PLX-4720 (N-(3-(5-chloro-1H-pyrrolo[2,3-b]pyridine-3-carbonyl)-2,4-difluorophenyl)-propane-1-sulfonamide), SB 590885 ((E)-5-(2-(4-(2-(dimethylamino)ethoxy)phenyl)-4-(pyridin-4-yl)-1H-imidazo- 1-5-yl)-2,3-dihydro-1H-inden-1-one oxime), and GDC-0879 ((E)-5-(2-(2-hydroxyethyl)-4-(pyridin-4-yl)-1H-imidazol-5-yl)-2,3-dihydro-1H-inden-1-one oxime). In certain implementations, PB is derived from crizotinib, certinib, alectinib, brigatinib, or JQ1.

The functioning of the PROTAC is typically dependent on the binding of the PB moiety to the protein of interest to bring the ULB moiety proximal to the POI following decaging of the photolabile group. In certain embodiments, the PB may have an affinity for its target protein (K_{d}) of less than 1 mM (e.g. from 500 μM to 1 mM, etc.) or less than 500 μM (e.g., less than 450 μM, less than 400 μM, less than 350 μM, less than 300 μM, less than 250 μM, less than 200 μM, less than 150 μM, less than 100 μM, less than 100 μM, less than 50 μM, less than 10 μM, less than 1 μM, less than 500 nm, less than 100 nM, less than 50 nM, less than 10 nM, less than 1 nM, etc.).

The compounds, methods and compositions described herein include the use of N-oxides (if appropriate), crystalline forms (also known as polymorphs), solvates, amorphous phases, and/or pharmaceutically acceptable salts of compounds having the structure of any compound of the disclosure, as well as metabolites and active metabolites of these compounds having the same type of activity. Solvates include water, ether (e.g., tetrahydrofuran, methyl tert-butyl ether, etc.) or alcohol (e.g., ethanol, etc.) solvates, acetates and the like. In certain embodiments, the compounds described herein exist in solvated forms with pharmaceutically acceptable solvents such as water, and ethanol. In other embodiments, the compounds described herein exist in unsolvated form.

In certain embodiments, the compounds of the invention may exist as tautomers or mixtures of tautomers, racemates, mixtures of racemates, stereoisomers, mixtures of stereoisomers, or combinations thereof. All tautomers are included within the scope of the compounds presented herein.

In certain embodiments, compounds described herein are prepared as prodrugs which may be converted into the parent drug in vivo. In certain embodiments, upon in vivo administration, a prodrug is chemically converted to the biologically, pharmaceutically or therapeutically active form of the compound. Typically, prodrugs may be converted into compound capable of binding to the protein of interest, while maintaining the photolabile group. In other embodiments, a prodrug is enzymatically metabolized by one or more steps or processes to the biologically, pharmaceutically or therapeutically active form of the compound.

In some embodiments, the compound has the structure:

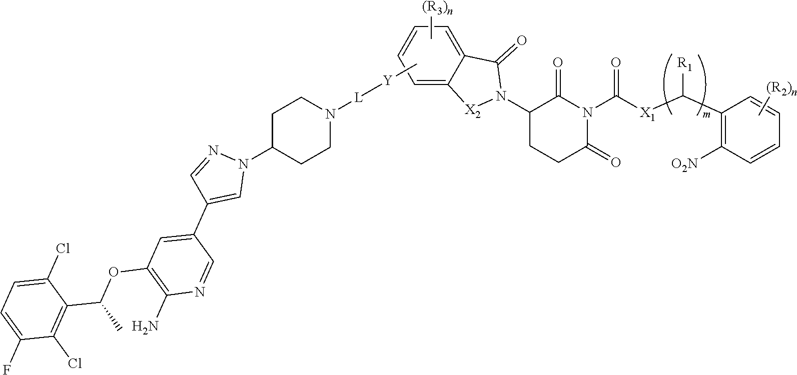

wherein m is 0, 1, or 2;
n is 0, 1, 2, 3, or 4;
p is 1, 2, or, 3;
R₁ is independently selected at each occurrence from hydrogen, alkyl, and alkoxy;
R₂ is independently selected at each occurrence from hydrogen, —OC(O)R^{e}, —C(O)OR^{e}, alkyl, and alkoxy, wherein two vicinal R₂ groups do not together form a ring;
R₃ is independently selected at each occurrence from hydrogen, alkyl, or alkoxy;
X₁ is —O—, —C(O)—, —OC(O)—, —C(O)O—, —NR^{a}C(O)—, or —C(O)NR^{a}—;
X₂ is C(O), CH, CR^{a}, or NR^{a};
Y is a bond, —O—, —C(O)—, —OC(O)—, —C(O)O—, —NR^{a}C(O)—, or —C(O)NR^{a}—;
R^{a} is independently selected at each occurrence from hydrogen, and alkyl; and
R^{e} is independently selected at each occurrence from hydrogen, and alkyl.

In various embodiments, the compound may have the structure:

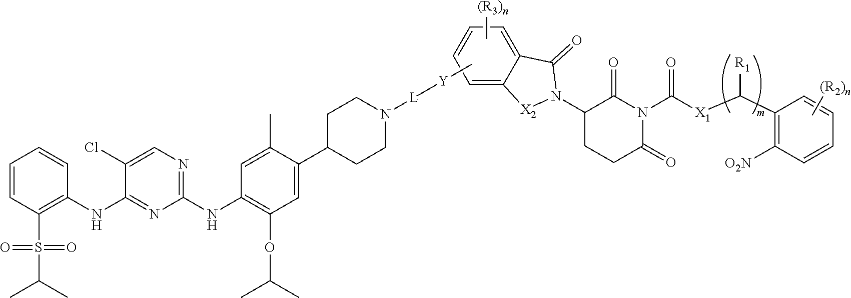

wherein m is 0, 1, or 2;
n is 0, 1, 2, 3, or 4;
p is 1, 2, or, 3;
R₁ is independently selected at each occurrence from hydrogen, alkyl (e.g., C₁-C₇ alkyl, C₁-C₃ alkyl, etc.), alkoxy (e.g., C₁-C₇ alkoxy, C₁-C₃ alkoxy, etc.);
R₂ is independently selected at each occurrence from hydrogen, —OC(O)R^{e}, —C(O)OR^{e}, alkyl (e.g., C₁-C₇ alkyl, C₁-C₃ alkyl, etc.), and alkoxy (e.g., C₁-C₇ alkoxy, C₁-C₃ alkoxy, etc.), wherein two vicinal R₂ groups do not together form a ring;
R₃ is independently selected at each occurrence from hydrogen, alkyl (e.g., C₁-C₇ alkyl, C₁-C₃ alkyl, etc.), or alkoxy (e.g., C₁-C₇ alkoxy, C₁-C₃ alkoxy, etc.);
X₁ is —O—, —C(O)—, —OC(O)—, —C(O)O—, —NR^{a}C(O)—, or —C(O)NR^{a}—;
X₂ is C(O), CH, CR^{a}, or NR^{a};
Y is a bond, —O—, —C(O)—, —OC(O)—, —C(O)O—, —NR^{a}C(O)—, or —C(O)NR^{a}—;
R^{a} is independently selected at each occurrence from hydrogen, or alkyl (e.g., C₁-C₇ alkyl, C₁-C₃ alkyl, etc.); and

R^{e} is independently selected at each occurrence from hydrogen, or alkyl (e.g., C₁-C₇ alkyl, C₁-C₃ alkyl, etc.).

In certain embodiments, the compound has the structure:

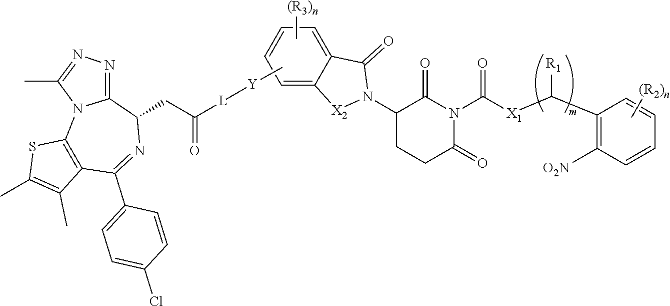

wherein m is 0, 1, or 2;
n is 0, 1, 2, 3, or 4;
p is 1, 2, or, 3;
R₁ is independently selected at each occurrence from hydrogen, alkyl (e.g., C₁-C₇ alkyl, C₁-C₃ alkyl, etc.), alkoxy (e.g., C₁-C₇ alkoxy, C₁-C₃ alkoxy, etc.);
R₂ is independently selected at each occurrence from hydrogen, —OC(O)R^{e}, —C(O)OR^{e}, alkyl (e.g., C₁-C₇ alkyl, C₁-C₃ alkyl, etc.), and alkoxy (e.g., C₁-C₇ alkoxy, C₁-C₃ alkoxy, etc.), wherein two vicinal R₂ groups do not together form a ring;

R₃ is independently selected at each occurrence from hydrogen, alkyl (e.g., C₁-C₇ alkyl, C₁-C₃ alkyl, etc.), or alkoxy (e.g., C₁-C₇ alkoxy, C₁-C₃ alkoxy, etc.);

X₁ is —O—, —C(O)—, —NR^{a}—, —OC(O)—, —C(O)O—, —NR^{a}C(O)—, or —C(O)NR^{a}—;
X₂ is C(O), CH, CR^{a}, or NR^{a};
Y is a bond, —O—, —C(O)—, —NR^{a}—, —OC(O)—, —C(O)O—, —NR^{a}C(O)—, or —C(O)NR^{a}—;
R^{a} is independently selected at each occurrence from hydrogen or alkyl (e.g., C₁-C₇ alkyl, C₁-C₃ alkyl, etc.); and
R^{e} is independently selected at each occurrence from hydrogen or alkyl (e.g., C₁-C₇ alkyl, C₁-C₃ alkyl, etc.).

The linker moiety may be chosen such that the PB moiety is able to bind to the POI and the decaged ULB group is able to degrade the POI following decaging. In some embodiments, L is a divalent hydrocarbon selected from saturated or unsaturated alkylene (e.g., branched alkylelene, linear alkylene, cycloalkylene, C₁-C₂₂ branched alkylelene, C₁-C₂₂ linear alkylene, C₃-C₂₂ cycloalkylene, C₁-C₁₀ branched alkylelene, C₁-C₁₀ linear alkylene, C₃-C₁₀ cycloalkylene, C₁-C₈ branched alkylelene, C₁-C₈ linear alkylene, C₃-C₈ cycloalkylene, etc.), C₁-C₂₂ saturated or unsaturated heteroalkylene (e.g., branched heteroalkylelene, linear heteroalkylene, heterocycloalkylene, C₁-C₂₂ branched heteroalkylelene, C₁-C₂₂ linear heteroalkylene, C₃-C₂₂ heterocycloalkylene, C₁-C₁₀ branched heteroalkylelene, C₁-C₁₀ linear heteroalkylene, C₃-C₁₀ heterocycloalkylene, C₁-C₈ branched heteroalkylelene, C₁-C₈ linear heteroalkylene, C₃-C₈ heterocycloalkylene, etc.), arylene (e.g., C₅-C₂₂ arylene, etc.), heteroarylene (e.g., C₅-C₂₂ heteroarylene, etc.), or combinations thereof. For example, L may comprise one or more of —(C(R^{a})(R^{a}))₁₋₈, —(OC(R^{a})(R^{a}))₁₋₈—, —(OC(R^{a})(R^{a})—C(R^{a})(R^{a}))₁₋₈—, —N(R^{a})—, —O—, or —C(O)—;
wherein R^{a} is independently selected at each occurrence from hydrogen or alkyl (e.g., C₁-C₇ alkyl, C₁-C₄ alkyl, etc.). In some embodiments, L is —(CH₂)₀₋₈—C(O)NH—(CH₂)₀₋₈—, —C(O)NH—(CH₂)₀₋₈—, —NHC(O)—(CH₂)₀₋₈—, —NH—(CH₂)₀₋₈—, or —C(O)—(CH₂)₀₋₈—, or combinations thereof. In specific embodiments, the compound has the structure of formula (Va) or (Vb):

PB—NH—(CH₂)₁₋₈—NH—C(O)—ULB—PLG (Va)

PB—(CH₂)₁₋₈—NH—C(O)—(CH₂)₁₋₈—ULB—PLG (Vb)

The compound may have the structure:

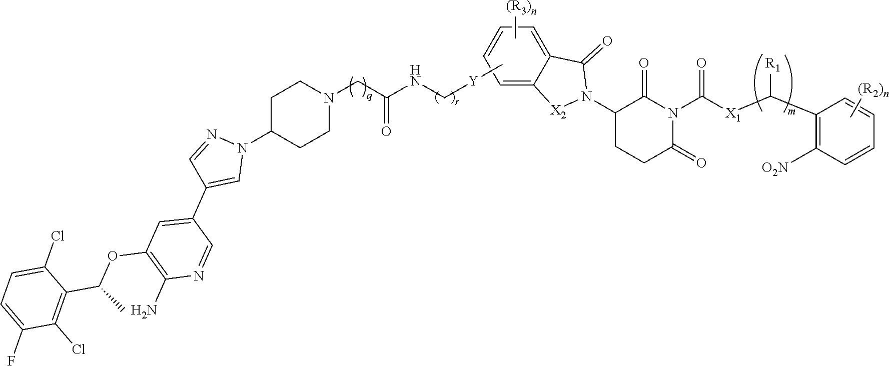

wherein m is 0, 1, or 2;
n is 0, 1, 2, 3, or 4;
p is 1, 2, or, 3;
q and r and independently 0, 1, 2, 3, 4, 5, 6, 7, or 8;
R₁ is independently selected at each occurrence from hydrogen, alkyl (e.g., C₁-C₇ alkyl, C₁-C₃ alkyl, etc.), alkoxy (e.g., C₁-C₇ alkoxy, C₁-C₃ alkoxy, etc.);
R₂ is independently selected at each occurrence from hydrogen, —OC(O)R^{e}, —C(O)OR^{e}, alkyl (e.g., C₁-C₇ alkyl, C₁-C₃ alkyl, etc.), and alkoxy (e.g., C₁-C₇ alkoxy, C₁-C₃ alkoxy, etc.), wherein two vicinal R₂ groups do not together form a ring;
R₃ is independently selected at each occurrence from hydrogen, alkyl (e.g., C₁-C₇ alkyl, C₁-C₃ alkyl, etc.), or alkoxy (e.g., C₁-C₇ alkoxy, C₁-C₃ alkoxy, etc.);
X₂ is C(O), CH, CR^{a}, or NR^{a};
Y is a bond, —O—, —C(O)—, —NR^{a}—, —OC(O)—, —C(O)O—, —NR^{a}C(O)—, or —C(O)NR^{a}—;
R^{a} is independently selected at each occurrence from hydrogen, or alkyl (e.g., C₁-C₇ alkyl, C₁-C₃ alkyl, etc.); and
R^{e} is independently selected at each occurrence from hydrogen, or alkyl (e.g., C₁-C₇ alkyl, C₁-C₃ alkyl, etc.).

In certain embodiments, the compound may have the structure:

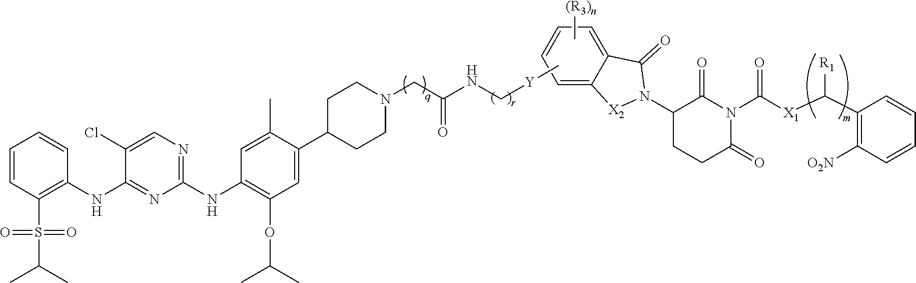

wherein m is 0, 1, or 2;
n is 0, 1, 2, 3, or 4;
p is 1, 2, or, 3;
q and r and independently 0, 1, 2, 3, 4, 5, 6, 7, or 8;
R₁ is independently selected at each occurrence from hydrogen, alkyl (e.g., C₁-C₇ alkyl, C₁-C₃ alkyl, etc.), alkoxy (e.g., C₁-C₇ alkoxy, C₁-C₃ alkoxy, etc.);
R₂ is independently selected at each occurrence from hydrogen, —OC(O)R^{e}, —C(O)OR^{e}, alkyl (e.g., C₁-C₇ alkyl, C₁-C₃ alkyl, etc.), and alkoxy (e.g., C₁-C₇ alkoxy, C₁-C₃ alkoxy, etc.), wherein two vicinal R₂ groups do not together form a ring;
R₃ is independently selected at each occurrence from hydrogen, alkyl (e.g., C₁-C₇ alkyl, C₁-C₃ alkyl, etc.), or alkoxy (e.g., C₁-C₇ alkoxy, C₁-C₃ alkoxy, etc.);
X₂ is C(O), CH, CR^{a} , or NR^{a};
Y is a bond, —O—, —C(O)—, —OC(O)—, —C(O)O—, —NR^{a}C(O)—, or —C(O)NR^{a}—;
R^{a} is independently selected at each occurrence from hydrogen, or alkyl (e.g., C₁-C₇ alkyl, C₁-C₃ alkyl, etc.); and
R^{e} is independently selected at each occurrence from hydrogen, or alkyl (e.g., C₁-C₇ alkyl, C₁-C₃ alkyl, etc.).

In certain embodiments, the compound has the structure:

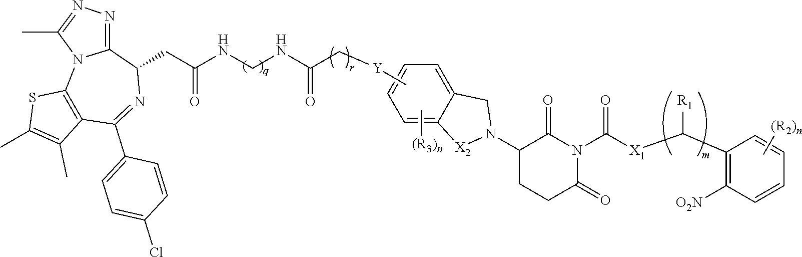

wherein m is 0, 1, or 2;
]n is 0, 1, 2, 3, or 4;
p is 1, 2, or, 3;
R₁ is independently selected at each occurrence from hydrogen, alkyl (e.g., C₁-C₇ alkyl, C₁-C₃ alkyl, etc.), alkoxy (e.g., C₁-C₇ alkoxy, C₁-C₃ alkoxy, etc.);
R₂ is independently selected at each occurrence from hydrogen, —OC(O)R^{e}, —C(O)OR^{e}, alkyl (e.g., C₁-C₇ alkyl, C₁-C₃ alkyl, etc.), and alkoxy (e.g., C₁-C₇ alkoxy, C₁-C₃ alkoxy, etc.), wherein two vicinal R₂ groups do not together form a ring;
R₃ is independently selected at each occurrence from hydrogen, alkyl (e.g., C₁-C₇ alkyl, C₁-C₃ alkyl, etc.), or alkoxy (e.g., C₁-C₇ alkoxy, C₁-C₃ alkoxy, etc.);
X₂ is C(O), CH, CR^{a}, or NR^{a};
Y is a bond, —O—, —C(O)—, —NR^{a}—, —OC(O)—, —C(O)O—, —NR^{a}C(O)—, or —C(O)NR^{a}—;
R^{a} is independently selected at each occurrence from hydrogen or alkyl (e.g., C₁-C₇ alkyl, C₁-C₃ alkyl, etc.); and
R^{e} is independently selected at each occurrence from hydrogen or alkyl (e.g., C₁-C₇ alkyl, C₁-C₃ alkyl, etc.).

In certain embodiments, the compound may be opto-dALK or opto-BET1:

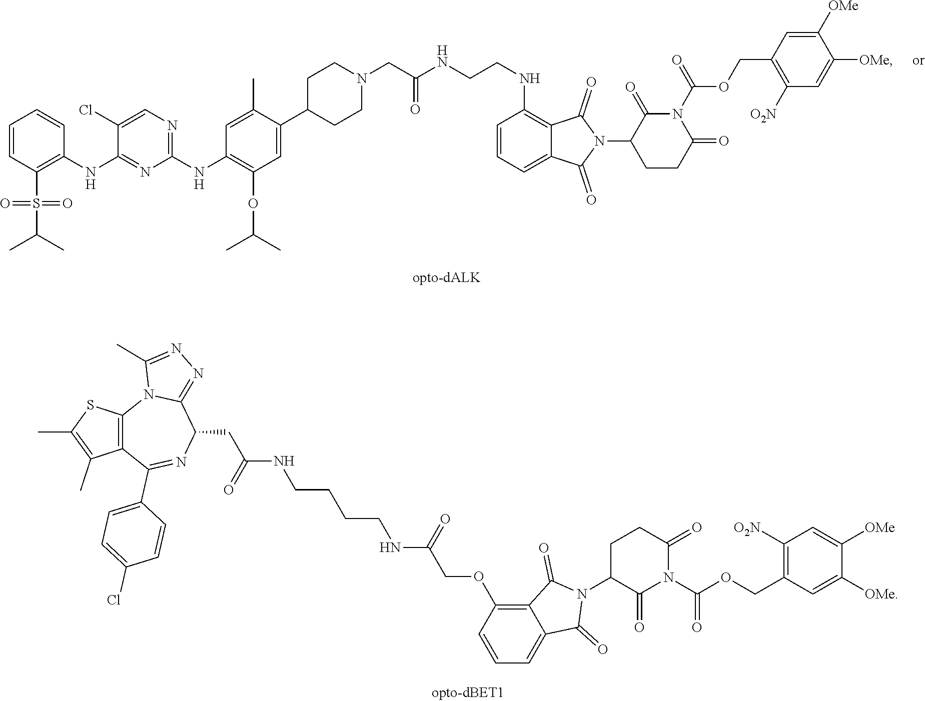 [SMILES: COc1cc(COC(=O)N2C(=O)CCC(N3C(=O)c4cccc(NCCNC(=O)CN5CCC(c6cc(OC(C)C)c(Nc7ncc(Cl)c(Nc8ccccc8S(=O)(=O)C(C)C)n7)cc6C)CC5)c4C3=O)C2=O)c([N+](=O)[O-])cc1OC ; COc1cc(COC(=O)N2C(=O)CCC(N3C(=O)c4cccc(OCC(=O)NCCCCNC(=O)C[C@@H]5N=C(c6ccc(Cl)cc6)c6c(sc(C)c6C)-n6c(C)nnc65)c4C3=O)C2=O)c([N+](=O)[O-])cc1OC]

Non-chimeric molecules are also provided, wherein a ubiquitin binding compound is attached to the photolabile group. For example, the compound may have the structure of formula (VI):

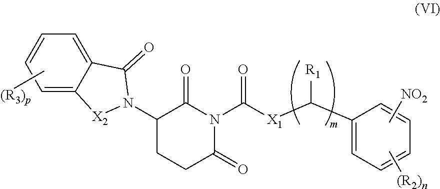

wherein m is 0, 1, or 2;
n is 0, 1, 2, 3, or 4;
p is 0, 1, 2, 3, or 4;
R₁ is independently selected at each occurrence from hydrogen, alkyl (e.g., C₁-C₇ alkyl, C₁-C₃ alkyl, etc.), alkoxy (e.g., C₁-C₇ alkoxy, C₁-C₃ alkoxy, etc.);
R₂ is independently selected at each occurrence from hydrogen, —OC(O)R^{e}, —C(O)OR^{e}, alkyl (e.g., C₁-C₇ alkyl, C₁-C₃ alkyl, etc.), and alkoxy (e.g., C₁-C₇ alkoxy, C₁-C₃ alkoxy, etc.);
R₃ is independently selected at each occurrence from hydrogen, —N(R^{a})(R^{a}), alkyl (e.g., C₁-C₇ alkyl, C₁-C₃ alkyl, etc.), or alkoxy (e.g., C₁-C₇ alkoxy, C₁-C₃ alkoxy, etc.);
X₁ is —O—, —C(O)—, —OC(O)—, —C(O)O—, —NR^{a}C(O)—, or —C(O)NR^{a}—;
X₂ is C(O), CH₂, C(R^{a})(R^{a}), or NR^{a};
R^{a} is independently selected at each occurrence from hydrogen, or alkyl (e.g., C₁-C₇ alkyl, C₁-C₃ alkyl, etc.); and
R^{e} is independently selected at each occurrence from hydrogen, or alkyl (e.g., C₁-C₇ alkyl, C₁-C₃ alkyl, etc.);
or pharmaceutically acceptable salts thereof. In some embodiments, wherein two vicinal R₂ groups do not together form a ring. In some embodiments, the compound has the structure of formula (VIa) (VIb), or (VIc):

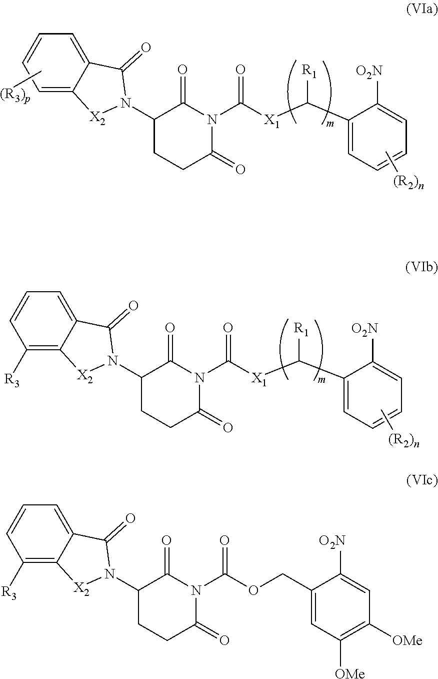

The compound may be, for example, opto-pomalidomide:

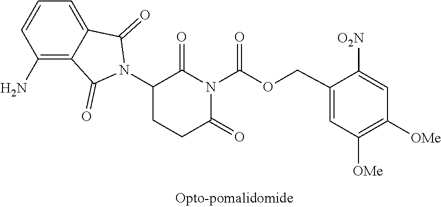 [SMILES: COc1cc(COC(=O)N2C(=O)CCC(N3C(=O)c4cccc(N)c4C3=O)C2=O)c([N+](=O)[O-])cc1OC]

In some embodiments, compounds having the structure of formula (VI) may be used for the synthesis of the PROTACs described herein (e.g., compounds having the structure of formula (I), formula (III)).

The disclosure includes a pharmaceutical composition comprising at least one compound described herein (e.g., compounds having the structure of formula (I), compounds having the structure of formula (III), compounds having the structure of formula (VI), etc.) and at least one pharmaceutically acceptable carrier, diluent, or diluent. In certain embodiments, the composition is formulated for an administration route such as oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

The pharmaceutical composition may comprise a compound disclosed herein (e.g., compounds having the structure of formula (I), compounds having the structure of formula (III), compounds having the structure of formula (IV), etc.) and one or more pharmaceutically acceptable salts, carriers, or diluents. In specific embodiments, the compound is formulated as a topical composition (e.g., ointment, gel, etc.). In some embodiments, the composition comprises from 0.1%-90% (e.g., 0.1%-50%, 0.1%-20%, 0.1%-10%, etc.) of the compound by weight of the composition.

The disclosure includes a method of treating or preventing a disease associated with and/or caused by overexpression and/or uncontrolled activation of certain proteins (e.g., tyrosine kinase) in a subject in need thereof. The invention further includes a method of treating or preventing a cancer associated with and/or caused by a specific protein of interest (e.g., an oncogenic tyrosine kinase, etc.) in a subject in need thereof In certain embodiments, the disease comprises a cancer. In some implementations, the tyrosine kinase is c-ABL and/or BCR-ABL. In yet other embodiments, the cancer is chronic myelogenous leukemia (CML).

Examples of cancers that can be treated or prevented by the present invention include but are not limited to: squamous cell cancer, lung cancer including small cell lung cancer, non-small cell lung cancer, vulval cancer, thyroid cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, hepatic carcinoma, anal carcinoma, penile carcinoma, and head and neck cancer. In certain embodiments, the cancer is at least one selected from the group consisting of ALL, T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitts Lymphoma, B-cell ALL, Philadelphia chromosome positive ALL, Philadelphia chromosome positive CML, lymphoma, leukemia, multiple myeloma, myeloproliferative diseases, large B cell lymphoma, and B cell Lymphoma. In certain embodiments, the proliferative disease is melanoma, leukemia, lymphoma, or retinal blastoma.

The methods of the disclosure may comprise administering to the subject a therapeutically effective amount of at least one compound of the invention, which is optionally formulated in a pharmaceutical composition. In certain embodiments, the method further comprises administering to the subject an additional therapeutic agent that treats or prevents cancer. The compound may be a compound for the treatment of a hyperproliferative disease. In certain embodiments, the compound may be a compound for the manufacture of a medicament for the treatment of a hyperproliferative disease. The method for the treatment of a proliferative disease may comprise the administration of a compound or pharmaceutical composition as disclosed herein.

In order to decage the compounds, the method may further comprise irradiating the patient with electromagnetic radiation comprising photons of one or more wavelengths and a power density for an irradiation time period sufficient to induce the separation of the photolabile group from the ubiquitin ligase binding moiety of the compound. In some embodiments, the photons have one or more wavelengths between 300 and 450 nm. In certain implementations, the electromagnetic radiation has a wavelength spectrum with a maximum at one or more wavelengths between 300 and 450 nm. In some embodiments, the electromagnetic radiation has a wavelength spectrum with a maximum between 325-375 nm.

The patient may be irradiated with light only at the location where the disease is localized. In some embodiments, the proliferative disease is localized in a specific area of the patient, the compound is administered to one or more portions of the specific area, and the electromagnetic radiation is irradiated to one or more portions of the specific area. For example, the proliferative disease may be located on the skin, eye, blood, mouth (e.g., gums, etc.), throat, esophagus, digestive tract, or colon, of the patient.

In certain embodiments, the compounds are stable in the absence of light. Without wishing to be bound by theory, how the photolabile group is conjugated may be implicated in the stability. The carbamate linkage may be able to enhace the stability of these compounds when the compound has not been exposed to electromagnetic radiation. Only upon exposure to certain forms of electromagnetic radiation will the photolabile group be removed.

The removal of the photolabile group to induce ubiquitinization of the POI (e.g., via the irradiation step, etc.) may occur at some time period following administration of the compounds disclosed herein. In some embodiments, the time period between the administration and the irradiation is a length sufficient to induce binding between the compound and the cells of the proliferative disease on the patient. In various implementations, the patient is not exposed to radiation capable of separation of the photolabile group and the ubiquitin ligase binding moiety during administration and/or during the time period. In certain embodiments, the time period between administration and irradiation is more than 5 minutes (e.g., more than 10 minutes, more than 20 minutes, more than 30 minutes, more than an hour, more than 6 hours, more than 12 hours, more than a day, etc.). In some embodiments, the irradiation time period is more than 60 seconds (e.g., more than 120 seconds, more than 180 seconds, etc.). In some embodiments, at least one portion of the specific area is irradiated for more than 30 seconds (e.g., more than 60 seconds, more than 120 seconds, more than 180 seconds, more than 5 minutes, from 1 minute and 15 minutes, from 4 minutes to 15 minutes, etc.). In various implementations, two or more portions are irradiated sequentially. In some embodiments, two or more portions are irradiated for an independently selected irradiation time period based on the characteristics of the portion (e.g., proliferative disease density, type, etc.). In some embodiments, the irradiation area is moved around an area of the subject where the compounds disclosed herein have been applied, such that the rate of irradiation area movement allows decaging of the compounds applied.

The electromagnetic radiation has the characteristics required for decaging of the compounds disclosed herein. For example, the electromagnetic radiation may have a spot size on the patient of from 0.1 mm² to 100 cm² (e.g., from 0.1 mm² to 1000 mm², from 1000 mm² to 0.1 cm², from 0.1 cm² to 10 cm² from 10 cm² to 100 cm², etc.). In certain embodiments, the electromagnetic light is monochromatic radiation having a spectral bandwidth of less than 50 nm or less than 10 nm (e.g., less than 5 nm, less than 1 nm, etc.). For example, the electromagnetic radiation may be monochromatic light with a wavelength from 300 to 400 nm (e.g., from 300 to 310 nm, from 310 nm to 320 nm, from 320 nm to 330 nm, from 330 nm to 340 nm, from 340 nm to 350 nm, from 350 nm to 360 nm, from 360 nm to 370 nm, from 370 nm to 380 nm, from 380 nm to 390 nm, from 390 nm to 400, nm, 365 nm etc.). In certain embodiments, the electromagnetic radiation may have a power density of from 0.1 mW/cm² to 1000 mW/cm² (e.g., from 0.1 mW/cm² to 1 mW/cm², from 1 mW/cm² to 10 mW/cm², from 10 mW/cm² to 100 mW/cm², 100 mW/cm² to 1000 mW/cm², etc.).

In certain embodiments, the separation of the photolabile group from the ubiquitin ligase binding moiety of the compound may occurs following exposure to environmental light (e.g., sunlight, etc.), particularly when the compound is administered to the the skin of the subject.

In certain embodiments, administering the compound of the disclosure to the subject allows for administering a lower dose of the additional therapeutic agent as compared to the dose of the additional therapeutic agent alone that is required to achieve similar results in treating or preventing a cancer in the subject. For example, in certain embodiments, the compounds disclosed herein may enhance the anti-cancer activity of the additional therapeutic compound, thereby allowing for a lower dose of the additional therapeutic compound to provide the same effect. In certain embodiments, the compounds and the therapeutic agent are co-administered to the subject. In other embodiments, the compound of the invention and the therapeutic agent are coformulated and co-administered to the subject.

In certain embodiments, the subject is a mammal. In other embodiments, the mammal is a human.

The therapeutically effective amount or dose of a compound of the present invention depends on the age, sex and weight of the patient, the current medical condition of the patient and the progression of a cancer in the patient being treated. The skilled artisan is able to determine appropriate dosages depending on these and other factors. For example, the therapeutically effective amount may be determined based on the amount of decaged photolabile compound required to treat a proliferative disease of interest. For example, the therapeutically effective amount may be based on more than 20% or more than 30% or more than 40% or more than 50% or more than 60% or more than 70% or more than 80% or more than 90% or more than 95% or more than 95% or more than 99% or 100% irradiative decaging of the compounds described herein by weight of the composition.

For example, a suitable dose of a compound of the present invention may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses. Additionally, the electromagnetic radiation may be altered to achieve the required dose. For example, in some embodiments, the administered area may be irradiated with a power density of from 0.1 mW/cm² to 1000 mW/cm² (e.g., from 0.1 mW/cm² to 1 mW/cm², from 1 mW/cm² to 10 mW/cm², from 10 mW/cm² to 100 mW/cm², 100 mW/cm² to 1000 mW/cm², etc.).

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the inhibitor of the invention is optionally given continuously; alternatively, the dose of drug being administered is temporarily reduced or temporarily suspended for a certain length of time. The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced to a level at which the improved disease is retained. In certain embodiments, patients require intermittent treatment on a long-term basis upon any recurrence of symptoms and/or infection.

The compounds for use in the method of the invention may be formulated in unit dosage form. Typically, unit dosage forms are physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD50 (the dose lethal to 50% of the population) and the ED5o (the dose therapeutically effective in 50% of the population). The dose ratio between the toxic and therapeutic effects is the therapeutic index, which is expressed as the ratio between LD50 and ED5o. The data obtained from cell culture assays and animal studies are optionally used in formulating a range of dosage for use in human. The dosage of such compounds lies preferably within a range of circulating concentrations that include the ED5o with minimal toxicity. The dosage optionally varies within this range depending upon the dosage form employed and the route of administration utilized.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

### EXAMPLES

The following examples illustrate specific aspects of the instant description. The examples should not be construed as limiting, as the example merely provides specific understanding and practice of the embodiments and its various aspects.

Materials for Experimentation

Plasmids and Chemicals

Flag-CRBN and HA-IKZF1 were provided by Dr. William G. Kaelin (Dana-Farber Cancer Institute). GFP-BRD2 and GFP-BRD3 were purchased from Addgene. dBET1 was obtained from Dr. J. E. Bradner's group at the Dana-Farber Cancer Institute. Pomalidomide was purchased from Sigma. dALK was synthesized as described in C. Zhang et al., Eur J Med Chem 151 (2018): 304, hereby incorporated by reference in its entirety and specifically in relation to synthetic schemes of ALK PROTACs.

Cell Culture

Human embryonic kidney 293T (HEK293T) cells and HEK293FT were maintained in Dulbecco's Modified Eagle's Medium (DMEM) containing 10% fetal bovine serum (FBS), 100 Units of penicillin and 100 pg/m1 streptomycin. MM.1S, MT2, C4-2, NCI-H2228 and NCI-H3122 cells were cultured in RPMI1640 containing 10% fetal bovine serum (FBS), 100 Units of penicillin and 100 μg/ml streptomycin. 293FT^{CRBN+/+}, 293FT^{CRBN+/+} , MM.1S^{CRBN+/+} and MM.1S^{CRBN-/-} cells were provided Dr. William G. Kaelin (Dana-Farber Cancer Institute). For UVA irradiation, cells were pretreated with opto-PROTACs for 2-4 hours, and then subjected to UVA irradiation for indicated durations.

Antibodies

Anti-IKZF1 (ab191394) antibody was purchased from Abcam. Anti-IKZF3 (NBP22449) antibody was purchased from Novus Biologicals. Anti-BRD3 (11859-1-AP) antibody was purchased from Proteintech. Anti-BRD4 (A301-985A-M) antibody was purchased from Bethyl Laboratories. Anti-ALK (3633) was purchased from Cell Signaling Technologies. Monoclonal anti-HA antibody (MMS-101P) was purchased from BioLegend. Polyclonal anti-HA (sc-805) antibody was purchased from Santa Cruz. Anti-GFP antibody (632380) was purchased from Invitrogen. Polyclonal anti-Flag antibody (F-2425), monoclonal anti-Flag antibody (F-3165, clone M2), anti-tubulin antibody (T-5168), anti-vinculin antibody (V-4505), anti-Flag agarose beads (A-2220), anti-HA agarose beads (A-2095), peroxidase-conjugated anti-mouse secondary antibody (A-4416) and peroxidase-conjugated anti-rabbit secondary antibody (A-4914) were purchased from Sigma. All antibodies were used at a 1:1,000 dilution in 5% bovine serum albumin (BSA) in Tris buffered saline with Tween 20 (TBST) buffer for western blots.

General Experimental Procedures

The following experimental protocols describe the general methods used in the Examples described below.

Chemistry Methods

HPLC spectra were acquired using an Agilent 1200 Series system with DAD detector for all the intermediates and Opto-PROTACs below. Chromatography was performed on a 2.1×150 mm Zorbax 300SB-C18 5 μm column with water containing 0.1% formic acid as solvent A and acetonitrile containing 0.1% formic acid as solvent B at a flow rate of 0.4 ml/min. The gradient program was as follows: 1% B (0-1 min), 1-99% B (1-4 min), and 99% B (4-8 min). High-resolution mass spectra (FIRMS) data were acquired in positive ion mode using an Agilent G1969A API-TOF with an electrospray ionization (ESI) source. Nuclear Magnetic Resonance (NMR) spectra were acquired on a Bruker DRX-600 spectrometer with 600 MHz for proton (¹H NMR) and 151 MHz for carbon (¹³C NMR); chemical shifts are reported in (δ). Preparative HPLC was performed on Agilent Prep 1200 series with UV detector set to 254 nm. Samples were injected onto a Phenomenex Luna 250×30 mm, 5 μm, C₁₈ column at room temperature. The flow rate was 40 ml/min. A linear gradient was used with 10% of acetonitrile (A) in H₂O (with 0.1% TFA) (B) to 100% of acetonitrile (A). HPLC was used to establish the purity of target compounds. All final compounds had >95% purity using the HPLC methods described above.

UV-VIS Absorption Spectrum

UV-Vis spectrometry was performed on a NanoDrop-2000 UV-Visible Spectrophotometer. Pomalidomide, opto-pomalidomide, dBET1, opto-dBET1, dALK and opto-dALK were dissolved in DMSO and diluted to indicated concentration, followed by UVA irradiation for indicated duration of time. Then the samples were subjected to UV-Vis spectrometry analysis. Pomalidomide and opto-pomalidomide mixture solutions were made with the indicated ratio. Absorption values at 364 nm were measured to draw standard curve. dBET1 and opto-dBET1 mixture solution were made with the indicated ratios. Absorption values at 340 nm were measured to draw standard curve. dALK and opto-dALK mixture solution were made with indicated ratio. Absorption values at 370 nm were measured to draw standard curve.

Immunoblots (IB) and Immunoprecipitation (IP)

Cells were lysed in EBC lysis buffer (50 mM Tris pH 7.5, 120 mM NaCl, 0.5% NP-40), supplemented with protease inhibitors (cOmplete Mini, Roche) and phosphatase inhibitors (phosphatase inhibitor cocktail set I and II, Calbiochem). The protein concentrations of the lysates were measured using the Bio-Rad protein assay reagent on a Beckman Coulter DU-800 spectrophotometer. The lysates were then resolved by sodium dodecylsulfate-polyacrylamide gel ectrophoresis (SDS-PAGE) and immunoblotted with indicated antibodies. For immunoprecipitation, 1 mg lysates were incubated with the appropriate sepharose beads for 4 h at 4° C. Immuno-complexes were washed four times with NETN lysis buffer (20 mM Tris, pH 8.0, 100 mM NaCl, 1 mM EDTA and 0.5% NP-40) before being resolved by SDS-PAGE and immunoblotted with indicated antibodies.

In Vitro Pull-Down Assays

Flag-CRBN was expressed in HEK293T cells lysed in PROTAC buffer B (50 mM Tris pH 7.5, 150 mM NaCl, 0.5% NP-40) supplemented with protease inhibitors (cOmplete Mini, Roche) and phosphatase inhibitors (phosphatase inhibitor cocktail set I and II, Calbiochem). 3mg cell lysis was incubated with 10 μl 10mM biotin-pomalidomide and 8 streptavidin beads for 1 h at 4° C. in the absence or presence of pomalidomide or opto-pomalidomide. Then, the beads were washed four times with NETN buffer (20 mM Tris, pH 8.0, 100 mM NaCl, 1 mM EDTA and 0.5% NP-40) before being resolved by SDS-PAGE and immunoblotted with indicated antibodies.

In Vivo Ubiquitination Assays

Denatured in vivo ubiquitination assays were performed as described in H. Inuzuka et al., Cell 150 (2012): 179, hereby incorporated by reference in its entirety and specifically in relation to ubiquitination assays. Briefly, HEK293T cells were transfected with Flag-CRBN, His-ubiquitin and HA-IKZF1 or GFP-BRD2 or GFP-BRD3. 24 hours after transfection, pomalidomide, opto-pomalidomide, dBET1 or opto-dBET1 were added to the cell culture together with 10 μM MG132 for 12 hours and cells were harvested in denatured buffer A (6 M guanidine-HCl, pH 8.0, 0.1 M Na₂HPO₄/NaH₂PO₄, 10 mM imidazole). After sonication, the ubiquitinated proteins were purified by incubation with Ni-NTA matrices for 3 hours at room temperature. The pull-down products were washed sequentially twice in buffer A, twice in buffer ANTI mixture (buffer A: buffer TI=1:3) and once in buffer TI (25 mM Tris-HCl, pH 6.8, 20 mM imidazole). The poly-ubiquitinated proteins were separated by SDS-PAGE for immunoblot analysis.

CCK-8 Cell Proliferation Assay

Cell in 96-well plates were incubated with 10 ul/well of CCK-8 solution and incubated for 2 hours, followed by the measurement of optical density using a microplate reader with a 450 nm filter (O.D. 450).

Statistical Analysis

The quantitative data from multiple repeat experiments were analyzed by a two-tailed unpaired Student's t test or one-way ANOVA, and presented as mean±S.E.M. When P<0.05, the data were considered as statistically significant.

### Example 1

### Synthesis of Opto-dBET1

### 4,5-dimethoxy-2-nitrobenzyl 3-(4-amino-1,3-dioxoisoindolin-2-yl)-2,6-dioxopiperidine-1-carboxylate (Opto-pomalidomide)

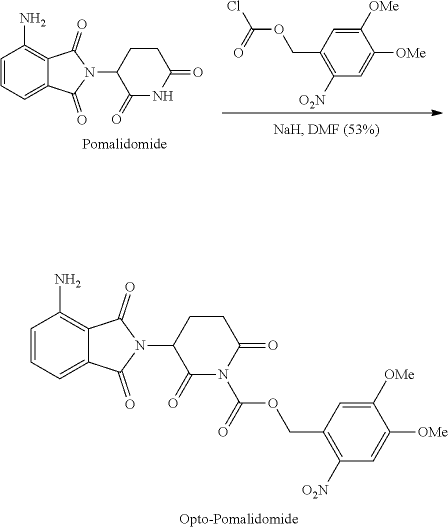 [SMILES: Nc1cccc2c1C(=O)N(C1CCC(=O)NC1=O)C2=O ; COc1cc(COC(=O)Cl)c([N+](=O)[O-])cc1OC ; COc1cc(COC(=O)N2C(=O)CCC(N3C(=O)c4cccc(N)c4C3=O)C2=O)c([N+](=O)[O-])cc1OC]

To a solution of pomalidomide (27 mg, 0.1 mmol, 1.0 equiv) in dimethylformaminde (DMF) (1 mL) was added NaH (4.8 mg, 60% in mineral oil, 0.12 mmol, 1.2 equiv) at 0° C. After stirring for 10 min, 4,5-dimethoxy-2-nitrobenzyl carbonochloridate (33 mg, 0.12 mmol, 1.2 equiv) was added to the mixture at 0° C. The reaction mixture was then warmed up to room temperature and stirred for additional 3 h. The resulting mixture was purified by preparative HPLC (10%-100% acetonitrile/0.1% TFA in H₂O) to afford Opto-pomalidomide as yellow solid (27.0 mg, 53%). ¹H NMR (600 MHz, DMSO-d₆) δ 7.74 (s, 1H), 7.49 (dd, J=8.5, 7.0 Hz, 1H), 7.26 (s, 1H), 7.04 (d, J=8.6 Hz, 1H), 7.02 (d, J=7.1 Hz, 1H), 6.58 (s, 2H), 5.81 (d, J=14.5 Hz, 1H), 5.77 (d, J=14.4 Hz, 1H), 5.40 (dd, J=12.9, 5.5 Hz, 1H), 3.88 (s, 3H), 3.86 (s, 3H), 3.14 (ddd, J=17.4, 13.8, 5.5 Hz, 1H), 2.86 (ddd, J=17.4, 4.4, 2.6 Hz, 1H), 2.63 (qd, J=13.3, 4.4 Hz, 1H), 2.18-2.11 (m, 1H).¹³C NMR (151 MHz, DMSO-d₆) δ 170.8, 168.6, 168.6, 167.6, 153.8, 150.8, 148.5, 147.3, 139.6, 136.0, 132.2, 125.3, 122.3, 111.5, 110.9, 108.6, 67.5, 56.7, 56.5, 48.7, 31.2, 21.8. ESI m/z=535.2 [M+Na⁺]. HRMS calcd for C₂₃H₂₄N₅O₁₀⁺ [M+NH₄⁺] 530.1518, found 530.1536.

tert-butyl (4-(2-((2-(2,6-dioxopiperidin-3-yl)-1,3-dioxoisoindolin-4-yl)oxy) acetamido) butyl)carbamate was synthesized as described in G. E. Winter et al., Science 348 (2015): 1376, hereby incorporated by reference in its entirety, and specifically in relation to synthesis of tert-butyl (4-(2-((2-(2,6-dioxopiperidin-3-yl)-1,3-dioxoisoindolin-4-yl)oxy) acetamido) butyl)carbamate.

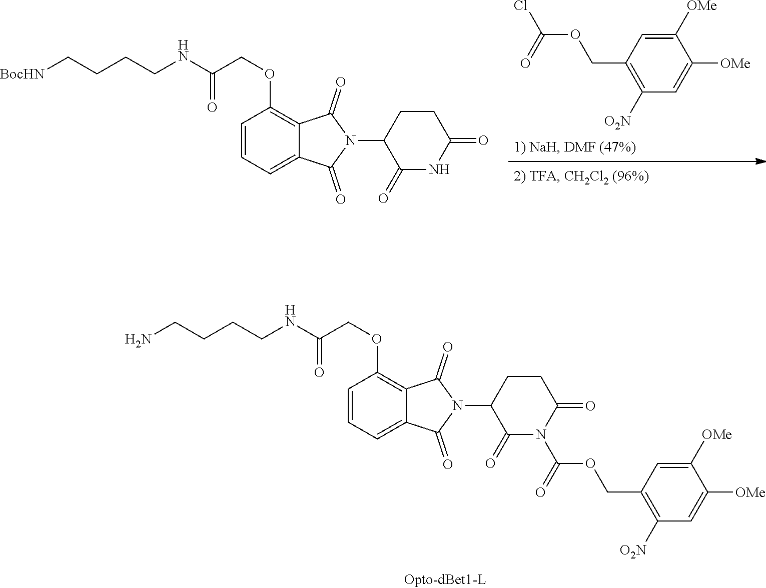 [SMILES: CC(C)(C)OC(=O)NCCCCNC(=O)COc1cccc2c1C(=O)N(C1CCC(=O)NC1=O)C2=O ; COc1cc(COC(=O)Cl)c([N+](=O)[O-])cc1OC ; COc1cc(COC(=O)N2C(=O)CCC(N3C(=O)c4cccc(OCC(=O)NCCCCN)c4C3=O)C2=O)c([N+](=O)[O-])cc1OC]

To a solution of tent-butyl (4-(2-((2-(2,6-dioxopiperidin-3-yl)-1,3-dioxoisoindolin-4-yl)oxy)acetamido)butyl)carbamate (50 mg, 0.1 mmol, 1.0 equiv) in DMF (1 mL) was added NaH (4.8 mg, 60% in mineral oil, 0.12 mmol, 1.2 equiv) at 0° C. After stirring for 10 min, 4,5-dimethoxy-2-nitrobenzyl carbonochloridate (33 mg, 0.12 mmol, 1.2 equiv) was added to the mixture at 0° C. The reaction mixture was then warmed upto room temperature and stirred for additional 3 h. The resulting mixture was purified by preparative HPLC (10%-100% acetonitrile/0.1% TFA in H₂O) to afford desired product as white solid (34.6 mg, 47%). ESI m/z=642.3 [M−Boc+H⁺]. HRMS calcd for C₃₄H₃₉N₅O₁₄Na⁺ [M+Na⁺] 764.2386, found 764.2400.

To a solution of obtained above compound (34.6 mg, 0.047 mmol, 1.0 equiv) in CH₂Cl₂ (2 mL) was added trifluoroacetic acid (TFA) (1 mL) at room temperature. After stirring for 1 h, the resulting mixture was purified by preparative HPLC (10%-100% acetonitrile/0.1% TFA in H₂O) to afford Opto-dBET1-L as white solid in TFA salt form (34.0 mg, 96%). ¹H NMR (600 MHz, DMSO-d₆) δ 8.08 (t, J=5.9 Hz, 1H), 7.85 (dd, J=8.5, 7.3 Hz, 1H), 7.75 (s, 1H), 7.72 (s, 3H), 7.53 (d, J=7.2 Hz, 1H), 7.41 (d, J=8.6 Hz, 1H), 7.27 (s, 1H), 5.81 (d, J=14.4 Hz, 1H), 5.77 (d, J=14.4 Hz, 1H), 5.47 (dd, J=12.8, 5.5 Hz, 1H), 4.80 (s, 2H), 3.89 (s, 3H), 3.87 (s, 3H), 3.19-3.12 (m, 3H), 2.88 (ddd, J=17.5, 4.4, 2.7 Hz, 1H), 2.80 (h, J=6.0 Hz, 2H), 2.63 (qd, J=13.2, 4.4 Hz, 1H), 2.19-2.12 (m, 1H), 1.57-1.45 (m, 4H). ¹³C NMR (151 MHz, DMSO-d₆) δ 170.7, 168.3, 167.2, 166.9, 165.6, 155.6, 153.8, 150.8, 148.5, 139.6, 137.5, 133.3, 125.2, 120.9, 117.0, 116.6, 111.0, 108.7, 68.0, 67.6, 56.7, 56.6, 49.0, 38.9, 38.1, 31.2, 26.4, 24.8, 21.6. ESI m/z =642.3 [M+H⁺]. HRMS calcd for C₂₉H₃₂N₅O₁₂⁺[M+H⁺] 642.2042, found 642.2036.

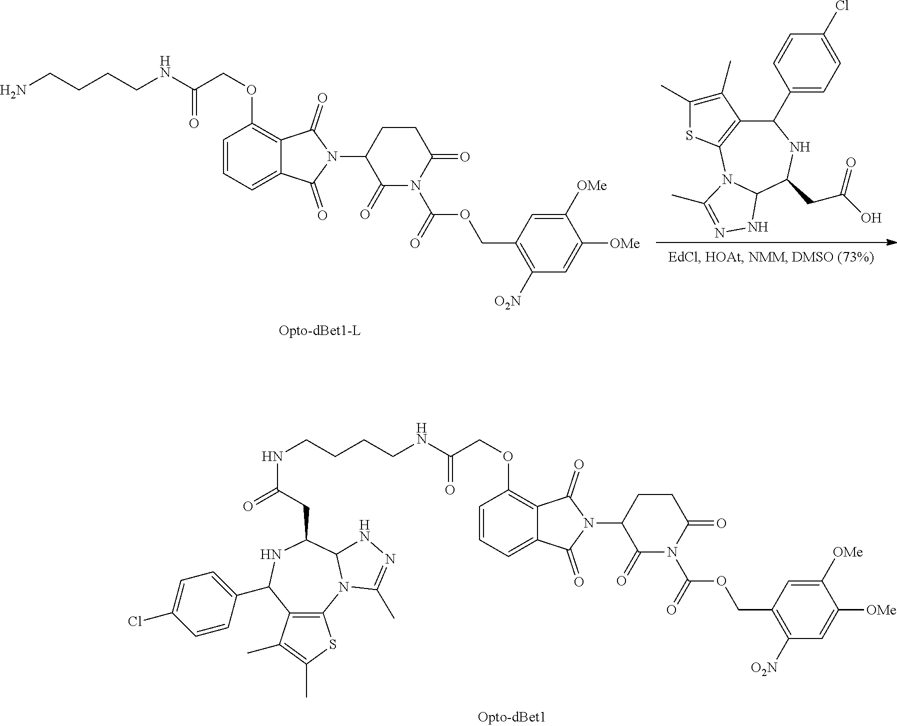 [SMILES: COc1cc(COC(=O)N2C(=O)CCC(N3C(=O)c4cccc(OCC(=O)NCCCCN)c4C3=O)C2=O)c([N+](=O)[O-])cc1OC ; CC1=NNC2[C@H](CC(=O)O)NC(c3ccc(Cl)cc3)c3c(sc(C)c3C)N12 ; COc1cc(COC(=O)N2C(=O)CCC(N3C(=O)c4cccc(OCC(=O)NCCCCNC(=O)C[C@@H]5NC(c6ccc(Cl)cc6)c6c(sc(C)c6C)N6C(C)=NNC56)c4C3=O)C2=O)c([N+](=O)[O-])cc1OC]

To a solution of Opto-dBET1-L (23.0 mg, 0.03 mmol, 1.1 equiv) in DMSO (1 mL) were added (S)-2-(4-(4-chlorophenyl)-2,3,9-trimethyl-6H-thieno[3,2-f][1,2,4]triazolo[4,3-a][1,4]diazepin-6-yl)acetic acid (see reference 7 for the details of synthesis) (14.4 mg, 0.028 mmol, 1 equiv), EDCI (1-ethyl-3-(3-dimethylaminopropyl)carbodiimide) (8.1 mg, 0.042 mmol, 1.5 equiv), HOAt (1-hydroxy-7-azabenzo-triazole) (5.7 mg, 0.042 mmol, 1.5 equiv), and NMM (N-Methylmorpholine) (14.2 mg, 0.14 mmol, 5.0 equiv). After being stirred overnight at room temperature, the resulting mixture was purified by preparative HPLC (10%-100% acetonitrile/0.1% TFA in H₂O) to afford Opto-dBET1 as light yellow solid in TFA salt form (23.2 mg, 73%). ¹H NMR (600 MHz, DMSO-d₆) δ 8.21 (t, J=5.7 Hz, 1H), 8.02 (t, J=5.8 Hz, 1H), 7.83 (dd, J=8.5, 7.3 Hz, 1H), 7.74 (s, 1H), 7.53-7.46 (m, 3H), 7.45-7.39 (m, 3H), 7.26 (s, 1H), 5.81 (d, J=14.4 Hz, 1H), 5.77 (d, J=14.4 Hz, 1H), 5.47 (dd, J=12.9, 5.4 Hz, 1H), 4.80 (s, 2H), 4.52 (dd, J=8.1, 6.1 Hz, 1H), 3.88 (s, 3H), 3.86 (s, 3H), 3.29-3.05 (m, 7H), 2.92-2.82 (m, 1H), 2.71-2.57 (m, 4H), 2.41 (s, 3H), 2.20-2.11 (m, 1H), 1.62 (s, 3H), 1.54-1.43 (m, 4H). ¹³C NMR (151 MHz, DMSO-d₆) δ 170.7, 169.8, 168.3, 167.1, 166.9, 165.6, 163.5, 155.6, 155.5, 153.8, 150.8, 150.3, 148.5, 139.6, 137.5, 137.1, 135.8, 133.3, 132.6, 131.2, 130.5, 130.3, 130.0, 128.9, 125.3, 120.8, 117.0, 116.5, 110.9, 108.7, 68.0, 67.5, 56.7, 56.5, 54.2, 49.0, 38.5, 38.5, 38.0, 31.2, 27.0, 26.9, 21.6, 13.1, 11.7. ESI m/z=1024.3 [M+H⁺] HRMS calcd for C₄₈H₄₇N₉O₁₃SCl⁺ [M+H⁺] 1024.2697, found 1024.2734.

### Example 2

### Synthesis of Opto-dALK

tert-butyl (2-((2-(2,6-dioxopiperidin-3-yl)-1,3-dioxoisoindolin-4-yl)amino)ethyl) carbamate was synthesized as described in C. Zhang et al., Eur J Med Chem 151 (2018): 304, hereby incorporated by reference in its entirety and specifically in relation to synthetic schemes of tert-butyl (2-((2-(2,6-dioxopiperidin-3-yl)-1,3-dioxoisoindolin-4-yl)amino) ethyl) carbamate.

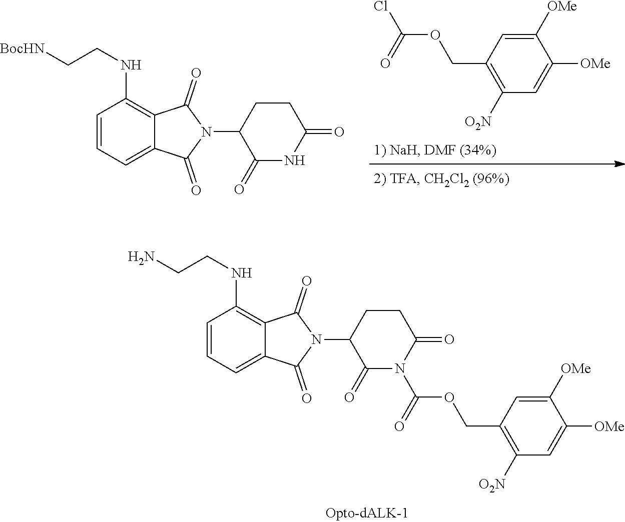 [SMILES: CC(C)(C)OC(=O)NCCNc1cccc2c1C(=O)N(C1CCC(=O)NC1=O)C2=O ; COc1cc(COC(=O)Cl)c([N+](=O)[O-])cc1OC ; COc1cc(COC(=O)N2C(=O)CCC(N3C(=O)c4cccc(NCCN)c4C3=O)C2=O)c([N+](=O)[O-])cc1OC]

To a solution of tent-butyl (2-((2-(2,6-dioxopiperidin-3-yl)-1,3-dioxoisoindolin-4-yl)amino)ethyl)carbamate (20.0 mg, 0.048 mmol, 1.0 equiv) in DMF (1 mL) was added NaH (2.9 mg, 60% in mineral oil, 0.072 mmol, 1.5 equiv) at 0° C. After stirring for 10 min, 4,5-dimethoxy-2-nitrobenzyl carbonochloridate (16.0 mg, 0.058 mmol, 1.2 equiv) was added to the mixture at 0° C. The reaction mixture was warmed to room temperature and stirred for additional 3 h. The resulting mixture was purified by preparative HPLC (10%-100% acetonitrile/0.1% TFA in H₂O) to afford desired product as yellow solid (10.7 mg, 34%). ESI m/z=556.2 [M−Boc+H⁺]. HRMS calcd for C₂₅H₂₆N₅O₁₀⁺ [M−Boc+H⁺] 556.1674, found 556.1690.

To a solution of obtained above compound (10.7 mg, 0.016 mmol, 1.0 equiv) in CH₂Cl₂ (2 mL) was added TFA (1 mL) at room temperature. After stirring for 1 h, the resulting mixture was purified by preparative HPLC (10%-100% acetonitrile / 0.1% TFA in H₂O) to afford Opto-dALK-L as yellow solid in TFA salt form (10.6 mg, 96%). ¹H NMR (600 MHz, Methanol-d₄) δ 7.78 (s, 1H), 7.66 (dd, J=8.6, 7.1 Hz, 1H), 7.28 (s, 1H), 7.21-7.16 (m, 2H), 5.85 (d, J=15.2 Hz, 1H), 5.76 (d, J=15.3 Hz, 1H), 5.32 (dd, J=12.8, 5.5 Hz, 1H), 3.92 (s, 3H), 3.90 (s, 3H), 3.71 (t, J=6.1 Hz, 2H), 3.22 (t, J=6.1 Hz, 2H), 3.08 (ddd, J=17.6, 13.7, 5.3 Hz, 1H), 2.98 (ddd, J=17.6, 4.5, 2.8 Hz, 1H), 2.83 (qd, J=13.2, 4.4 Hz, 1H), 2.24-2.17 (m, 1H). ¹³C NMR (151 MHz, Methanol-d₄) δ 170.0, 168.8, 167.9, 167.4, 154.1, 150.3, 148.3, 146.0, 139.0, 136.2, 132.5, 125.6, 116.5, 111.6, 110.9, 109.4, 107.8, 67.1, 55.8, 55.3, 48.8, 39.5, 38.2, 30.8, 21.6. ESI m/z=556.2 [M+H⁺]. HRMS calcd for C₂₅H₂₆N₅O₁₀⁺ [M+H⁺] 556.1674, found 556.1712.

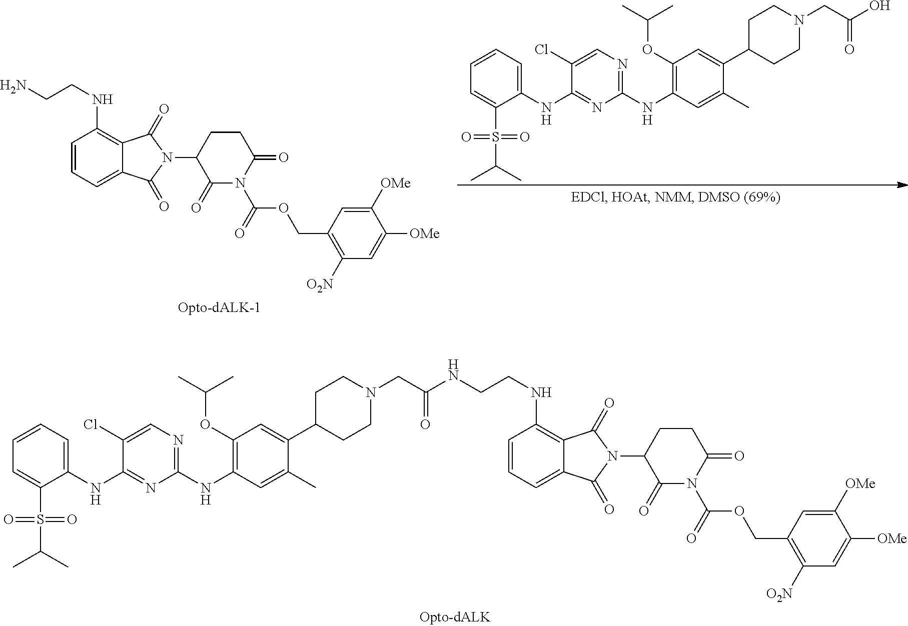 [SMILES: COc1cc(COC(=O)N2C(=O)CCC(N3C(=O)c4cccc(NCCN)c4C3=O)C2=O)c([N+](=O)[O-])cc1OC ; Cc1cc(Nc2ncc(Cl)c(Nc3ccccc3S(=O)(=O)C(C)C)n2)c(OC(C)C)cc1C1CCN(CC(=O)O)CC1 ; COc1cc(COC(=O)N2C(=O)CCC(N3C(=O)c4cccc(NCCNC(=O)CN5CCC(c6cc(OC(C)C)c(Nc7ncc(Cl)c(Nc8ccccc8S(=O)(=O)C(C)C)n7)cc6C)CC5)c4C3=O)C2=O)c([N+](=O)[O-])cc1OC]

To a solution of Opto-dALK-L (8.1 mg, 0.012 mmol, 1.1 equiv) in DMSO (1 mL) were added 69%) (see reference 17 for the details of synthesis) (8.3 mg, 0.011 mmol, 1.0 equiv), EDCI (1-ethyl-3-(3-dimethylaminopropyl)carbodiimide) (3.2 mg, 0.017 mmol, 1.5 equiv), HOAt (1-hydroxy-7-azabenzo-triazole) (2.3 mg, 0.017 mmol, 1.5 equiv), and NMM (N-Methylmorpholine) (5.6 mg, 0.055 mmol, 5.0 equiv). After being stirred overnight at room temperature, the resulting mixture was purified by preparative HPLC (10%-100% acetonitrile/0.1% TFA in H₂O) to afford Opto-dALK as yellow solid in TFA salt form (10.0 mg, 69%).¹H NMR (600 MHz, Methanol-d₄) δ 8.34 (d, J=8.3 Hz, 1H), 8.23 (s, 1H), 7.98 (d, J=7.9 Hz, 1H), 7.72 (d, J=6.0 Hz, 1H), 7.66-7.57 (m, 3H), 7.46 (t, J=7.7 Hz, 1H), 7.19 (d, J=8.6 Hz, 1H), 7.11 (d, J=7.1 Hz, 1H), 7.07 (s, 1H), 6.79 (s, 1H), 5.61 (d, J=15.3 Hz, 1H), 5.49 (d, J=15.3 Hz, 1H), 5.28 (dd, J=12.9, 5.4 Hz, 1H), 4.59 (p, J=6.0 Hz, 1H), 3.94 (s, 2H), 3.87 (s, 3H), 3.82 (s, 3H), 3.69-3.51 (m, 6H), 3.42 (p, J=6.8 Hz, 1H), 3.23-3.13 (m, 2H), 3.08-3.01 (m, 2H), 2.93 (ddd, J=17.5, 4.4, 2.8 Hz, 1H), 2.83-2.74 (m, 1H), 2.22 -2.14 (m, 1H), 2.12 (s, 3H), 2.07-1.93 (m, 4H), 1.33 (d, J=6.0 Hz, 6H), 1.28 (d, J=6.8 Hz, 6H). ¹³C NMR (151 MHz, Methanol-d₄) δ 169.9, 169.0, 167.9, 167.4, 164.4, 156.5, 154.8, 154.0, 150.2, 148.1, 146.7, 146.2, 138.6, 137.2, 137.0, 136.1, 134.8, 132.3, 127.2, 126.6, 125.9, 125.6, 124.7, 122.7, 116.9, 110.9, 110.5, 109.8, 108.9, 107.6, 105.3, 71.3, 67.1, 57.1, 55.8, 55.3, 53.7, 48.7, 41.5, 37.9, 34.7, 30.7, 29.5, 29.4, 21.5, 21.2, 20.9, 20.6, 17.6, 14.4, 14.1, 13.7. ESI m/z=1153.5 [M+H⁺]. HRMS calcd for C₅₅H₆₂N₁₀O₁₇SCl⁺ [M+H⁺] 1153.3851, found 1153.3848.

### Example 3

### Opto-Pomalidomide Decaging

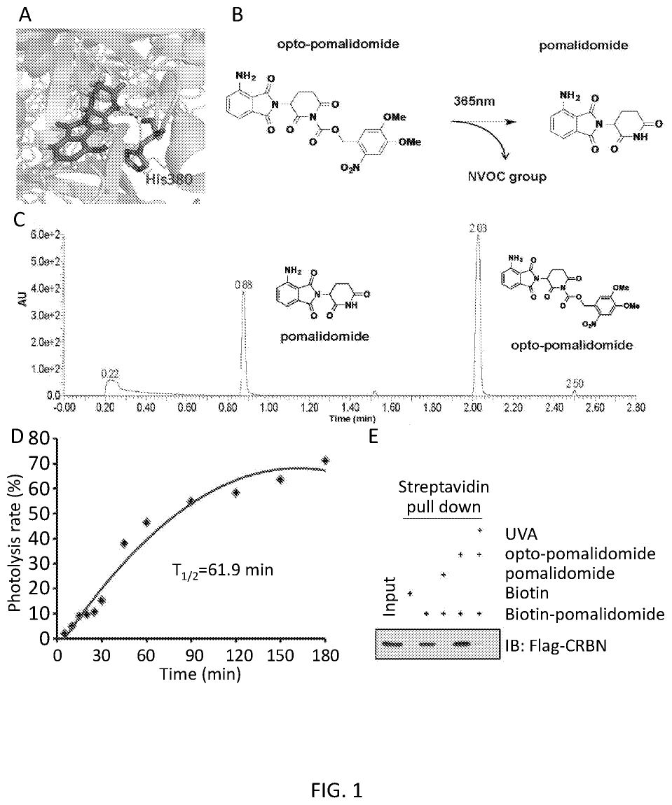

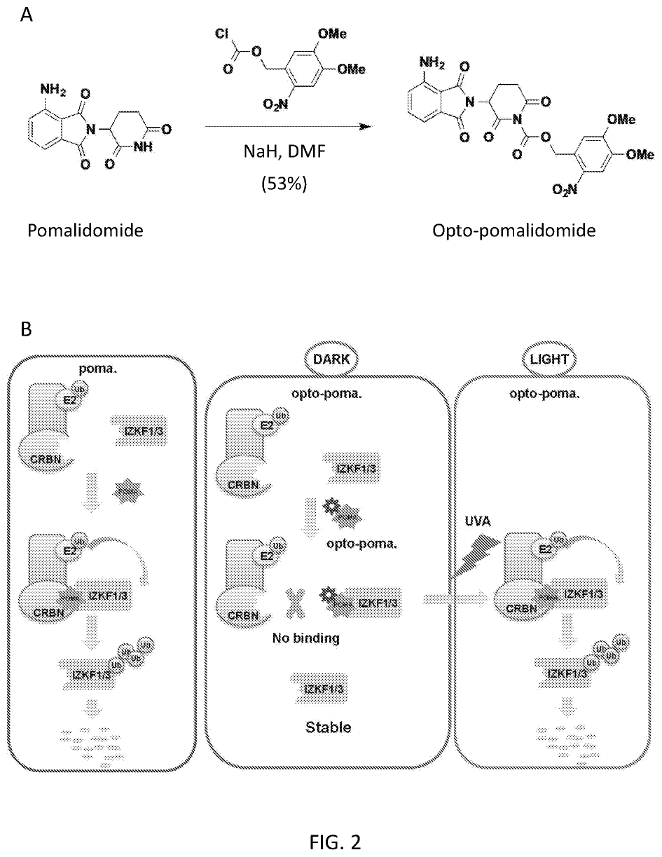

Light-inducible proteolysis targeting chimeras (PROTACs), herein referred to as opto-PROTACs, were synthesized by addition of a photolabile caging group on pomalidomide. The photolablile caging group was able to block the pomalidomide interaction with the E3 ligase Cereblon (CRBN). Given that the key hydrogen bond is formed between glutarimide NH of pomalidomide and the backbone carbonyl of His380 in CRBN as shown in J. Lu et al., Chem Biol 22 (2015): 755, E. S. Fischer et al., Nature 512 (2014): 49, each hereby incorporated by reference in their entirety. As shown in FIG. 2A, the photolabile nitroveratryloxycarbonyl (NVOC) group was installed on the glutarimide nitrogen of pomalidomide and analyzed for decaging effects. A schematic of the decaging mechanism is illustrated in FIG. 2B. This engineered opto-pomalidomide molecule could be induced to undergo photolysis by UVA irradiation in vitro (FIGS. 1B-C, 3A-B, 4A-B), in a time dependent manner (FIG. 1D, 5A-E).

Biotin-pomalidomide was used to pull down Flag-CRBN purified from HEK293T cells, with or without indicated drug (pomalidomide or opto-pomalidomide with/without UVA irradiation). Biotin was used as a negative control. In contrast to free pomalidomide, the inert opto-pomalidomide was ineffective to bind with CRBN in vitro (FIG. 1E), while UVA irradiation efficiently uncaged it from the caged status to be functionally activated, as demonstrated in its regained ability to bind with CRBN.

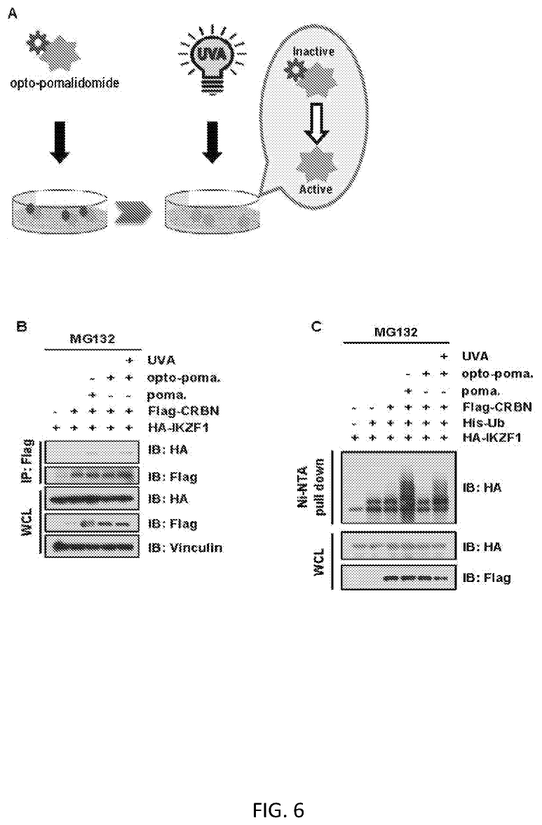

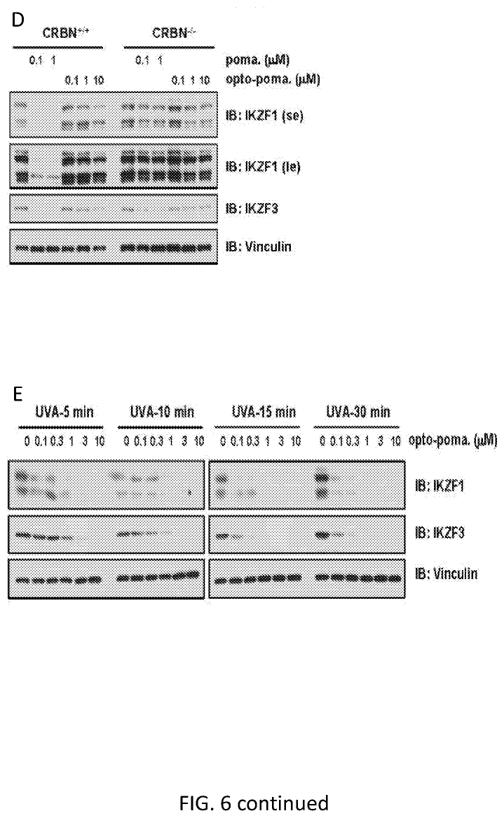

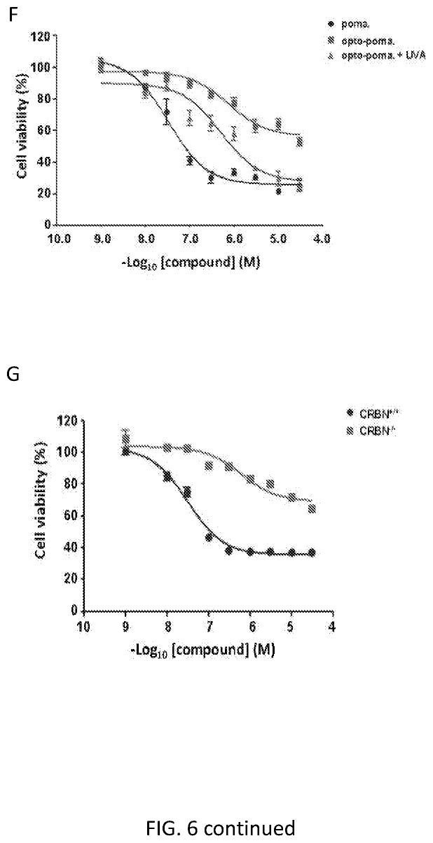

HEK293T cells were pretreated with opto-pomalidomide and the binding between CRBN and IKZF1 was determined as shown in FIG. 6A. As can be seen, pomalidomide bound both CRBN and IKZF1/3 to subsequently transfer the ubiquitin chain onto the target proteins, IKZFs (FIGS. 6B-C). In contrast, opto-pomalidomide was inert as it was ineffective in guiding the protein-protein interaction between CRBN and IKZF1 (FIG. 6B). After activation by UVA irradiation, the uncaged opto-pomalidomide (pomalidomide) regained the ability to promote the binding between CRBN and IKZF 1, thus leading to CRBN-mediated IKZF1 ubiquitination (FIG. 6C). Moreover, pomalidomide induced IKZF1/3 degradation in a CRBN-dependent manner, while opto-pomalidomide lost this function without UVA irradiation. This loss occurs even with 10-folds of excess opto-drug concentration (FIG. 6D).

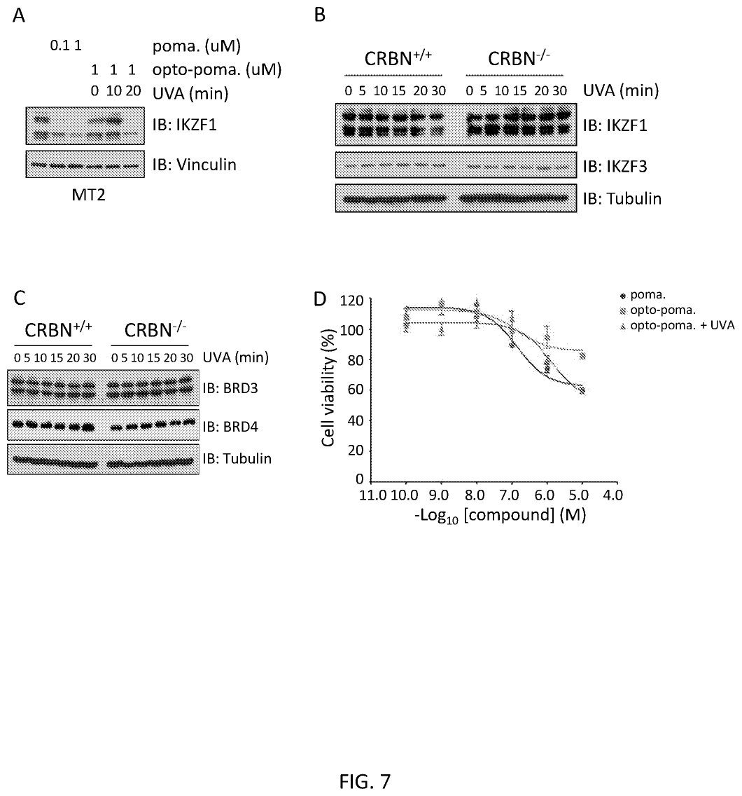

Opto-pomalidomide-pretreated cells were stimulated with different durations of UVA irradiation (FIG. 6E). As can be seen, the observed degradation of IKZF1/3 by opto-pomalidomide occurred in a drug-dose and UVA-dose dependent manner (FIGS. 6E, 7A). More importantly, this degradation was not induced by UVA irradiation itself when opto-pomalidomide was not present (FIGS. 7B-C), further demonstrating the specific regulation of IKZFs degradation by the engineered opto-pomalidomide in a light-dependent manner. Moreover, as a biological consequence, the ability of opto-pomalidomide to kill multiple myeloma was largely dependent on UVA irradiation (FIG. 6F-G, 7D).

### Example 4

### Opto-PROTAC Analysis-Opto-dBET1

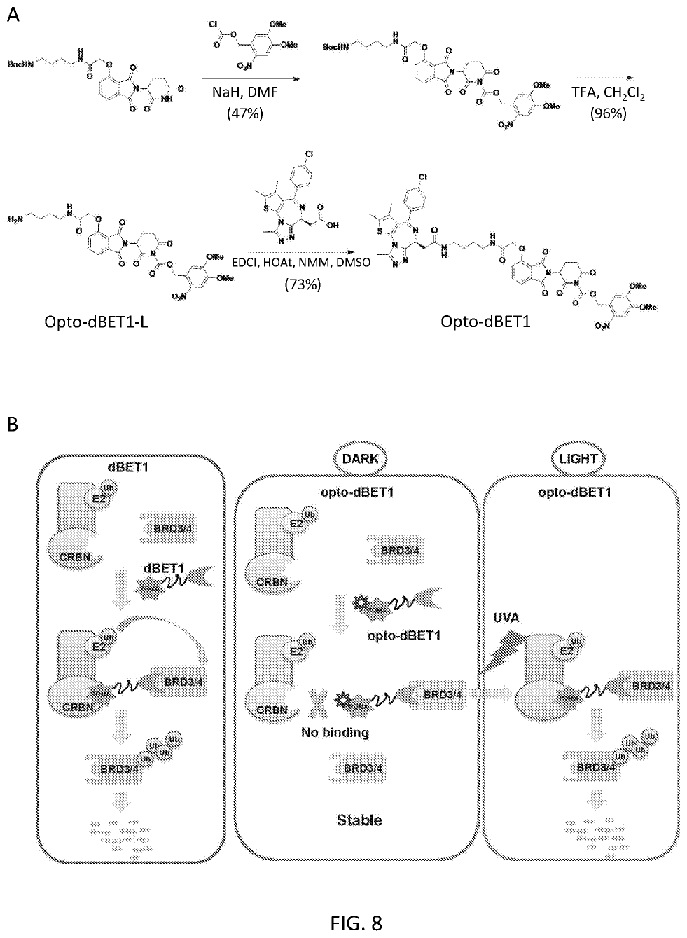

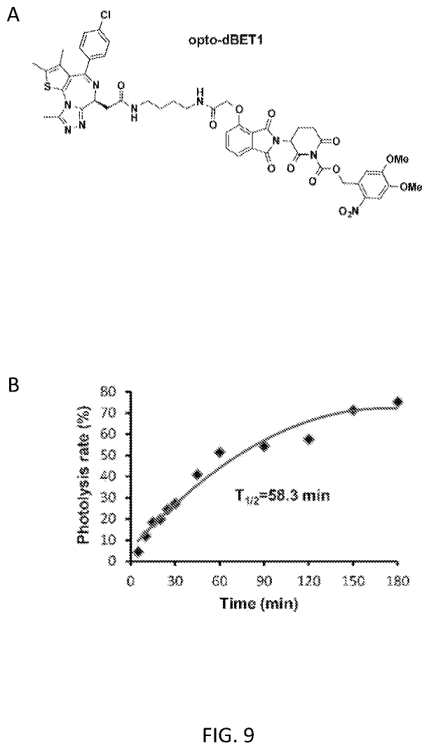

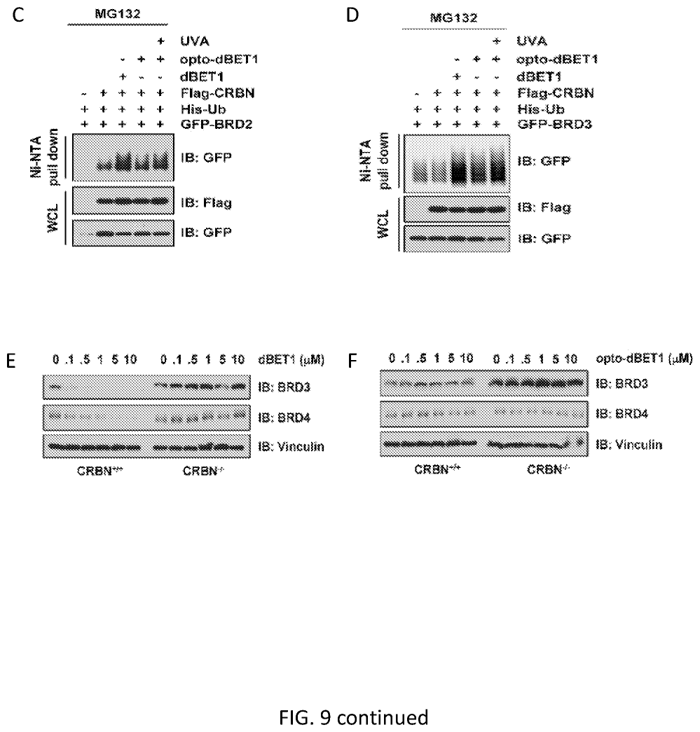

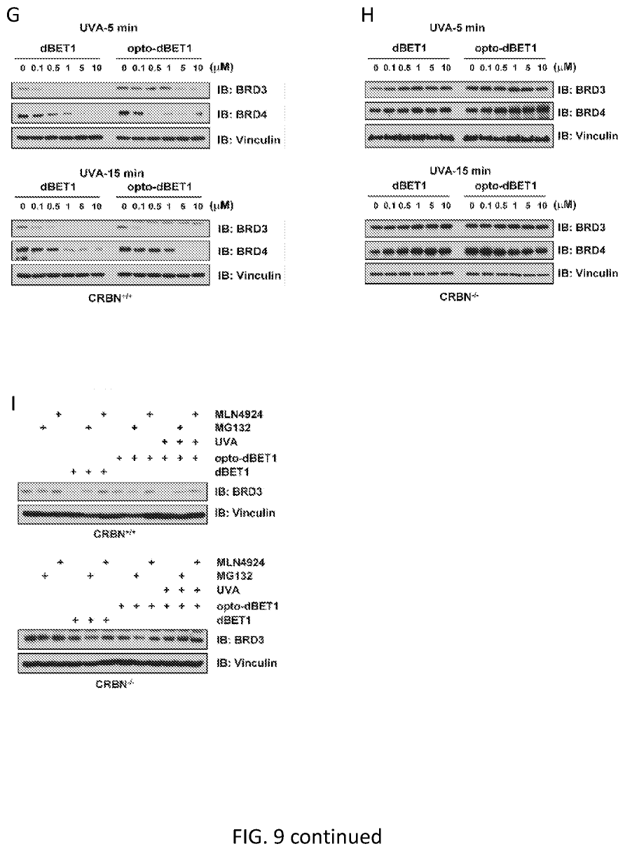

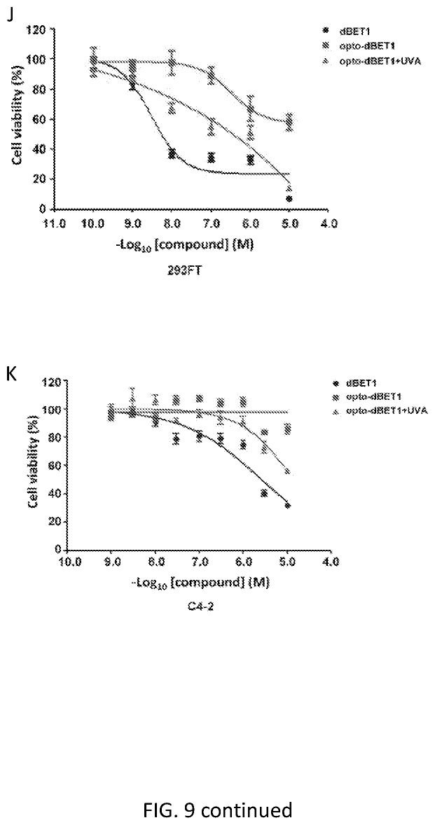

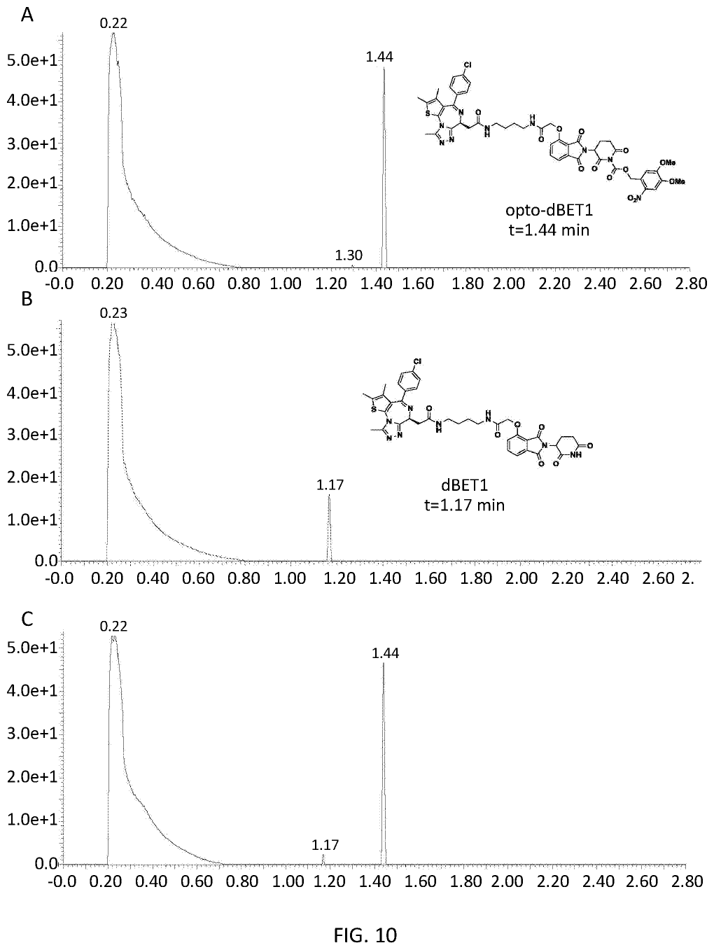

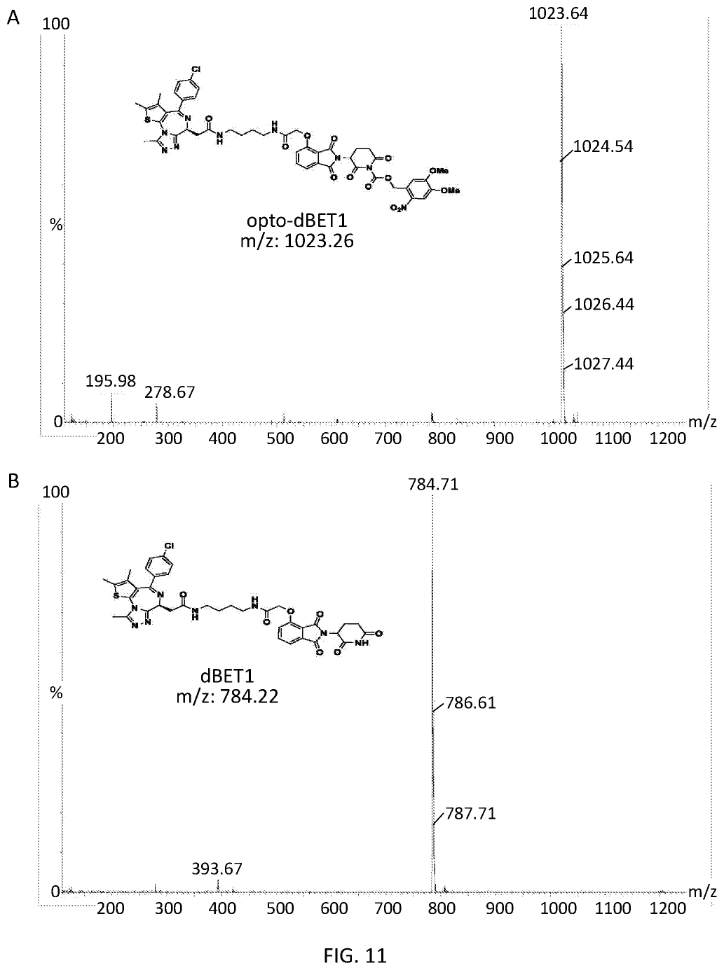

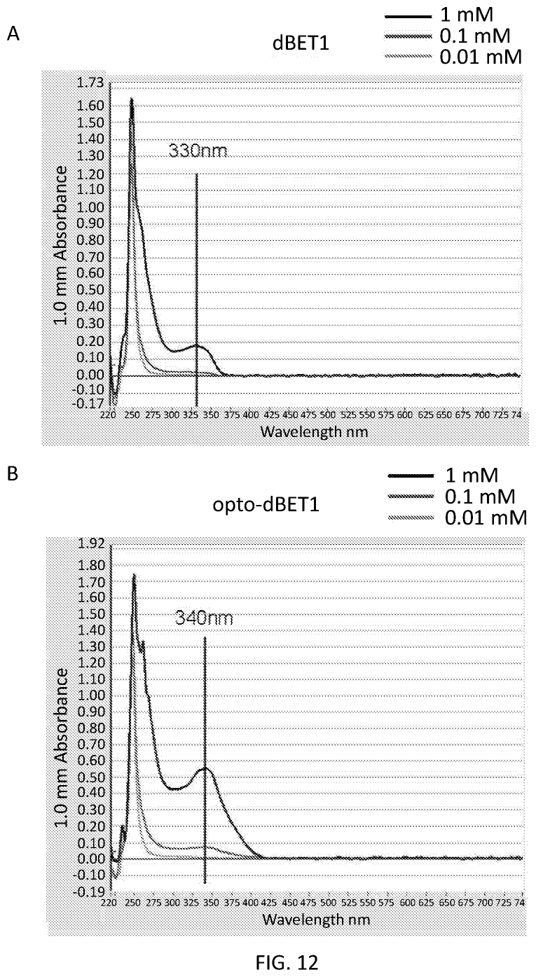

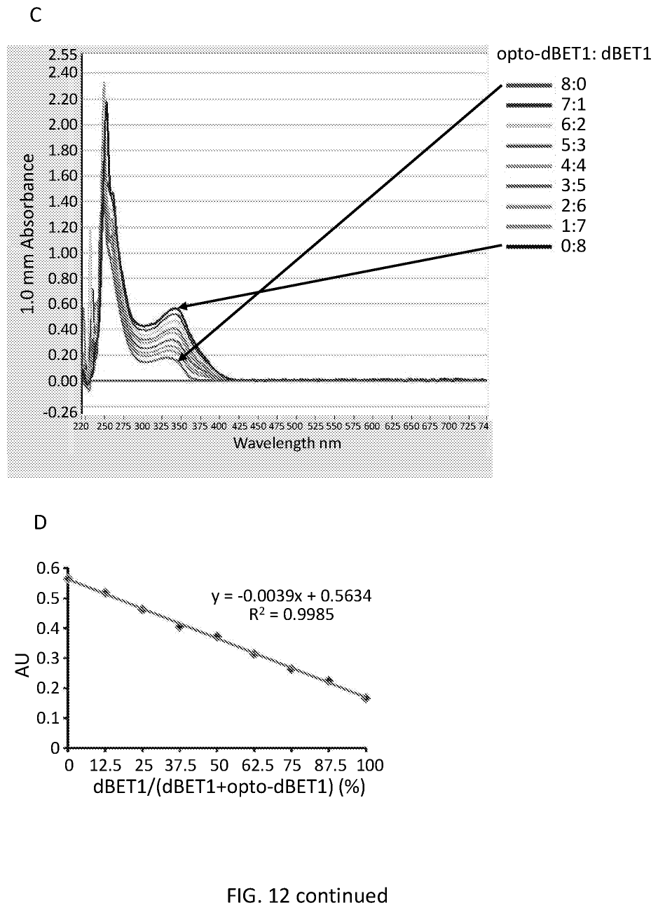

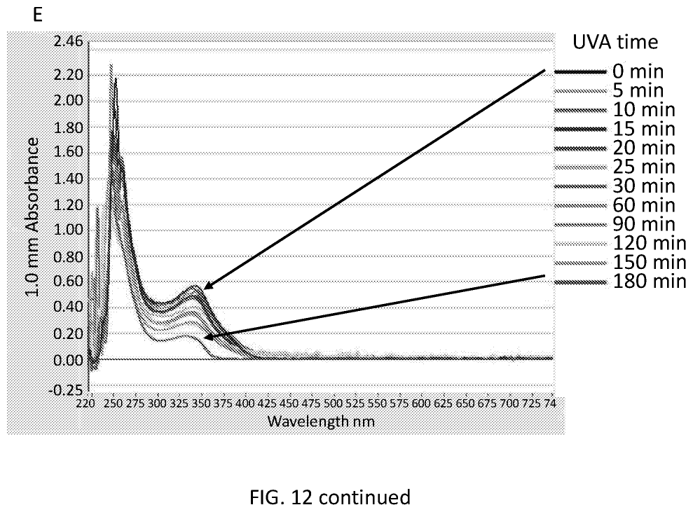

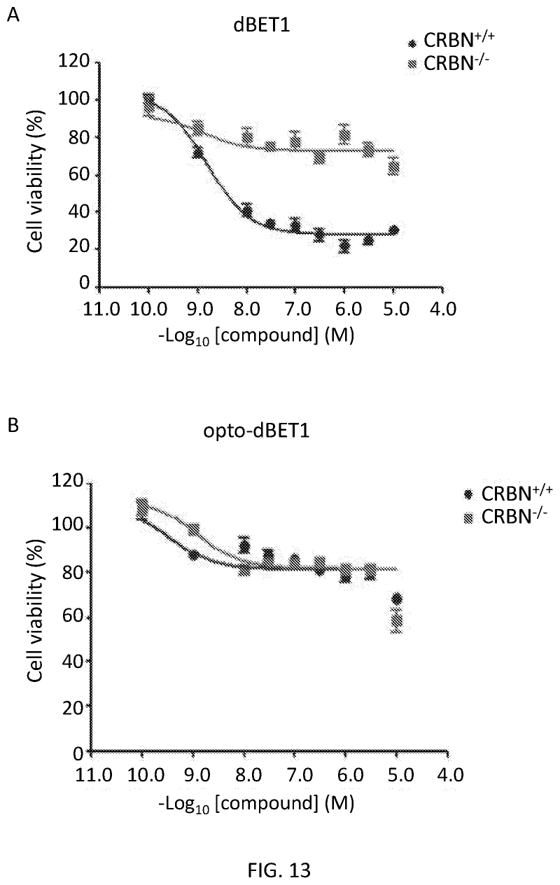

Opto-pomalidomide was shown to function in PROTACs as well. Opto-dBET1 was synthesized as shown in FIG. 8A and operated to promote degradation of bromodomain and extra terminal domain family proteins as shown in FIG. 8B. Like opto- pomalidomide, opto-dBET1 (FIG. 9A) was shown to be able to be efficiently uncaged by UVA irradiation in vitro (FIGS. 9B and 10A-C, 11A-B, and 12A-E).

dBET1 was shown to function as a molecular linker between a protein targeting moiety that recruits bromodomain family members (BRD2/3) (JQ1 derived) and CRBN-mediated ubiquitination (FIGS. 9C-D). Opto-dBET1 lost such function due to the blocking of its CRBN binding ability, thereby becoming incapable of guiding the ubiquitination of BRD2 and BRD3 (FIG. 9C-D). However, upon UVA irradiation leading to the uncaging process (FIG. 9B), opto-dBET1 regained its ability to promote ubiquitination of BRDs in cells (FIG. 9C-D). Furthermore, dBET1 degraded BRD 3/4 in a CRBN-dependent manner as well (FIG. 9E), while opto-dBET1 was largely inert and incapable of degrading BRD3/4 in this experimental condition (FIG. 9F).

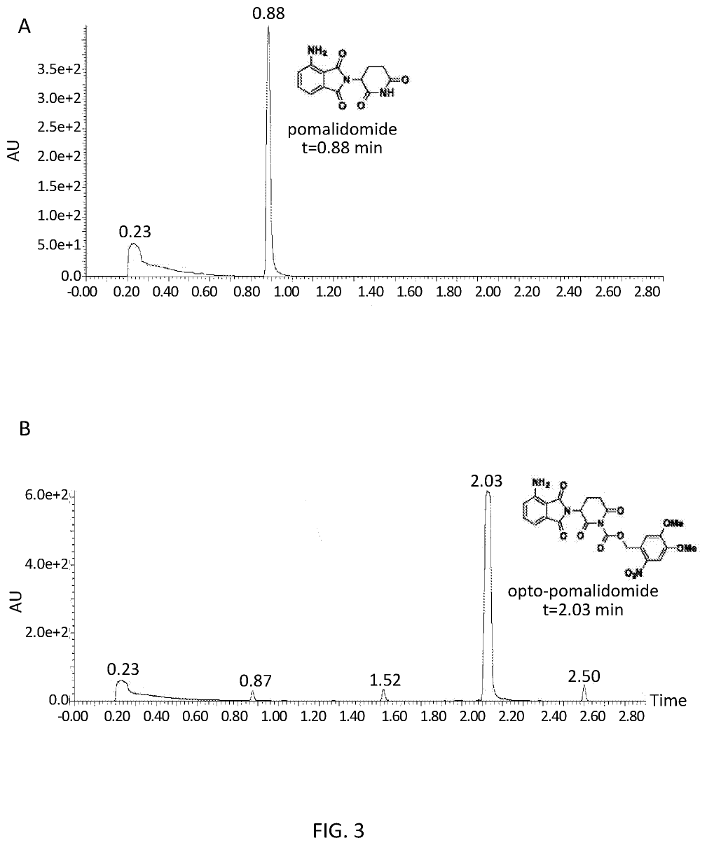

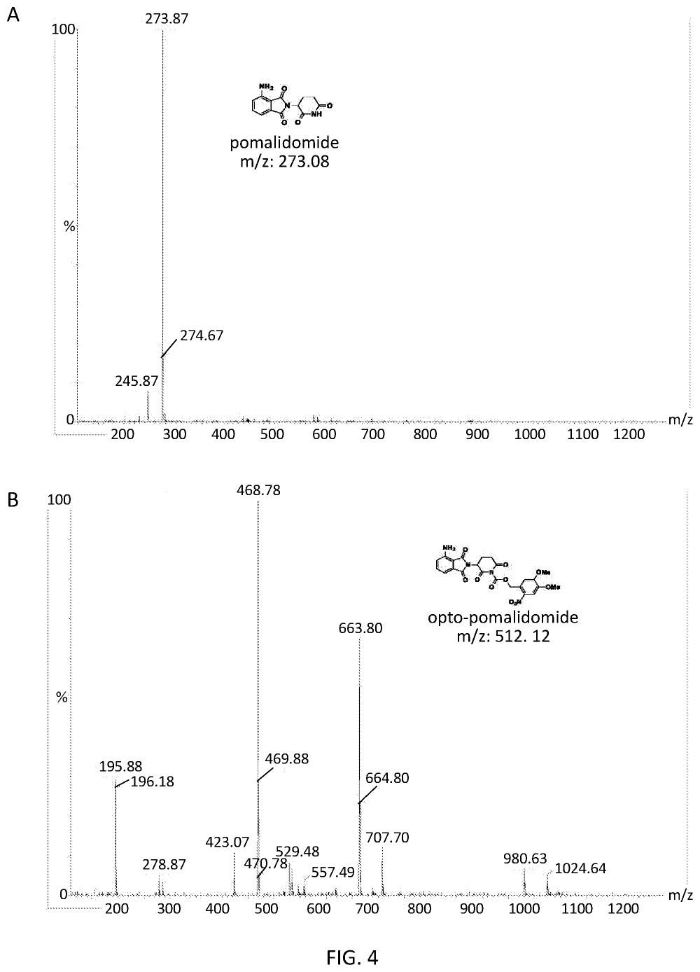

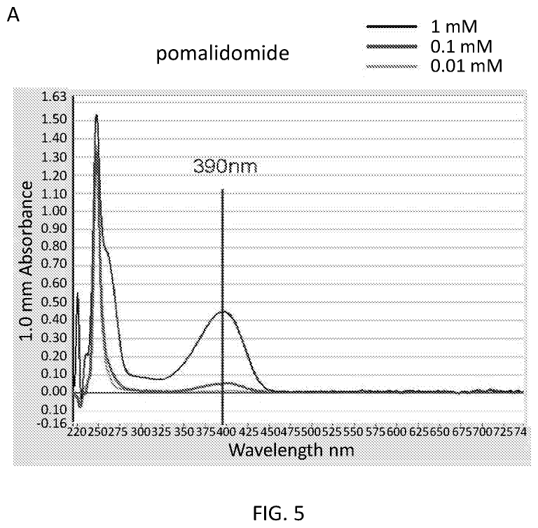

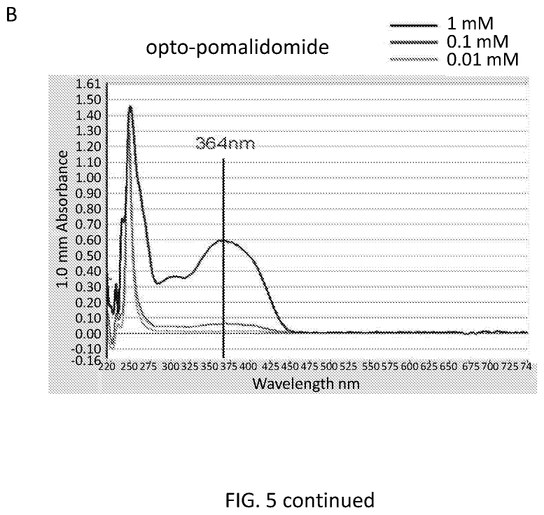

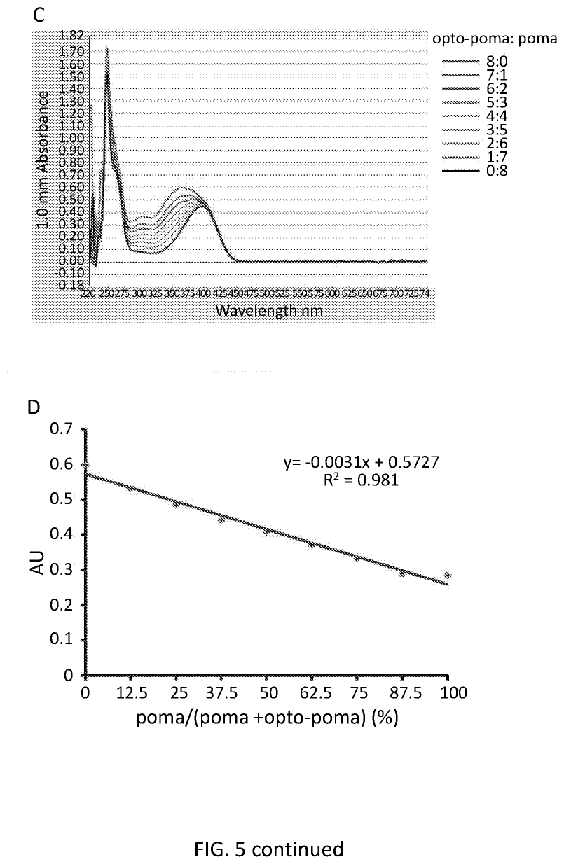

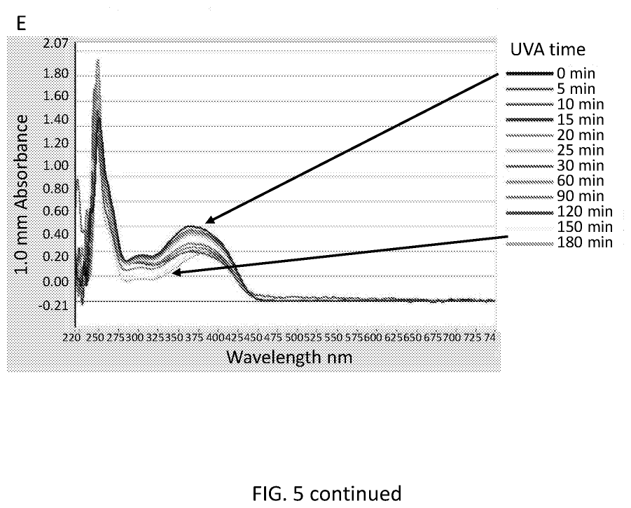

HEK293FT cells were pretreated with opto-dBET1 and then subjected to UVA irradiation (FIG. 9A-9K). Opto-dBET1, was activated in 5-15 minutes of UVA irradiation in cells, leading to the degradation of BRD3/4 (FIG. 3G) in a CRBN (FIG. 3H) and ubiqutin proteosome system (UPS)-dependent manner (FIG. 3I).

High dose of dBET1 has been reported to be lethal for cells due to its effects on completely depleting bromodomain family members that play critical roles in modulating enhancer and transcription activity of many genes. This shortcoming limits the further application of dBET1 in the clinic. As a potential solution to this emerging concern with regard to dBET1 and possibly other PROTACs in general, these results demonstrate that opto-dBET1 was an inert drug in inducing BRD degradation. With opto-PROTACs, this process can be specifically activated by light to achieve precise degradation in a temporal and spatial manner (FIGS. 9C-I). In keeping with this notion, dBET1 inhibited cell proliferation in a dose-dependent manner (FIG. 3J-K), while in the experimental conditions opto-dBET1 was relatively less toxic and could be activated by UVA irradiation to suppress cell proliferation (FIGS. 9J-K, 13A-B). Additionally, since the opto-PROTAC is inert until the decaging process, the amount decaged can be controlled by irradiation parameters such as power density, wavelength, spot size, and illumination frequency.

### Example 5

### Opto-PROTAC Analysis-Opto-dALK

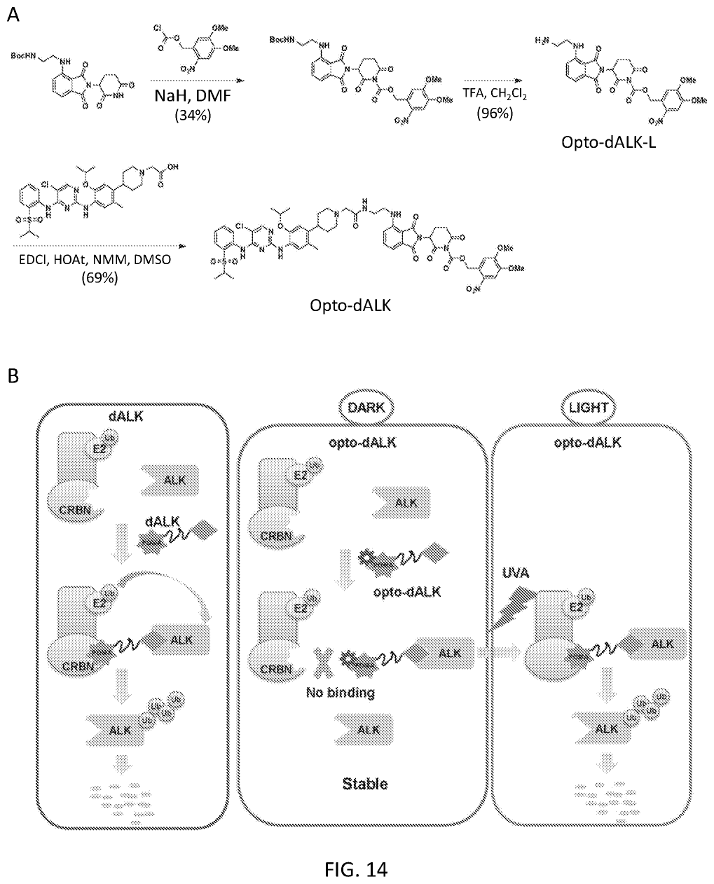

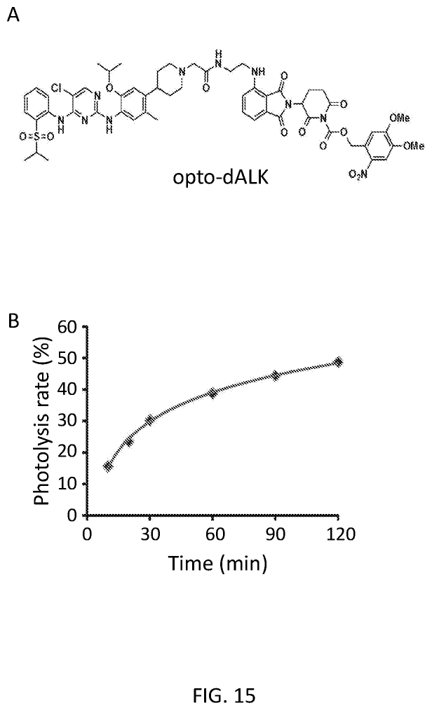

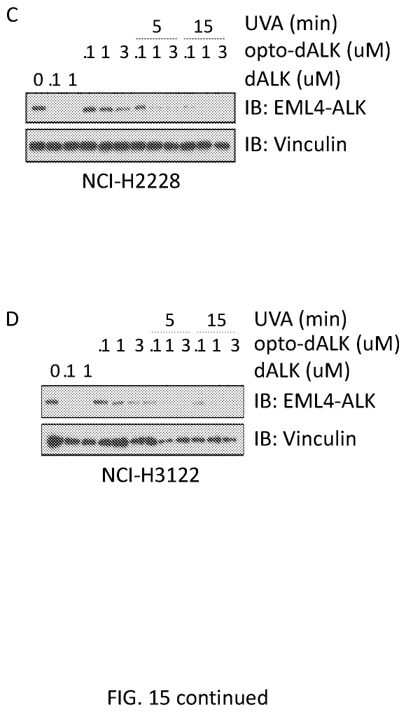

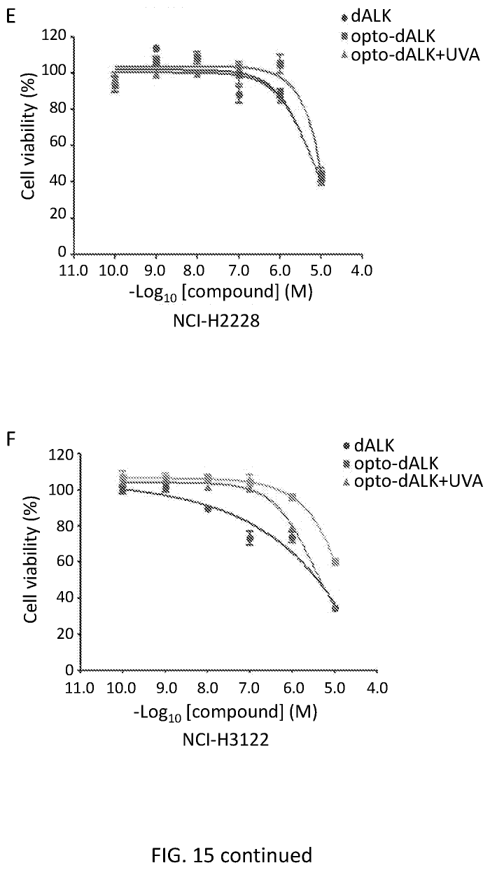

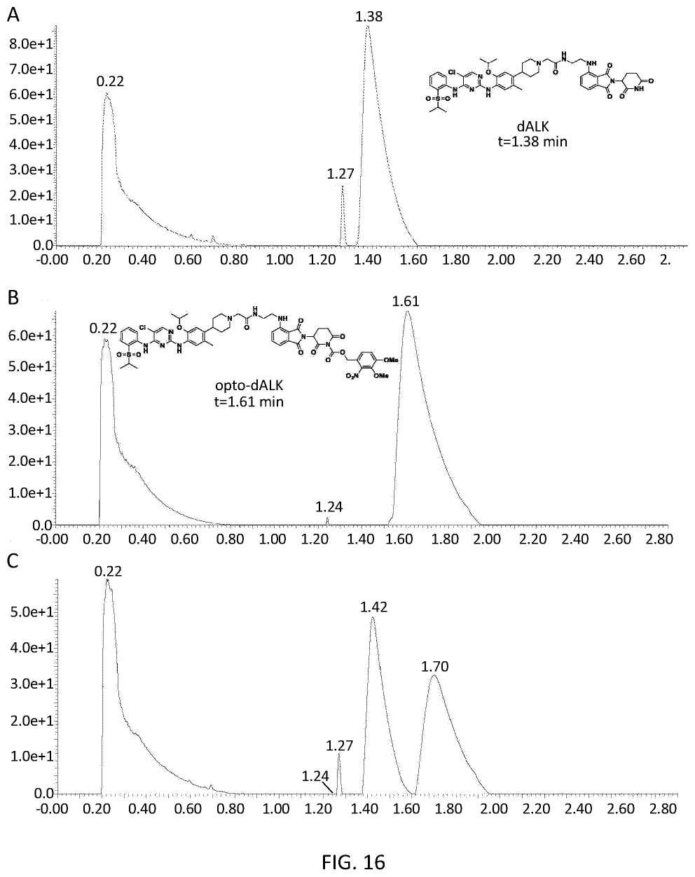

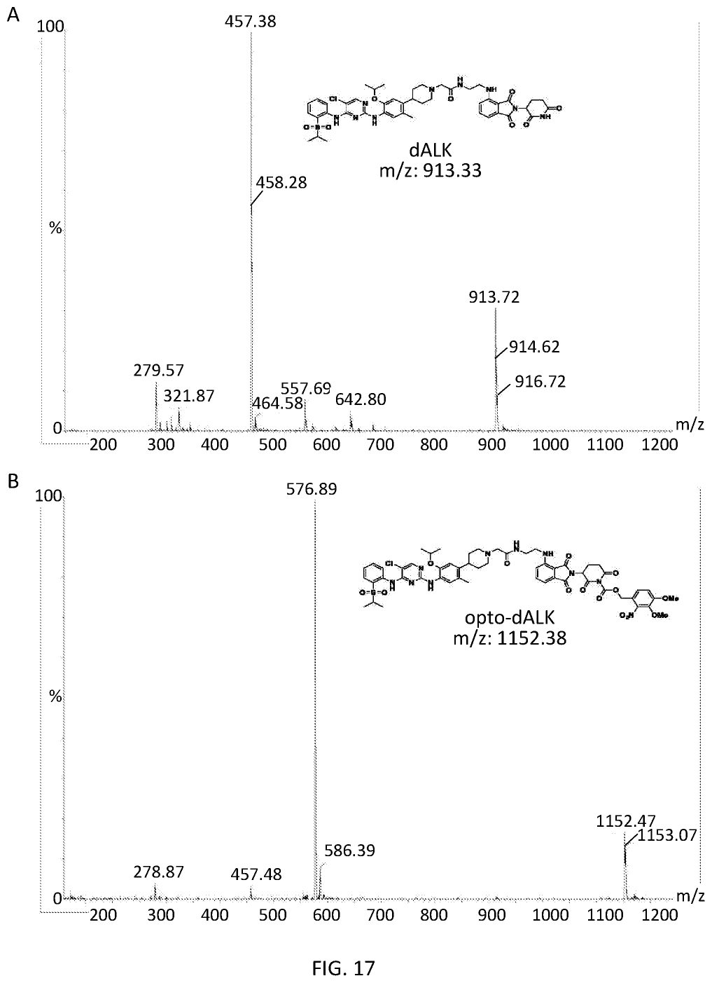

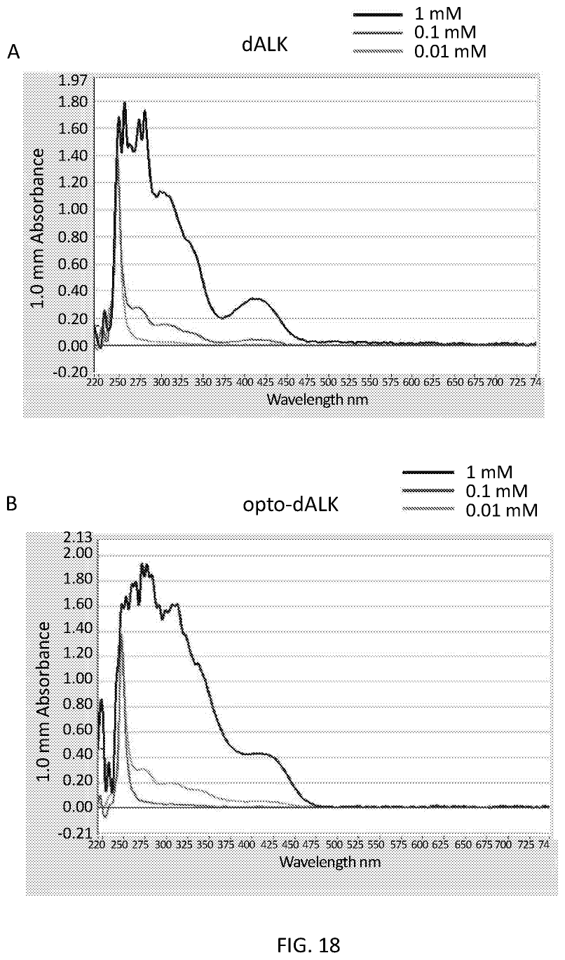

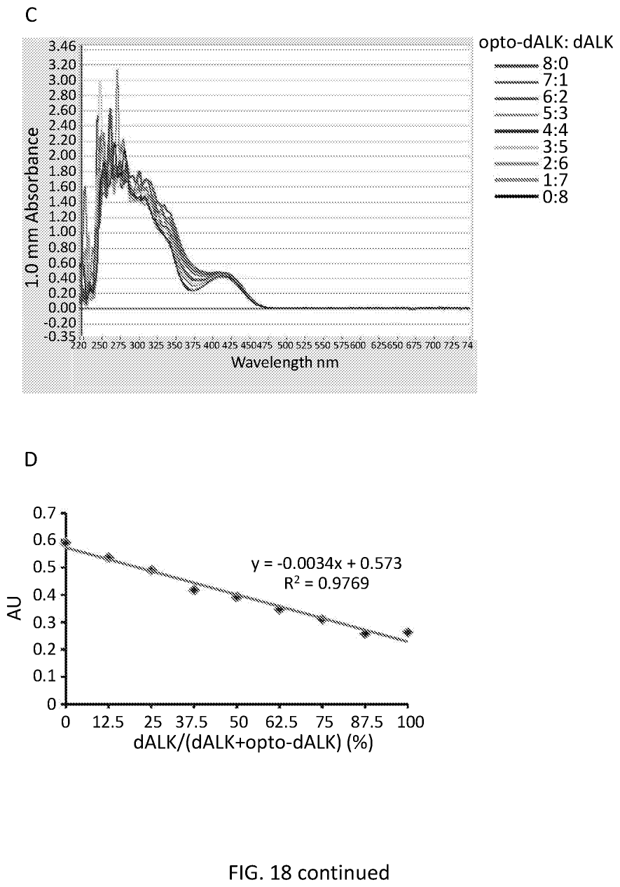

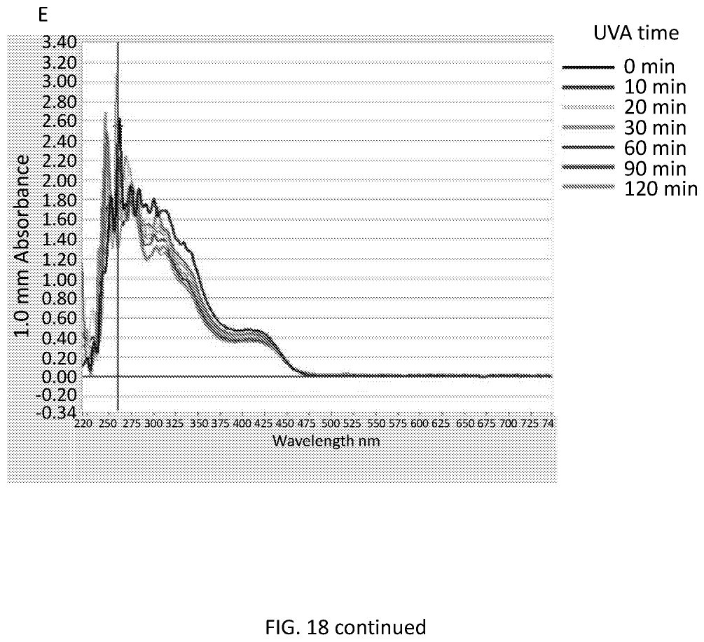

Opto-dALK was synthesized as shown in FIG. 14A and operated to promote degradation of bromodomain and extra terminal domain family proteins as shown in FIG. 14B. Like opto-pomamalide, and opto-dBET1, opto-dALK (FIG. 15A) was shown to be efficiently uncaged by UVA irradiation in vitro (FIGS. 15B, 16A-C, 17A-B, and 18A-E).

dALK promoted the degradation of EML4-ALK in two NSCLC cell lines, NCI-2228 and NCI-3122 in dose-dependent manner (FIGS. 19A-B). On the other hand, opto-ALK was largely inactive at basal level for guiding ALK degradation, but could be activated by UVA irradiation in drug-dose and UVA-dose dependent manner (FIGS. 15C-F). As a consequence, dALK inhibited NSCLC cell proliferation in a dose-dependent manner, while the ability of opto-dALK to block cell proliferation required prior uncaging by UVA irradiation (FIG. 15E-F, 19C-D).

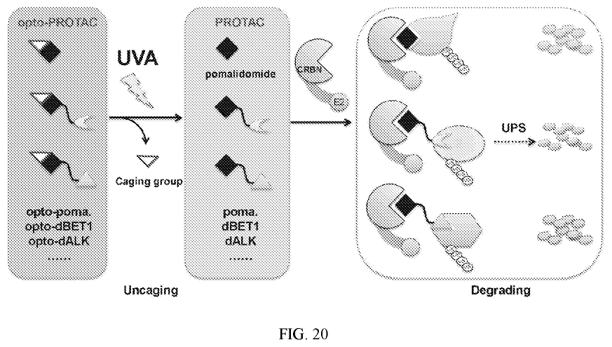

Taken together, these data provide experimental evidence for the development of light-control PROTACs, and enables PROTAC to be a precision medicine approach. Without wishing to be bound by theory, a schematic for an exemplary mechanism is found in FIG. 20.

### Specific Embodiments

Specific enumerated embodiments within the disclosure are described below.

Specific Embodiment 1. A compound having the structure of formula (I):

PB-L-ULB—PLG (I)
wherein ULB is a ubiquitin ligase binding moiety;
L is a linker;
PB is a protein binding moiety; and
PLG is an nitrophenyl based photolabile group (e.g., nitrobenzyl, orthro-nitrobenzyl, nitroveratryloxycarbonyl such as 6-nitroveratryloxycarbonyl, etc.),
wherein PLG is covalently bonded to ULB through a carbamate linkage;
or pharmaceutically acceptable salts thereof.

Specific embodiment 2. The compound according to specific embodiment 1, wherein the nitrogen of said carbamate linkage is a hydrogen binding moiety in ULB when said photolabile group is not present.

Specific embodiment 3. The compound according to specific embodiment 1 or 2, wherein PLG has the structure of formula (II):

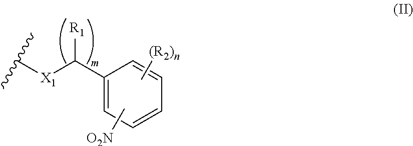

wherein

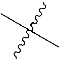

wherein indicates the point of attachment to the ULB group;
m is 0 (i.e., a bond), 1, or 2;
n is 0, 1, 2, 3, or 4;
X₁ is —O—, —C(O)—, —OC(O)—, —C(O)O—, —NR^{a}C(O)—, or —C(O)NR^{a}—;
R₁ is independently selected at each occurrence from hydrogen, alkyl (e.g., C₁-C₇ alkyl, C₁-C₃ alkyl, etc.), alkoxy (e.g., C₁-C₇ alkoxy, C₁-C₃ alkoxy, etc.);
R₂ is independently selected at each occurrence from hydrogen, —OC(O)R^{e}, —C(O)OR^{e}, —(C(R^{a})(R^{a}))₀₋₄—OC(O)N(R^{a})₂, halogen (e.g., F, Cl, Br, etc.), alkyl (e.g., C₁-C₇ alkyl, C₁-C₃ alkyl, etc.), and alkoxy (e.g., C₁-C₇ alkoxy, C₁-C₃ alkoxy, etc.), wherein two vicinal R₂ groups do not together form a ring;
R^{a} is independently selected at each occurrence from hydrogen, or alkyl (e.g., C₁-C₇ alkyl, C₁-C₃ alkyl, etc.); and
R^{e} is independently selected at each occurrence from hydrogen, or alkyl (e.g., C₁-C₇ alkyl, C₁-C₃ alkyl, etc.).

Specific embodiment 4. The compound according to specific embodiment 3, wherein said compound has the structure of formula (IIa):

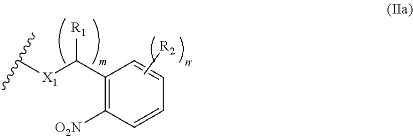

Specific embodiment 5. The compound according to specific embodiment 3 or 4, wherein n is 2 and at least one R₂ is alkoxy (e.g., C₁-C₃ alkoxy such as methoxy, etc.).

Specific embodiment 6. The compound according to any one of specific embodiments 3-5, wherein m is 1 and R₁ is hydrogen.

Specific embodiment 7. The compound according to any one of specific embodiments 3-6, wherein said PLG group has the structure of formula (IIb):

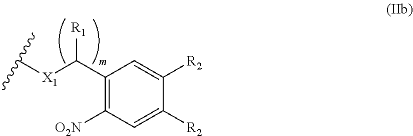

Specific embodiment 8. The compound according to specific embodiment 3, wherein said PLG group has the structure of formula (IIc):

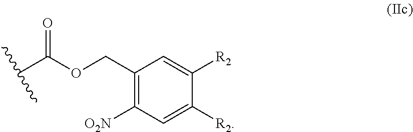

Specific embodiment 9. The compound according to specific embodiment 7 or 8, wherein each R₂ is methoxy.

Specific embodiment 10. The compound according to any one of specific embodiments 1-9, wherein said ULB binds to an E3 ubiquitin ligase.

Specific embodiment 11. The compound according to specific embodiment 10, wherein the E3 ubiquitin ligase comprises von Hippel Lindau (VHL) E3 ubiquitin ligase, β-Transducin Repeat Containing (β-TRCP) E3 Ubiquitin Protein Ligase, Mouse Double Minute 2 (Mdm2) E3 Ubiquitin Protein Ligase, or a Cereblon (CRBN) E3 Ubiquitin ligase.

Specific embodiment 12. The compound according to any one of specific embodiments 1-11, wherein said compound has the structure of formula (III):

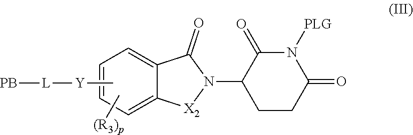

wherein p is 0 (i.e., each R3 is hydrogen), 1, 2, or, 3;
R₃ is independently selected at each occurrence from hydrogen, —N(R^{a})(R^{a}), alkyl (e.g., C₁-C₇ alkyl, C₁-C₃ alkyl, etc.), or alkoxy (e.g., C₁-C₇ alkoxy, C₁-C₃ alkoxy, etc.);
X₂ is C(O), CH, CR^{a}, or NR^{a};
Y is absent (i.e, a bond), —O—, —C(O)—, —OC(O)—, —C(O)O—, —NR^{a}C(O)—, or —C(O)NR^{a}—;
R^{a} is independently selected at each occurrence from hydrogen, or alkyl (e.g., C₁-C₇ alkyl, C₁-C₃ alkyl, etc.).

Specific embodiment 13. The compound according to any one of specific embodiments 1-12, wherein ULB is lenalidomide derived, pomalidomide derived, or thalidomide derived.

Specific embodiment 14. The compound according to specific embodiment 13, wherein said ULB is lenalidomide derived and said compound has the structure of formula (IIIa) or (IIIb):

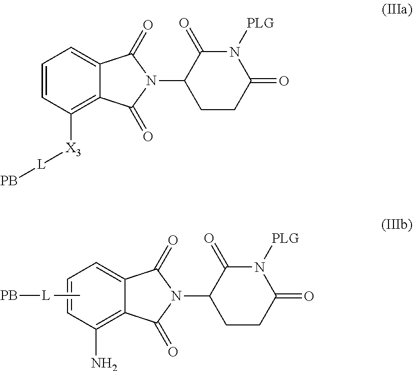

wherein X₃ is —NH— or —O—.

Specific embodiment 15. The compound according to specific embodiment 13, wherein said ULB is thalidomide derived and said compound has the structure of formula (IIIc):

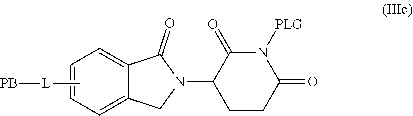

Specific embodiment 16. The compound according to specific embodiment 13, wherein said ULB is pomalidomide derived and said compound has the structure of formula (IIId) or (IIIe):

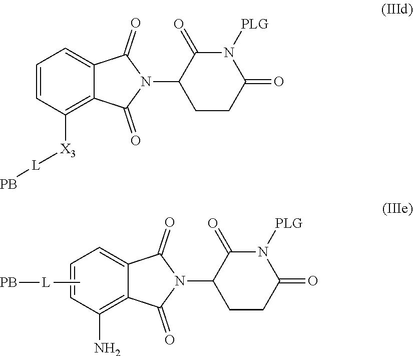

wherein X₃ is —NH— or —O—.

Specific embodiment 17. The compound according to any one of specific embodiments 1-16, wherein said compound has the structure of formula (IV):

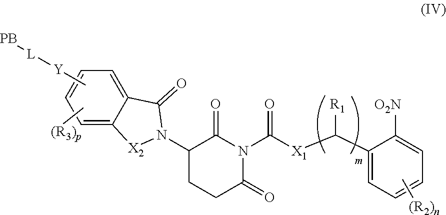

wherein m is 0, 1, or 2;
n is 0, 1, 2, 3, or 4;
p is 0, 1, 2, or 3;
R₁ is independently selected at each occurrence from hydrogen, alkyl (e.g., C₁-C₇ alkyl, C₁-C₃ alkyl, etc.), alkoxy (e.g., C₁-C₇ alkoxy, C₁-C₃ alkoxy, etc.);
R₂ is independently selected at each occurrence from hydrogen, —OC(O)R^{e}, —C(O)OR^{e}, alkyl (e.g., C₁-C₇ alkyl, C₁-C₃ alkyl, etc.), and alkoxy (e.g., C₁-C₇ alkoxy, C₁-C₃ alkoxy, etc.), wherein two vicinal R₂ groups do not together form a ring;
R₃ is independently selected at each occurrence from hydrogen, —N(R^{a})(R^{a}), alkyl (e.g., C₁-C₇ alkyl, C₁-C₃ alkyl, etc.), or alkoxy (e.g., C₁-C₇ alkoxy, C₁-C₃ alkoxy, etc.);
X₁ is —O—, —C(O)—, —NR^{a}—, —OC(O)—, —C(O)O—, —NR^{a}C(O)—, or —C(O)NR^{a}—;
X₂ is C(O), CH₂, C(R^{a})(R^{a}), or NR^{a};
Y is absent (i.e., a bond), —O—, —C(O)—, —OC(O)—, —C(O)O—, —NR^{a}C(O)—, or —C(O)NR^{a}—;
R^{a} is independently selected at each occurrence from hydrogen, or alkyl (e.g., C₁-C₇ alkyl, C₁-C₃ alkyl, etc.); and
R^{e} is independently selected at each occurrence from hydrogen, or alkyl (e.g., C₁-C₇ alkyl, C₁-C₃ alkyl, etc.).

Specific embodiment 18. The compound according to any one of specific embodiments 1-17, wherein said protein binding moiety is a protein inhibitor.

Specific embodiment 19. The compound according to any one of specific embodiments 1-17, wherein said protein binding moiety is a tyrosine kinase inhibitor, a BRAF-mutant inhibitor, or a MEK inhibitor.

Specific embodiment 20. The compound according to any one of specific embodiments 1-19, wherein said protein binding moiety binds to one or more of Abelson Murine Leukemia (ABL) Proteins (e.g., c-ABL, etc.), Breakpoint Cluster Region Protein (BCR), BCR-ABL fusion proteins, Bromodomain and Extra Terminal Domain (BRD) Family proteins (e.g., BRD2, BRD3, BRDT, BRD4, etc.), anaplastic lymphoma kinase (ALK) protein, and echinoderm microtubule-associated protein like (EML)-ALK fusion proteins.

Specific embodiment 21. The compound according to any one of specific embodiments 1-20, wherein PB has an affinity for its target protein (Ka) of less than 1 mM (e.g. from 500 μM to 1 mM, etc.) or less than 500 μM (e.g., less than 450 μM, less than 400 μM, less than 350 μM, less than 300 μM, less than 250 μM, less than 200 μM, less than 150 μM, less than 100 μM, less than 100 μM, less than 50 μM, less than 10 μM, less than 1 μM, less than 500 nm, less than 100 nM, less than 50 nM, less than 10 nM, less than 1 nM, etc.).

Specific embodiment 22. The compound according to any one of specific embodiments 1-21, wherein the protein binding moiety is derived from crizotinib, certinib, alectinib, brigatinib, or JQ1.

Specific embodiment 23. The compound according to any one of specific embodiments 1-22, wherein PB is:

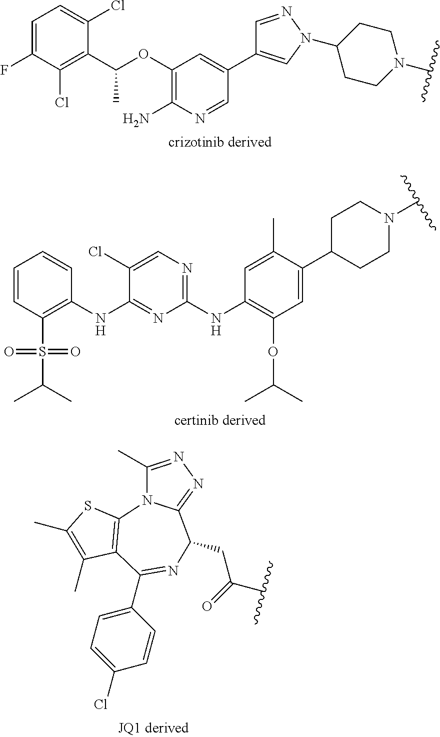 [SMILES: *N1CCC(n2cc(-c3cnc(N)c(O[C@H](C)c4c(Cl)ccc(F)c4Cl)c3)cn2)CC1 ; *N1CCC(c2cc(OC(C)C)c(Nc3ncc(Cl)c(Nc4ccccc4S(=O)(=O)C(C)C)n3)cc2C)CC1 ; *C(=O)C[C@@H]1N=C(c2ccc(Cl)cc2)c2c(sc(C)c2C)-n2c(C)nnc21]

wherein

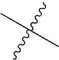

indicates the point of attachment to the L group.

Specific embodiment 24. The compound according to any one of specific embodiments 1-23, wherein said compound has the structure:

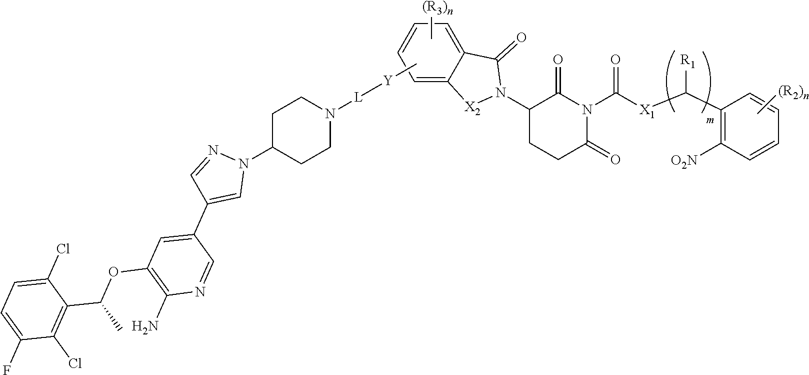

wherein m is 0, 1, or 2;
n is 0, 1, 2, 3, or 4;
p is 1, 2, or, 3;
R₁ is independently selected at each occurrence from hydrogen, alkyl, and alkoxy;
R₂ is independently selected at each occurrence from hydrogen, —OC(O)R^{e}, —C(O)OR^{e}, alkyl, and alkoxy, wherein two vicinal R₂ groups do not together form a ring;
R₃ is independently selected at each occurrence from hydrogen, alkyl, or alkoxy;
X₁ is —O—, —C(O)—, —OC(O)—, —C(O)O—, —NR^{a}C(O)—, or —C(O)NR^{a}—;
X₂ is C(O), CH, CR^{a}, or NR^{a};
Y is a bond, —O—, —C(O)—, —OC(O)—, —C(O)O—, —NR^{a}C(O)—, or —C(O)NR^{a}—;
R^{a} is independently selected at each occurrence from hydrogen, and alkyl; and
R^{e} is independently selected at each occurrence from hydrogen, and alkyl.

Specific embodiment 25. The compound according to any one of specific embodiments 1-23, wherein said compound has the structure:

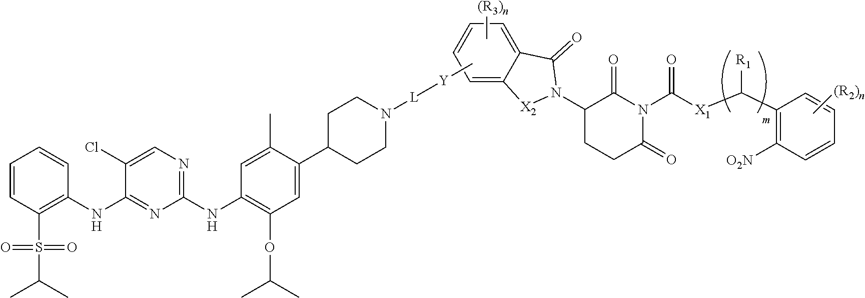

wherein m is 0, 1, or 2;
n is 0, 1, 2, 3, or 4;
p is 1, 2, or, 3;
R₁ is independently selected at each occurrence from hydrogen, alkyl (e.g., C₁-C₇ alkyl, C₁-C₃ alkyl, etc.), alkoxy (e.g., C₁-C₇ alkoxy, C₁-C₃ alkoxy, etc.);
R₂ is independently selected at each occurrence from hydrogen, —OC(O)R^{e}, —C(O)OR^{e}, alkyl (e.g., C₁-C₇ alkyl, C₁-C₃ alkyl, etc.), and alkoxy (e.g., C₁-C₇ alkoxy, C₁-C₃ alkoxy, etc.), wherein two vicinal R₂ groups do not together form a ring;
R₃ is independently selected at each occurrence from hydrogen, alkyl (e.g., C₁-C₇ alkyl, C₁-C₃ alkyl, etc.), or alkoxy (e.g., C₁-C₇ alkoxy, C₁-C₃ alkoxy, etc.);
X₁ is —O—, —C(O)—, —OC(O)—, —C(O)O—, —NR^{a}C(O)—, or —C(O)NR^{a}—;
X₂ is C(O), CH, CR^{a}, or NR^{a};
Y is a bond, —O—, —C(O)—, —OC(O)—, —C(O)O—, —NR^{a}C(O)—, or —C(O)NR^{a}—;
R^{a} is independently selected at each occurrence from hydrogen, or alkyl (e.g., C₁-C₇ alkyl, C₁-C₃ alkyl, etc.); and
R^{e} is independently selected at each occurrence from hydrogen, or alkyl (e.g., C₁-C₇ alkyl, C₁-C₃ alkyl, etc.).

Specific embodiment 26. The compound according to any one of specific embodiments 1-23, wherein said compound has the structure:

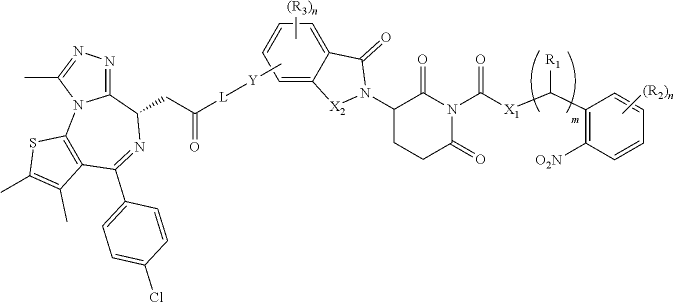

wherein m is 0, 1, or 2;
n is 0, 1, 2, 3, or 4;
p is 1, 2, or, 3;
R₁ is independently selected at each occurrence from hydrogen, alkyl (e.g., C₁-C₇ alkyl, C₁-C₃ alkyl, etc.), alkoxy (e.g., C₁-C₇ alkoxy, C₁-C₃ alkoxy, etc.);
R₂ is independently selected at each occurrence from hydrogen, —OC(O)R^{e}, —C(O)OR^{e}, alkyl (e.g., C₁-C₇ alkyl, C₁-C₃ alkyl, etc.), and alkoxy (e.g., C₁-C₇ alkoxy, C₁-C₃ alkoxy, etc.), wherein two vicinal R₂ groups do not together form a ring;
R₃ is independently selected at each occurrence from hydrogen, alkyl (e.g., C₁-C₇ alkyl, C₁-C₃ alkyl, etc.), or alkoxy (e.g., C₁-C₇ alkoxy, C₁-C₃ alkoxy, etc.);
X₁ is —O—, —C(O)—, —OC(O)—, —C(O)O—, —NR^{a}C(O)—, or —C(O)NR^{a}—;
X₂ is C(O), CH, CR^{a}, or NR^{a};
Y is a bond, —O—, —C(O)—, —OC(O)—, —C(O)O—, —NR^{a}C(O)—, or —C(O)NR^{a}—;
R^{a} is independently selected at each occurrence from hydrogen or alkyl (e.g., C₁-C₇ alkyl, C₁-C₃ alkyl, etc.); and
R^{e} is independently selected at each occurrence from hydrogen or alkyl (e.g., C₁-C₇ alkyl, C₁-C₃ alkyl, etc.).

Specific embodiment 27. The compound according any one of specific embodiments 1-26, wherein L is a divalent hydrocarbon selected from saturated or unsaturated alkylene (e.g., branched alkylelene, linear alkylene, cycloalkylene, C₁-C₂₂ branched alkylelene, C₁-C₂₂ linear alkylene, C₃-C₂₂ cycloalkylene, C₁-C₁₀ branched alkylelene, C₁-C₁₀ linear alkylene, C₃-C₁₀ cycloalkylene, C₁-C₈ branched alkylelene, C₁-C₈ linear alkylene, C₃-C₈ cycloalkylene, etc.), C₁-C₂₂ saturated or unsaturated heteroalkylene (e.g., branched heteroalkylelene, linear heteroalkylene, heterocycloalkylene, C₁-C₂₂ branched heteroalkylelene, C₁-C₂₂ linear heteroalkylene, C₃-C₂₂ heterocycloalkylene, C₁-C₁₀ branched heteroalkylelene, C₁-C₁₀ linear heteroalkylene, C₃-C₁₀ heterocycloalkylene, C₁-C₈ branched heteroalkylelene, C₁-C₈ linear heteroalkylene, C₃-C₈heterocycloalkylene, etc.), arylene (e.g., C₅-C₂₂ arylene, etc.), heteroarylene (e.g., C₅-C₂₂ heteroarylene, etc.), or combinations thereof

Specific embodiment 28. The compound according to any one of specific embodiments 1-27, wherein L comprises one or more of —(C(R^{a})(R^{a}))₁₋₈—, —(OC(R^{a})(R^{a}))₁₋₈—, —(OC(R^{a})(R^{a})—C(R^{a})(R^{a}))₁₋₈—, —N(R^{a})—, —O—, or —C(O)—;
wherein R^{a} is independently selected at each occurrence from hydrogen or alkyl (e.g., C₁-C₇ alkyl, C₁-C₄ alkyl, etc.).

Specific embodiment 29. The compound according to any one of specific embodiments 1-28, wherein L is —(CH₂)₀₋₈—C(O)NH—(CH₂)₀₋₈—, —C(O)NH—(CH₂)₀₋₈—, —NHC(O)—(CH₂)₀₋₈—, —NH—(CH₂)₀₋₈—, or —C(O)—(CH₂)₀₋₈—, or combinations thereof.

Specific embodiment 30. The compound according to specific embodiment 29, wherein said compound has the structure of formula (Va) or (Vb):

PB—NH—(CH₂)₁₋₈—NH—C(O)—ULB—PLG (Va)

PB—(CH₂)₁₋₈—NH—C(O)—(CH₂)₁₋₈—ULB—PLG (Yb)

Specific embodiment 31. The compound according to any one of specific embodiments 1-30, wherein said compound has the structure:

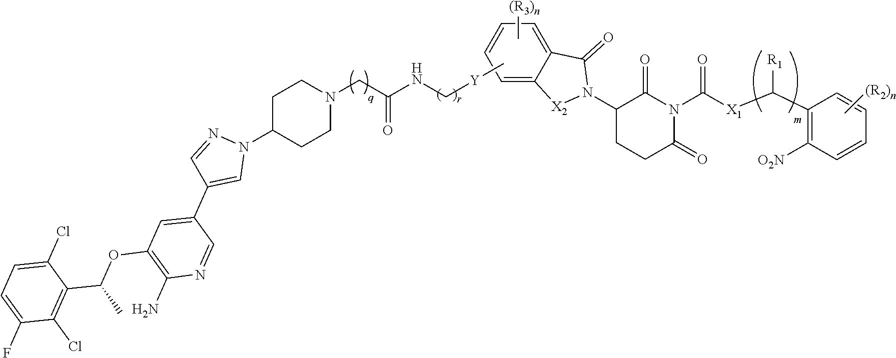

wherein m is 0, 1, or 2;
n is 0, 1, 2, 3, or 4;
p is 1, 2, or, 3;
q and r and independently 0, 1, 2, 3, 4, 5, 6, 7, or 8;
R₁ is independently selected at each occurrence from hydrogen, alkyl (e.g., C₁-C₇ alkyl, C₁-C₃ alkyl, etc.), alkoxy (e.g., C₁-C₇ alkoxy, C₁-C₃ alkoxy, etc.);
R₂ is independently selected at each occurrence from hydrogen, —OC(O)R^{e}, —C(O)OR^{e}, alkyl (e.g., C₁-C₇ alkyl, C₁-C₃ alkyl, etc.), and alkoxy (e.g., C₁-C₇ alkoxy, C₁-C₃ alkoxy, etc.), wherein two vicinal R₂ groups do not together form a ring;
R₃ is independently selected at each occurrence from hydrogen, alkyl (e.g., C₁-C₇ alkyl, C₁-C₃ alkyl, etc.), or alkoxy (e.g., C₁-C₇ alkoxy, C₁-C₃ alkoxy, etc.);
X₂ is C(O), CH, CR^{a}, or NR^{a};
Y is a bond, —O—, —C(O)—, —OC(O)—, —C(O)O—, —NR^{a}C(O)—, or —C(O)NR^{a}—;
R^{a} is independently selected at each occurrence from hydrogen, or alkyl (e.g., C₁-C₇ alkyl, C₁-C₃ alkyl, etc.); and
R^{e} is independently selected at each occurrence from hydrogen, or alkyl (e.g., C₁-C₇ alkyl, C₁-C₃ alkyl, etc.).

Specific embodiment 32. The compound according to any one of specific embodiments 1-30, wherein said compound has the structure:

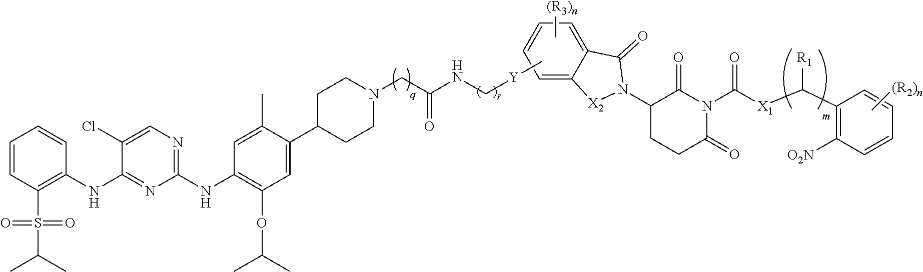

wherein m is 0, 1, or 2;
n is 0, 1, 2, 3, or 4;
p is 1, 2, or, 3;
q and r and independently 0, 1, 2, 3, 4, 5, 6, 7, or 8;
R₁ is independently selected at each occurrence from hydrogen, alkyl (e.g., C₁-C₇ alkyl, C₁-C₃ alkyl, etc.), alkoxy (e.g., C₁-C₇ alkoxy, C₁-C₃ alkoxy, etc.);
R₂ is independently selected at each occurrence from hydrogen, —OC(O)R^{e}, —C(O)OR^{e}, alkyl (e.g., C₁-C₇ alkyl, C₁-C₃ alkyl, etc.), and alkoxy (e.g., C₁-C₇ alkoxy, C₁-C₃ alkoxy, etc.), wherein two vicinal R₂ groups do not together form a ring;
R₃ is independently selected at each occurrence from hydrogen, alkyl (e.g., C₁-C₇ alkyl, C₁-C₃ alkyl, etc.), or alkoxy (e.g., C₁-C₇ alkoxy, C₁-C₃ alkoxy, etc.);
X₂ is C(O), CH, CR^{a}, or NR^{a};
Y is a bond, —O—, —C(O)—, —OC(O)—, —C(O)O—, —NR^{a}C(O)—, or —C(O)NR^{a}—;
R^{a} is independently selected at each occurrence from hydrogen, or alkyl (e.g., C₁-C₇ alkyl, C₁-C₃ alkyl, etc.); and
R^{e} is independently selected at each occurrence from hydrogen, or alkyl (e.g., C₁-C₇ alkyl, C₁-C₃ alkyl, etc.).

Specific embodiment 33. The compound according to any one of specific embodiments 1-30, wherein said compound has the structure:

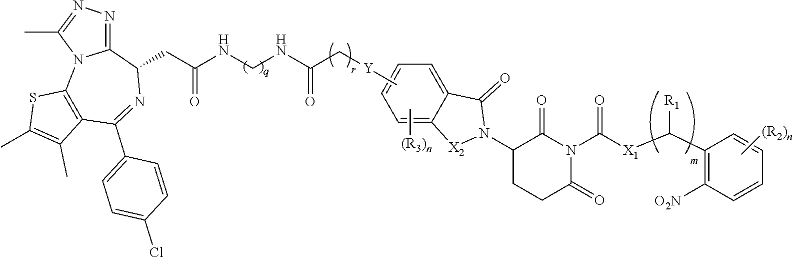

wherein m is 0, 1, or 2;
n is 0, 1, 2, 3, or 4;
p is 1, 2, or, 3;
R₁ is independently selected at each occurrence from hydrogen, alkyl (e.g., C₁-C₇ alkyl, C₁-C₃ alkyl, etc.), alkoxy (e.g., C₁-C₇ alkoxy, C₁-C₃ alkoxy, etc.);
R₂ is independently selected at each occurrence from hydrogen, —OC(O)R^{e}, —C(O)OR^{e}, alkyl (e.g., C₁-C₇ alkyl, C₁-C₃ alkyl, etc.), and alkoxy (e.g., C₁-C₇ alkoxy, C₁-C₃ alkoxy, etc.), wherein two vicinal R₂ groups do not together form a ring;
R₃ is independently selected at each occurrence from hydrogen, alkyl (e.g., C₁-C₇ alkyl, C₁-C₃ alkyl, etc.), or alkoxy (e.g., C₁-C₇ alkoxy, C₁-C₃ alkoxy, etc.);
X₂ is C(O), CH, CR^{a}, or NR^{a};
Y is a bond, —O—, —C(O)—, —OC(O)—, —C(O)O—, —NR^{a}C(O)—, or —C(O)NR^{a}—;
R^{a} is independently selected at each occurrence from hydrogen or alkyl (e.g., C₁-C₇ alkyl, C₁-C₃ alkyl, etc.); and
R^{e} is independently selected at each occurrence from hydrogen or alkyl (e.g., C₁-C₇ alkyl, C₁-C₃ alkyl, etc.).

Specific embodiment 34. A compound having the structure:

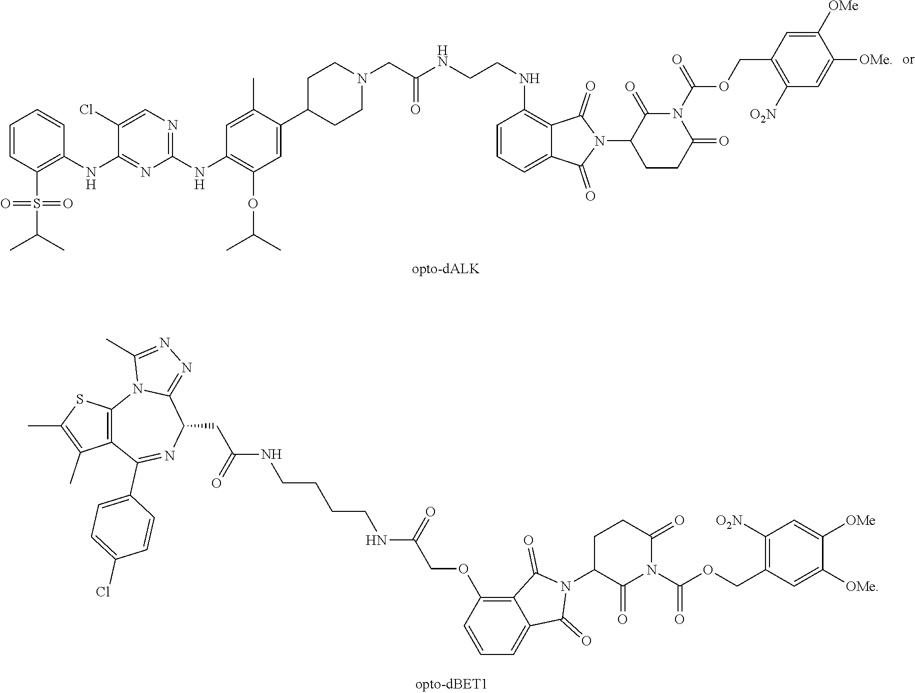 [SMILES: COc1cc(COC(=O)N2C(=O)CCC(N3C(=O)c4cccc(NCCNC(=O)CN5CCC(c6cc(OC(C)C)c(Nc7ncc(Cl)c(Nc8ccccc8S(=O)(=O)C(C)C)n7)cc6C)CC5)c4C3=O)C2=O)c([N+](=O)[O-])cc1OC ; COc1cc(COC(=O)N2C(=O)CCC(N3C(=O)c4cccc(OCC(=O)NCCCCNC(=O)C[C@@H]5N=C(c6ccc(Cl)cc6)c6c(sc(C)c6C)-n6c(C)nnc65)c4C3=O)C2=O)c([N+](=O)[O-])cc1OC]

Specific embodiment 35. A compound having the structure of formula (VI):

Specific embodiment 36. The compound according to any one of specific embodiments 1-16, wherein said compound has the structure of formula (VI):

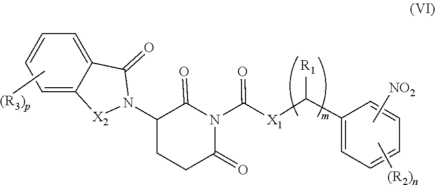

wherein m is 0, 1, or 2;
n is 0, 1, 2, 3, or 4;
p is 0, 1, 2, 3, or 4;
R₁ is independently selected at each occurrence from hydrogen, alkyl (e.g., C₁-C₇ alkyl, C₁-C₃ alkyl, etc.), alkoxy (e.g., C₁-C₇ alkoxy, C₁-C₃ alkoxy, etc.);
R₂ is independently selected at each occurrence from hydrogen, —OC(O)R^{e}, —C(O)OR^{e}, alkyl (e.g., C₁-C₇ alkyl, C₁-C₃ alkyl, etc.), and alkoxy (e.g., C₁-C₇ alkoxy, C₁-C₃ alkoxy, etc.);
R₃ is independently selected at each occurrence from hydrogen, —N(R^{a})(R^{a}), alkyl (e.g., C₁-C₇ alkyl, C₁-C₃ alkyl, etc.), or alkoxy (e.g., C₁-C₇ alkoxy, C₁-C₃ alkoxy, etc.);
X₁ is —O—, —C(O)—, —NR^{a}—, —OC(O)—, —C(O)O—, —NR^{a}C(O)—, or —C(O)NR^{a}—;
X₂ is C(O), CH₂, C(R^{a})(R^{a}), or NR^{a};
R^{a} is independently selected at each occurrence from hydrogen, or alkyl (e.g., C₁-C₇ alkyl, C₁-C₃ alkyl, etc.); and
R^{e} is independently selected at each occurrence from hydrogen, or alkyl (e.g., C₁-C₇ alkyl, C₁-C₃ alkyl, etc.);
or pharmaceutically acceptable salts thereof.

Specific embodiment 37. The compound according to specific embodiment 36, wherein two vicinal R₂ groups do not together form a ring.

Specific embodiment 38. The compound according to any specific embodiment 36 or 37, wherein said compound has the structure of formula (VIa):

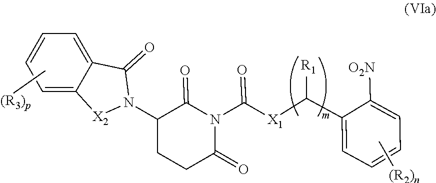

Specific embodiment 39. The compound according to any one of specific embodiments 36-38, wherein said compound has the structure of formula (VIb):

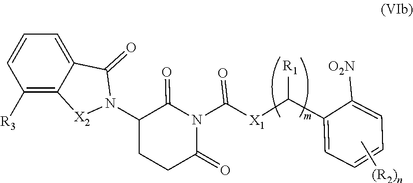

Specific embodiment 40. The compound according to any one of specific embodiments 36-39, wherein said compound has the structure of formula (VIc):

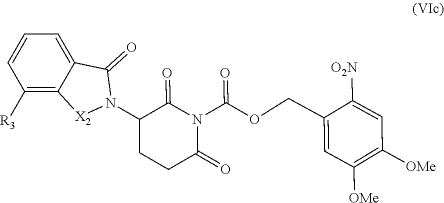

Specific embodiment 41. The compound according to any one of specific embodiments 36-40, wherein said compound is

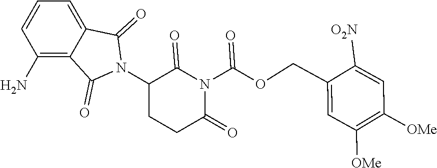 [SMILES: COc1cc(COC(=O)N2C(=O)CCC(N3C(=O)c4cccc(N)c4C3=O)C2=O)c([N+](=O)[O-])cc1OC]

Specific embodiment 42. A pharmaceutical composition comprising the compound according to any one of specific embodiments 1-41 and one or more pharmaceutically acceptable salts, carriers, or diluents.

Specific embodiment 43. The pharmaceutical composition according to specific embodiment 42, wherein said composition is formulated as a topical composition (e.g., ointment, gel, etc.).

Specific embodiment 44. The pharmaceutical composition according to specific embodiment 42 or 43, wherein said composition comprises from 0.1%-90% (e.g., 0.1%-50%, 0.1%-20%, 0.1%-10%, etc.) of said compound by weight of the composition.

Specific embodiment 45. A method for the treatment of a proliferative disease in a patient in need thereof comprising administration of the compound according to any one of specific embodiments 1-41 or the pharmaceutical composition according to any one of specific embodiments 42-44 to said patient.

Specific embodiment 46. The method according to specific embodiment 45, further comprising irradiating said patient with electromagnetic radiation comprising photons of one or more wavelengths and a power density for an irradiation time period sufficient to induce the separation of said photolabile group from the ubiquitin ligase binding moiety of said compound.

Specific embodiment 47. The method according to specific embodiment 46, wherein said photons have one or more wavelengths between 300 and 450 nm.

Specific embodiment 48. The method according to specific embodiment 47, wherein said electromagnetic radiation has a wavelength spectrum with a maximum at one or more wavelengths between 300 and 450 nm.

Specific embodiment 49. The method according to any one of specific embodiments 46-48, wherein said electromagnetic radiation has a wavelength spectrum with a maximum between 325-375 nm.

Specific embodiment 50. The method according to specific embodiment 46, wherein said proliferative disease is localized in a specific area of said patient,

wherein said compound is administered to one or more portions of said specific area, and said electromagnetic radiation is irradiated to one or more portions of said specific area.

Specific embodiment 51. The method according to specific embodiment 50, wherein said proliferative disease is located on the skin, eye, blood, mouth (e.g., gums, etc.), throat, esophagus, digestive tract, or colon, of said patient.

Specific embodiment 52. The method according to any one of specific embodiments 45-51, wherein said proliferative disease is cancer.

Specific embodiment 53. The method according to any one of specific embodiments 45-52, wherein said proliferative disease is melanoma, leukemia, lymphoma, or retinal blastoma.

Specific embodiment 54. The method according to any one of specific embodiments 46-53, wherein the time period between said administration and said irradiation is a length sufficient to induce binding between said compound and the cells of said proliferative disease on said patient.

Specific embodiment 55. The method according to specific embodiment 54, wherein said patient is not exposed to radiation capable of separation of said photolabile group and said ubiquitin ligase binding moiety during administration and/or during said time period.

Specific embodiment 56. The method according to specific embodiment 54 or 55, wherein the time period between administration and irradiation is more than 5 minutes (e.g., more than 10 minutes, more than 20 minutes, more than 30 minutes, more than an hour, more than 6 hours, more than 12 hours, more than a day, etc.).

Specific embodiment 57. The method according to any one of specific embodiments 46-56, wherein said irradiation time period is more than 60 seconds (e.g., more than 120 seconds, more than 180 seconds, etc.).

Specific embodiment 58. The method according to any one of specific embodiments 50-57, wherein at least one portion of said specific area is irradiated for more than 30 seconds (e.g., more than 60 seconds, more than 120 seconds, more than 180 seconds, etc.).

Specific embodiment 59. The method according to any one of specific embodiments 50-57, wherein two or more portions are irradiated sequentially.

Specific embodiment 60. The method according to specific embodiment 59, wherein each of said two or more portions are irradiated for an independently selected irradiation time period based on the characteristics of said portion (e.g., proliferative disease density, type, etc.).

Specific embodiment 61. The method according to any one of specific embodiments 46-60, wherein said electromagnetic radiation has a spot size on said patient of from 0.1 mm² to 100 cm² (e.g., from 0.1 mm² to 1000 mm², from 1000 mm² to 0.1 cm², from 0.1 cm² to 10 cm² from 10 cm² to 100 cm², etc.).

Specific embodiment 62. The method according to any one of specific embodiments 46-60, wherein said electromagnetic light is monochromatic radiation having a spectral bandwidth of less than 10 nm (e.g., less than 5 nm, less than 1 nm, etc.).

Specific embodiment 63. The method according to any one of specific embodiments 45 or 51-53, wherein the separation of said photolabile group from the ubiquitin ligase binding moiety of said compound occurs following exposure to environmental light (e.g., sunlight, etc.).

Specific embodiment 64. The method according to any one of specific embodiments 45-63, wherein said compound is administered to the skin of said subject.

As various changes can be made in the above-described subject matter without departing from the scope and spirit of the present invention, it is intended that all subject matter contained in the above description, or defined in the appended claims, be interpreted as descriptive and illustrative of the present invention. Many modifications and variations of the present invention are possible in light of the above teachings. Accordingly, the present description is intended to embrace all such alternatives, modifications and variances which fall within the scope of the appended claims.

1. A compound having the structure of formula (I):
PB-L-ULB—PLG (I)
wherein ULB is a ubiquitin ligase binding moiety;
L is a linker;
PB is a protein binding moiety; and
PLG is a nitrophenyl based photolabile group;
wherein PLG is covalently bonded to ULB through a carbamate linkage;
or pharmaceutically acceptable salts thereof.

2. The compound according to claim 1, wherein the nitrogen of said carbamate linkage is a hydrogen binding moiety in ULB when said photolabile group is not present.

3. The compound according to claim 1, wherein PLG has the structure of formula (II): 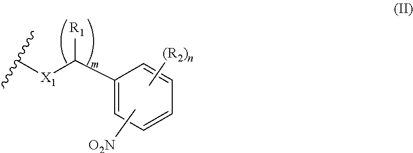
wherein 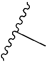
indicates the point of attachment to the ULB group;
m is 0, 1, or 2;
n is 0, 1, 2, 3, or 4;
X₁ is —O—, —C(O)—, —NR^{a}—, —OC(O)—, —C(O)O—, —NR^{a}C(O)—, or —C(O)NR^{a}—;
R₁ is independently selected at each occurrence from hydrogen, alkyl, and alkoxy;
R₂ is independently selected at each occurrence from hydrogen, —OC(O)R^{e}, —C(O)OR^{e}, —(C(R^{a})(R^{a}))₀₋₄—OC(O)N(R^{a})₂, halogen, alkyl, and alkoxy, wherein two vicinal R₂ groups do not together form a ring;
R^{a} is independently selected at each occurrence from hydrogen, and alkyl; and
R^{e} is independently selected at each occurrence from hydrogen, and alkyl.

4. The compound according to claim 3, wherein said PLG group has the structure of formula (IIc):

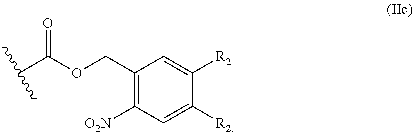

5. The compound according to claim 1, wherein said ULB binds to an E3 ubiquitin ligase.

6. The compound according to claim 5, wherein the E3 ubiquitin ligase is selected from the group consisting of von Hippel Lindau (VHL) E3 ubiquitin ligase, β-Transducin Repeat Containing (β-TRCP) E3 Ubiquitin Protein Ligase, Mouse Double Minute 2 (Mdm2) E3 Ubiquitin Protein Ligase, and a Cereblon (CRBN) E3 Ubiquitin ligase.

7. The compound according to claim 1, wherein said compound has the structure of formula 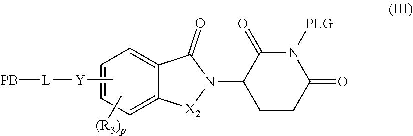
wherein p is 0, 1, 2, or, 3;
R₃ is independently selected at each occurrence from hydrogen, —N(R^{a})(R^{a}), alkyl, and alkoxy;
X₂ is C(O), CH, CR^{a}, or NR^{a};
Y is absent, —O—, —C(O)—, —NR^{a}—, —OC(O)—, —C(O)O—, —NR^{a}C(O)—, or —C(O)NR^{a}—;
R^{a} is independently selected at each occurrence from hydrogen, and alkyl.

8. The compound according to claim 1, wherein said compound has the structure of formula (IV): 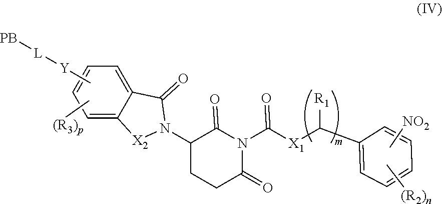
wherein m is 0, 1, or 2;
n is 0, 1, 2, 3, or 4;
p is 0, 1, 2, or 3;
R₁ is independently selected at each occurrence from hydrogen, alkyl, and alkoxy;
R₂ is independently selected at each occurrence from hydrogen, —OC(O)R^{e}, —C(O)OR^{e}, alkyl, and alkoxy, wherein two vicinal R₂ groups do not together form a ring;
R₃ is independently selected at each occurrence from hydrogen, —N(R^{a})(R^{a}), alkyl, and alkoxy;
X₁ is —O—, —C(O)—, —NR^{a}—, —OC(O)—, —C(O)O—, —NR^{a}C(O)—, or —C(O)NR^{a}—;
X₂ is C(O), CH₂, C(R^{a})(R^{a}), or NR^{a};
Y is absent, —O—, —C(O)—, —NR^{a}—, —OC(O)—, —C(O)O—, —NR^{a}C(O)—, or —C(O)NR^{a}—;
R^{a} is independently selected at each occurrence from hydrogen, and alkyl; and
R^{e} is independently selected at each occurrence from hydrogen, and alkyl.

9. The compound according to claim 1, wherein said protein binding moiety is a tyrosine kinase inhibitor, a BRAF-mutant inhibitor, or a MEK inhibitor.

10. The compound according to claim 1, wherein said protein binding moiety binds to one or more of Abelson Murine Leukemia (ABL) Proteins, Breakpoint Cluster Region Protein (BCR), BCR-ABL fusion proteins, Bromodomain and Extra Terminal Domain (BRD) Family proteins, anaplastic lymphoma kinase (ALK) protein, echinoderm microtubule-associated protein like (EML)-ALK fusion proteins.

11. The compound according to claim 1, wherein PB has an affinity for its target protein (K_{d}) of less than 1 mM.

12. The compound according to claim 1, wherein PB is: 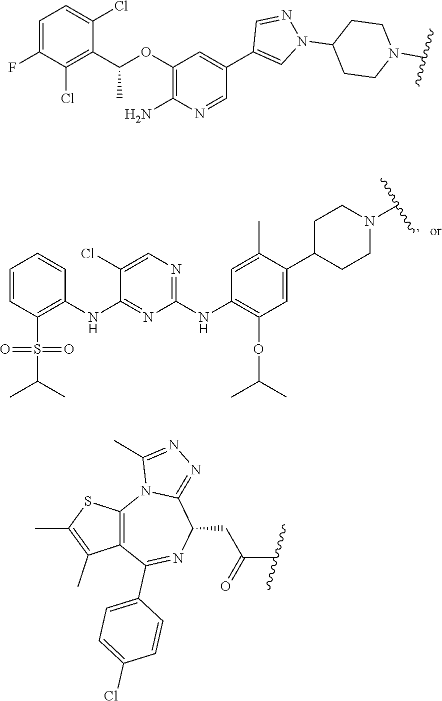 [SMILES: *N1CCC(n2cc(-c3cnc(N)c(O[C@H](C)c4c(Cl)ccc(F)c4Cl)c3)cn2)CC1 ; *N1CCC(c2cc(OC(C)C)c(Nc3ncc(Cl)c(Nc4ccccc4S(=O)(=O)C(C)C)n3)cc2C)CC1 ; *C(=O)C[C@@H]1N=C(c2ccc(Cl)cc2)c2c(sc(C)c2C)-n2c(C)nnc21]
wherein 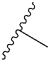
indicates the point of attachment to the L group.

13. The compound according to claim 1, wherein said compound has the structure of formula (Va) or (Vb):
PB—NH—(CH₂)₁₋₈—NH—C(O)—ULB—PLG (Va)
PB—(CH₂)₁₋₈—NH—C(O)—(CH₂)₁₋₈—ULB—PLG (Vb)

14. A compound having the structure:

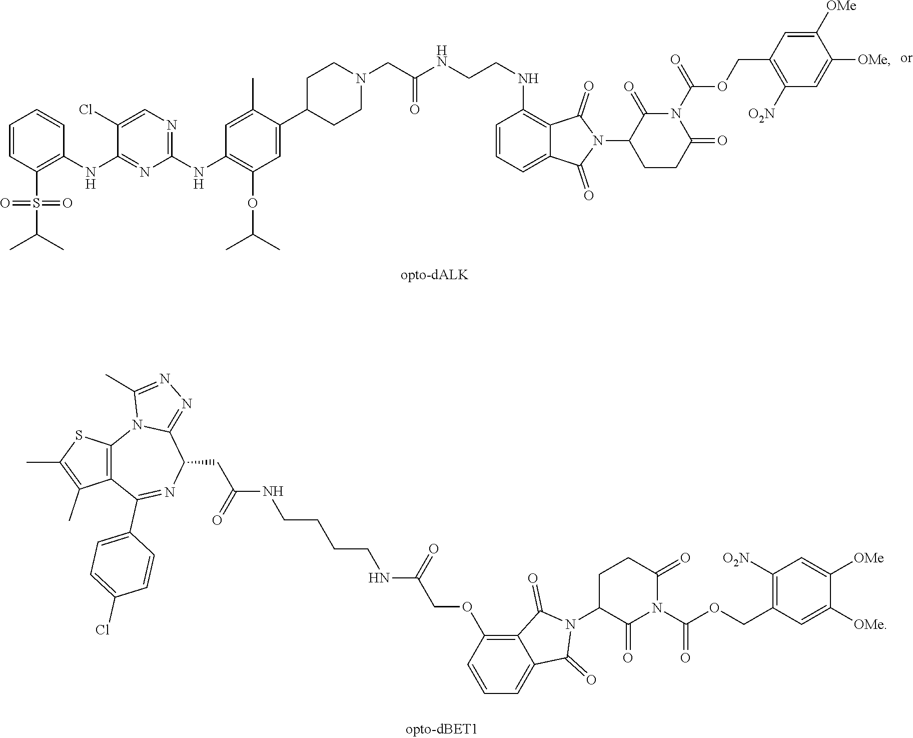 [SMILES: COc1cc(COC(=O)N2C(=O)CCC(N3C(=O)c4cccc(NCCNC(=O)CN5CCC(c6cc(OC(C)C)c(Nc7ncc(Cl)c(Nc8ccccc8S(=O)(=O)C(C)C)n7)cc6C)CC5)c4C3=O)C2=O)c([N+](=O)[O-])cc1OC ; COc1cc(COC(=O)N2C(=O)CCC(N3C(=O)c4cccc(OCC(=O)NCCCCNC(=O)C[C@@H]5N=C(c6ccc(Cl)cc6)c6c(sc(C)c6C)-n6c(C)nnc65)c4C3=O)C2=O)c([N+](=O)[O-])cc1OC]

15. A compound having the structure of formula (VI): 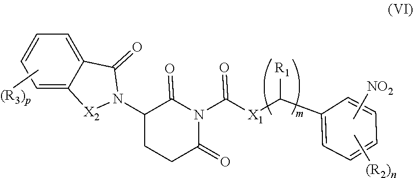
wherein m is 0, 1, or 2;
n is 0, 1, 2, 3, or 4;
p is 0, 1, 2, 3, or 4;
R₁ is independently selected at each occurrence from hydrogen, alkyl, and alkoxy;
R₂ is independently selected at each occurrence from hydrogen, —OC(O)R^{e}, —C(O)OR^{e}, alkyl, and alkoxy;
R₃ is independently selected at each occurrence from hydrogen, —N(R^{a})(R^{a}), alkyl, and alkoxy;
X₁ is —O—, —C(O)—, —NR^{a}—, —OC(O)—, —C(O)O—, —NR^{a}C(O)—, or —C(O)NR^{a}—;
X₂ is C(O), CH₂, C(R^{a})(R^{a}), or NR^{a};
R^{a} is independently selected at each occurrence from hydrogen, and alkyl; and
R^{e} is independently selected at each occurrence from hydrogen, and alkyl;
or pharmaceutically acceptable salts thereof.

16. A pharmaceutical composition comprising the compound according to claim 1 and one or more pharmaceutically acceptable salts, carriers, or diluents.

17. The pharmaceutical composition according to claim 16, wherein said composition is formulated for topical delivery.

18. A method for degrading a protein of interest, the method comprising contacting the protein of interest with a compound according to claim 1 and activating the compound with electromagnetic radiation.

19. A method for reducing the proliferation or survival of a neoplastic cell, the method comprising contacting the cell with a compound according to claim 1 and activating the compound with electromagnetic radiation.

20. A method for the treatment of a proliferative disease in a patient in need thereof comprising administering a compound according to claim 1.

21-26. (canceled)
